(19) United States
(12) Patent Application Publication
(10) Pub. No.: US 2025/0322954 A1
(43) Pub. Date: Oct. 16, 2025

(54) APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK AND/OR WITH OR USING A PERSONAL MONITORING DEVICE

(71) Applicant: RAYMOND ANTHONY JOAO, YONKERS, NY (US)

(72) Inventor: RAYMOND ANTHONY JOAO, YONKERS, NY (US)

(21) Appl. No.: 19/053,660

(22) Filed: Feb. 14, 2025

Related U.S. Application Data
(60) Provisional application No. 63/634,841, filed on Apr. 16, 2024.

(51) Int. Cl. G16H 50/20; G16H 10/60; G16H 40/67; G16H 80/00; H04M 1/72421; H04M 1/72436; H04M 1/72439

(52) U.S. Cl. CPC G16H 50/20; G16H 10/60; G16H 40/67; G16H 80/00; H04M 1/72421; H04M 1/72436; H04M 1/72439

(57) ABSTRACT

An apparatus, including a central computer, a distributed ledger/blockchain technology system, a personal device having a first input device, a first processor, and a first database, and a provider device. The first input device inputs a note or message containing healthcare information regarding a healthcare condition of the individual. The first processor determines whether or not an individual has previously experienced the healthcare condition. The personal device generates a healthcare alert and transmits the healthcare alert to the central processing computer and to the provider communication device. The central processing computer updates a plurality of electronic healthcare records, electronic medical records, electronic dental records, electronic pharmacy records, or electronic behavioral healthcare records, and personal healthcare record, of the individual. The central processing computer stores information regarding the note, message, healthcare alert, and information regarding the updates in a database and in the distributed ledger and blockchain technology system.

### RELATED APPLICATIONS

This application claims the benefit of the priority of U.S. Provisional Patent Application Ser. No. 63/634,841, filed Apr. 16, 2024, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK AND/OR WITH OR USING A PERSONAL MONITORING DEVICE”, the subject matter and teachings of which are hereby incorporated by reference herein in their entirety.

### FIELD OF THE INVENTION

The present invention pertains to an apparatus and a method for providing healthcare services remotely or virtually with or using an electronic healthcare record and/or a communication network and/or with or using a personal monitoring device and, in particular, to an apparatus and a method for providing healthcare services remotely or virtually with or using an electronic healthcare record and/or a communication network and/or with or using a personal monitoring device which can facilitate communications and data and/or information exchange with and/or between an individual or a patient and a healthcare provider or a healthcare professional.

### BACKGROUND OF THE INVENTION

Healthcare is an area of major concern in the United States. Each year, tens of millions of individuals seek or need the assistance of healthcare professionals. In order to perform proper diagnoses and to prescribe appropriate treatments, healthcare professional or providers typically rely on information which is obtained from patients, relatives of patients, previous providers, and/or healthcare facility and/or hospital staff members. The need to have accurate and/or up-to-date data and/or information, in providing healthcare services and/or healthcare-related services, cannot be emphasized enough.

Stories constantly emerge about patients receiving the wrong treatments, having the wrong surgical procedures performed on themselves, receiving a drug or drugs which fatally and/or otherwise adversely interact with another drug or drugs, etc., with stories going on and on. A 1999 study estimated that between 44,000 and 98,000 individuals die annually in hospitals, in the United States alone, as the result of errors or mistakes made by doctors, healthcare providers, and/or healthcare facility workers. Various other reports also estimate 225,000 deaths annually from medical errors and 180,000 deaths annually from medication errors and adverse reactions. There is no doubt that many of these deaths result from inaccurate and/or erroneous information and/or the lack of the availability of correct and/or up-to-date information.

Another problem lies with the fact that the main source of patient information, medical histories, family histories, etc., upon which doctors or providers may base their diagnoses and/or treatments, are patients who usually supply this information on questionnaires or forms just prior to seeing the healthcare provider and/or during a preliminary interview with the provider. In this regard, information obtained from these questionnaires or forms, as well as from these preliminary interviews with the providers, may not necessarily result in sufficient, comprehensive, and/or accurate, information being obtained regarding the patient. Further, there is no guarantee that the same information will be provided, in a uniform manner, to a next or different provider. As a result, patient information may not be uniformly distributed and/or be available to providers at the point of treatment and/or otherwise.

Another problem which exists in the current healthcare system is that doctors or other providers do not always have the latest information and/or research material available to them prior to, and/or during, the diagnosis and/or treatment process.

Still other problems arise when an individual or a patient, or a caregiver for an individual or a patient, engages in a video call with a healthcare provider in or during a remote or a virtual provider visit or in or during a remote or a distance examination. In such instances, there is no guarantee that the healthcare provider has access to the individual's or the patient's healthcare records, files, or history, before or during the video call and the remote or virtual provider visit or in or during the remote or distance examination. Further, there is no guarantee that the healthcare provider, even if provided with access to the individual's or the patient's healthcare records, files, or history, can access the same, and enter notes, findings, and/or information, into the same in an easy and efficient manner, and/or be able to do so in, during, or after the remote or distance examination. As a result, the healthcare provider may be not be provided with sufficient information to conduct a full and complete examination or evaluation of the individual or the patient, or to make or arrive at a diagnosis based on any and/or all needed and available information regarding the individual or the patient, or to prescribe a treatment based on any and/or all needed or available information regarding the individual or the patient. As a further result, the healthcare provider may not have access to the individual's or the patient's healthcare records, files, or history, in order to properly or completely enter notes or any other information regarding the individual or the patient as well as any information, observations, examination findings, or examinations results, obtained by, made by, or obtained during the remote or the virtual provider visit or in or during the remote or the distance examination.

Another problem associated with healthcare providers and patients who engage in video calls during remote or virtual provider visits or in or during remote or distance examinations lies in the fact that the healthcare provider may not able to control and/or to monitor healthcare equipment, devices, or systems, which is located with the patient. In such instances, patient control and/or operation of such healthcare equipment, devices, or systems, might fail to yield measurements or results if uncalibrated and/or misused by patients or their caregivers.

Another problem also lies in securing data and/or information stored in patient's healthcare records, files, or history, especially when data and/or information may be accessed via the online networks and/or the Internet and/or the World Wide Web.

Various and respective electronic healthcare records products and/or electronic healthcare records systems exist commercially in the marketplace. There are also numerous electronic healthcare records products and/or electronic healthcare records systems suppliers or providers in the marketplace, and each of these respective suppliers or providers offer one or more of their own and/or proprietary electronic healthcare records products and/or electronic healthcare records systems.

Healthcare providers and healthcare provider groups can typically use one or more of these electronic healthcare records products and/or electronic healthcare records systems in their practices. Typically, these various and respective electronic healthcare records products and/or electronic healthcare records systems, which are offered by these various, different, and/or respective, suppliers or providers cannot interoperate with, and are not interoperable with, those electronic healthcare records products and/or electronic healthcare records systems which are offered by other suppliers or providers.

As a result, no individual or patient healthcare data and/or information can be shared with and/or between the various electronic healthcare records which are used by the various healthcare providers or healthcare provider groups of or for an individual or patient. It is submitted that this lack of interoperability can result in incomplete healthcare recordkeeping, inefficient care for the individual or patient, duplicate or redundant testing and procedures, and duplicate or redundant charges and payments for the same, incomplete or incorrect diagnoses and treatments, and such can potentially result in unnecessary, incorrect, and possibly fraudulent, charges and/or billing.

It is submitted that, while electronic healthcare records products and/or electronic healthcare records systems, provided by one supplier or provider, can aid a healthcare provider or healthcare provider group by making healthcare recordkeeping for an individual or patient somewhat easier and more efficient, and/or easier and more efficient within a practice, the lack of the healthcare data and/or information for the individual or patient, and/or the lack of access to the same, which is stored in electronic healthcare records products and electronic healthcare records systems provided by other or different healthcare records products and/or electronic healthcare records systems suppliers or providers, and/or which are used by other healthcare providers of or for the individual or patient, renders an individual's or patient's electronic healthcare records incomplete.

It is submitted that the inability of electronic healthcare records products and/or electronic healthcare records systems, which are offered by various suppliers or providers, to interoperate with, and/or to be interoperable with, each other and/or with all of the other electronic healthcare records products and/or electronic healthcare records systems used by all of the various healthcare providers of or for an individual or patient, can result in the afore-mentioned incomplete healthcare recordkeeping, inefficient care for the individual or patient, duplicate or redundant testing and procedures, and charges and payments for the same, incomplete or incorrect diagnoses and treatments, and such can potentially result in unnecessary, incorrect, and possibly fraudulent, charges and billing.

In view of the foregoing, it is submitted that there is no effective and efficient electronic healthcare records product or electronic records system which can effectively and efficiently interoperate with, and/or be interoperable with, those respective electronic healthcare records products or electronic records systems which are offered by various other and/or different suppliers and/or providers of electronic healthcare records products and/or electronic healthcare records systems. It is further submitted that there is also no effective and efficient electronic healthcare records product or electronic records system which can be used by an individual or patient in order to enter information into his or her own electronic healthcare record(s) which can be interoperable with, and share this information, with those electronic healthcare records products or electronic records systems which are used by the individual's or patient's various healthcare providers.

In view of the foregoing, it is submitted that there are numerous shortcomings in the prior art telehealth technology and prior art electronic healthcare records products and systems technology which need to be addressed and solved with and by technical solutions and improvements to and over existing technology.

### SUMMARY OF THE INVENTION

The present invention pertains to an apparatus and a method for providing healthcare services remotely or virtually with or using an electronic healthcare record and/or a communication network and/or with or using a personal monitoring device and, in particular, to an apparatus and a method for providing healthcare services remotely or virtually with or using an electronic healthcare record and/or a communication network and/or with or using a personal monitoring device which can facilitate communications and data and/or information exchange with and/or between an individual or a patient and a healthcare provider or a healthcare professional, which overcomes the shortcomings of the prior art.

The present invention can be utilized to provide healthcare services, such as, but not limited to, a consultation with a healthcare provider, a discussion with a healthcare provider, a remote or virtual office visit with a healthcare provider, a healthcare examination by a healthcare provider, remotely or virtually via a video telephone call, a video chat session, or a videoconference, with and between an individual, a patient, or a caregiver for or an individual or a patient, and a healthcare provider.

The present invention can be utilized to all an individual or a patient to self-monitor and/or to self-record and/or automatically record information regarding his or her health, health condition, wellness, wellness condition, fitness, and/or fitness condition and/to self-report and/or automatically report the respective condition or conditions to a healthcare provider, a healthcare insurer or healthcare payer, a provider of healthcare or healthcare-related products and/or services, a provider of wellness or wellness-related products and/or services, or a provider of fitness or fitness-related products or services, with or using a user communication device as a personal monitoring device.

The present invention can also be utilized to provide various services, such as, but not limited to, a consultation with a wellness provider or fitness provider, a discussion with a wellness provider or fitness provider, a remote or virtual office visit with a wellness provider or fitness provider, a healthcare examination by a wellness provider or fitness provider, remotely or virtually via a video telephone call, a video chat session, or a videoconference, with and between an individual, a patient, or a caregiver for or an individual or a patient, and a wellness provider or fitness provider.

The present invention can also be utilized to provide healthcare services, such as, but not limited to a consultation with a healthcare insurer, a healthcare payer, or an intermediary or a discussion with a healthcare insurer, a healthcare payer, or an intermediary, remotely or virtually via a video telephone call, a video chat session, or a videoconference, with and between an individual, a patient, or a caregiver for or an individual or a patient, and a healthcare insurer or a healthcare payer or an intermediary.

The present invention can be utilized to facilitate remote or virtual provider visits, consultations, and/or examinations, via and/or through the use of video calls, video chat sessions, and/or videoconferences, with and between an individual, a patient, and/or a caregiver for the individual or the patient, and a healthcare provider and/or with a wellness provider or a fitness provider. The apparatus and method of the present invention can also be utilized to facilitate remote or virtual conferences, discussions, and/or consultations, via and/or through the use of video calls, video chat sessions, and/or videoconferences, with and between an individual, a patient, and/or a caregiver for the individual or the patient, a healthcare provider, a wellness provider, a fitness provider, a healthcare insurer or a healthcare payer, and/or an intermediary.

The present invention can also be utilized to provide healthcare services, such as, but not limited to a consultation with a healthcare provider, a discussion with a healthcare provider, a remote or virtual office visit with a healthcare provider, a healthcare examination by a healthcare provider, remotely or virtually via a video telephone call, a video chat session, or a videoconference, with and between an individual, a patient, or a caregiver for or an individual or a patient, and a healthcare provider. The present invention can also be utilized to provide healthcare services, such as, but not limited to a consultation with a healthcare insurer, a healthcare payer, or an intermediary or a discussion with a healthcare insurer, a healthcare payer, or an intermediary, remotely or virtually via a video telephone call, a video chat session, or a videoconference, with and between an individual, a patient, or a caregiver for or an individual or a patient, and a healthcare insurer or a healthcare payer or an intermediary.

The present invention can also be utilized to provide a healthcare provider with access to the individual's or the patient's healthcare records, files, or history, before or during a video call, a video chat session, or a videoconference, before, in or during, and/or after, a remote or a virtual provider visit or in or during a remote or a distance examination. The present invention can also be utilized to provide a healthcare provider with access to the individual's or the patient's healthcare records, files, or history, before or during an in-office visit.

In this regard, the healthcare provider can be provided with any and/or all information regarding the individual or the patient in order to conduct a full and complete examination or evaluation of the individual or the patient, or to make or arrive at a diagnosis based on any and/or all information regarding the individual or the patient, or to prescribe a treatment based on any and/or all any and/or all information regarding the individual or the patient. As a further result, the healthcare provider can be provided with access to the individual's or the patient's healthcare records, files, or history, in order to properly or completely enter notes or any other information regarding the individual or the patient as well as any information, observations, examination findings, or examinations results, obtained by, made by, or obtained during, the remote or the virtual provider visit or in or during the remote or the distance examination, so as to assure that any and/or all interactions with or between the individual or the patient and any healthcare provider, including information obtained from or during remote or the virtual provider visits or in or during remote or the distance examinations are documented in the individual's or the patient's healthcare records, files, or history.

The present invention also provides a technologically improved electronic healthcare record or EHR, and/or a technologically improved electronic healthcare record, electronic healthcare file, electronic medical record, electronic dental record, electronic pharmacy record, and/or electronic behavioral health record, and/or personal healthcare record, which provides for all of the features and/or functionalities described herein. The present invention also provides a technologically improved electronic healthcare record or EHR, and/or a technologically improved electronic healthcare record, electronic healthcare file, electronic medical record, electronic dental record, electronic pharmacy record, and/or electronic behavioral health record, and/or personal healthcare record, which provides for all of the features and/or functionalities described herein, including, but not limited to a respective record which allows for the entry, and/or storage, and/or the retrieval, of data and/or information which can be provided by an individual or a patient, or a caregiver of or for the individual or patient, and/or which can contain text, audio information, a picture(s) or photograph(s), and/or video information, and/or any combination of the same.

The present invention includes a central processing computer and distributed ledger and Blockchain technology system (hereinafter “central processing computer/distributed ledger/Blockchain technology system” which includes a central processing computer component and a distributed ledger and Blockchain technology system component. The present invention also includes a provider communication device, or any number of provider communication devices, which is associated with a healthcare provider such as a healthcare professional, a hospital, a clinic, a pharmacy, a treatment center, a treatment facility, and/or any other provider of services described herein, as well as with any of the various medical specialists, internists, orthopedists, ophthalmologists, cardiologists, hematologists, endocrinologists, oncologists, ears, nose and throat specialists, neurologists, urologists, gastroenterologists, dermatologists, pediatricians, surgeons, surgical specialists, physicians, dentists, endodontists, periodontists, oral surgeons, orthodontists, prosthodontists, dental hygienists, or other dental or oral healthcare providers, psychiatrists, psychologists, optometrists, podiatrists, osteopaths, chiropractors, pharmacists, therapists, physical therapists, respiratory therapists, nurses, healthcare aids, nutritionists, wellness trainers, wellness consultants, or wellness coaches, fitness trainers, fitness consultants, or fitness coaches, and/or any other persons, individuals and/or professionals who can provide healthcare, healthcare-related, wellness and/or wellness-related, and/or fitness and/or fitness-related, services and/or products.

The present invention can also include a healthcare insurer or payer communication device or computer (hereinafter referred to as “insurer/payer communication device” or “insurer/payer computer”) which is associated with a healthcare payer such as a healthcare insurer, insurance company, health maintenance organization, a clinic, and/or any other payer of healthcare services and products described herein.

The present invention can also include a user, patient, or individual, communication device or computer or personal monitoring device (which may hereinafter be referred to as “user communication/personal monitoring device”) which can be used by or associated with any user of the present invention or any individual or healthcare patient or client who seeks or who is provided with healthcare and/or related services, products and/or related information. The user communication/personal monitoring device can also be utilized by any individual, party, or entity, who or which may merely utilize the present invention in order to care or another individual or to obtain information of interest. The user communication/personal monitoring device can be wired device or can be a wireless device.

The present invention can also include a governmental entity computer or intermediary communication device or computer (hereinafter referred to as “intermediary communication device” or “intermediary computer”), a healthcare records computer or computers (hereinafter also referred to as “healthcare records computer”, an insurance exchange computer, a social networking computer, and/or a media computer.

Each of the central processing computer/distributed ledger/Blockchain technology system, or the central processing computer component of the same, the provider communication device(s), the insurer/payer communication device(s), the user communication/personal monitoring device(s), the intermediary communication device(s), the healthcare records computer(s), the insurance exchange computer(s), the social networking computer(s), and/or the media computer(s), can communicate with each other in a bi-directional manner with, and/or can send and/or receive signals, messages, reports, notification messages, alerts, or any other communications or electronic communication transmissions, to, from and/or between, any other, or any number of, other central processing computer/distributed ledger/Blockchain technology systems, or the central processing computer components of the same, provider communication devices, insurer/payer communication devices, user communication/personal monitoring devices, intermediary communication devices, healthcare records computers, insurance exchange computers, social networking computers, and/or media computers.

Each of the central processing computer/distributed ledger/Blockchain technology system, or the central processing computer component of the same, the provider communication device(s), the insurer/payer communication device(s), the user communication/personal monitoring device(s), the intermediary communication device(s), the healthcare records computer(s), the insurance exchange computer(s), the social networking computer(s), and/or the media computer(s), can be linked to or with any other. Each of the central processing computer/distributed ledger/Blockchain technology system, or the central processing computer component of the same, the provider communication device(s), the insurer/payer communication device(s), the user communication/personal monitoring device(s), the intermediary communication device(s), the healthcare records computer(s), the insurance exchange computer(s), the social networking computer(s), and/or the media computer(s), via a wired link or line or via a wireless link.

Any and/or all of the signals, messages, reports, notification messages, or any other communications, described herein as being transmitted from any one respective device, computer, or communication device, to another, can be, or can be included in, or be attached to, an e-mail message, an instant messaging message, an electronic transmission, or an electronic data transmission or electronic data interchange, or can be transmitted via and/or in any other data or information transmission, and/or can be transmitted via or using any appropriate or necessary computer(s) or device(s).

The present invention is utilized on, and/or over, the Internet and/or the World Wide Web. The present invention can also utilize wireless Internet and/or World Wide Web services, equipment and/or devices. The central processing computer component of the central processing computer/distributed ledger/Blockchain technology system has a web site or web sites associated therewith. Each of the other respective computers or communication devices described herein can also have a web site or web sites associated with same.

Although the Internet and/or the World Wide Web is a preferred communication system and/or medium utilized, the present invention can also be utilized with any appropriate communication network or system including, but not limited to, a communication network or system, a telecommunication network or system, a telephone communication network or system, a cellular communication network or system, a wireless communication network or system, a line or wired communication network or system, a wireless Internet network or system, a wireless World Wide Web network or system, a digital communication network or system, a personal communication network or system, a personal communication services (PCS) network or system, a satellite communication network or system, a broad band communication network or system, a low earth orbiting (LEO) satellite network or system, a public switched telephone network or system, a telephone communication network or system, a radio communication network or system, a cable television network or system, and/or any other communication network or system, and/or any combination of the above communication networks or systems.

The present invention can also include any number of RFID tags/RFID reader system(s), each of which can include any number of RFID tags and RFID reader systems. The RFID tags/RFID reader system(s) can be utilized in or for any number of a variety ways as described herein and/or otherwise. The RFID tags/RFID reader system(s) can be connected to, and/or linked with, the central processing computer/distributed ledger/Blockchain technology system, using any suitable and/or appropriate wired connection, wireless connection, or any combination of same, on, via, or using, any suitable and/or appropriate communication network or any combination of communication networks.

Any user, individual, patient, caregiver, and/or any healthcare provider, healthcare professional, hospital, clinic, pharmacy, treatment center, treatment facility, and/or any other provider of services, healthcare or otherwise, described herein, who or which uses the present invention, can be assigned a healthcare account or any number of healthcare accounts. Any user, individual, patient, caregiver, and/or any healthcare provider, healthcare professional, hospital, clinic, pharmacy, treatment center, treatment facility, and/or any other provider of services or goods, healthcare, wellness, fitness, or otherwise, described herein can also be assigned any number of types or kinds of healthcare accounts depending upon how they respectively utilize the present invention.

Any of the herein-described insurers, payers, intermediaries, governmental entities, an/or any other persons or entities who or which utilize the present invention for his, her, or its, respective purposes can also be assigned one or more healthcare accounts.

An RFID tag can be assigned to any user, individual, patient, caregiver, and/or any healthcare provider, healthcare professional, hospital, clinic, pharmacy, treatment center, treatment facility, and/or any other provider of services or goods, described herein, healthcare, wellness, fitness, or otherwise, who or which uses the present invention. The RFID tag can also be assigned to a healthcare account which is assigned to or for the respective user, individual, patient, caregiver, healthcare provider, healthcare professional, hospital, clinic, pharmacy, treatment center, treatment facility, and/or any other provider of services or services, healthcare, wellness, fitness, or otherwise, described herein, who or which uses the present invention.

The RFID tag can be provided in or as a physical identification card or other physical item or device, and/or can be attached to, or located within, an article of clothing, a watch, a necklace, a bracelet, an ankle bracelet, a ring, an article of jewelry, or any other wearable article or item. The RFID tag can also be implantable within an individual or patient. The RFID tag 92A can also be provided in or as a physical identification card, and/or the RFID tag can be attached to, or located on or within, or situated on, a cellular telephone, a smart phone, a Smartphone, a personal digital assistant, a watch, a smartwatch, or on a watch, a smartwatch, a necklace, a bracelet, an ankle bracelet, a ring, an article of jewelry, or any other wearable article or item or any other personal article or item.

An RFID reader system, or any RFID reader or RFID readers of same, can be located at any facility or office, or in a room or venue therein, of any user, individual, patient, caregiver, and/or healthcare provider, healthcare professional, hospital, clinic, pharmacy, treatment center, treatment facility, and/or any other provider of services, healthcare, wellness, fitness, or otherwise, described herein, who or which utilizes the present invention. An RFID reader system, or any RFID reader or RFID readers of same, can also be located on or implemented with any communication device, computer, computer peripheral device, cellular telephone, smart phone, personal digital assistant, or any other device of or associated with, any user, individual, patient, caregiver, and/or healthcare provider, healthcare professional, hospital, clinic, pharmacy, treatment center, treatment facility, and/or any other provider of services, healthcare, wellness, fitness, or otherwise, described herein, who or which utilizes the present invention.

The RFID tag/RFID reader system(s), and any RFID tags and any RFID reader system(s) of same, can be utilized for identifying, verifying the identification of, and/or for accessing, any data and/or information, and/or any account(s) or any healthcare account(s) of, associated with, corresponding to, or regarding, any user, individual, patient, caregiver, and/or healthcare provider, healthcare professional, hospital, clinic, pharmacy, treatment center, treatment facility, and/or any other provider of services, healthcare, wellness, fitness, or otherwise, described herein, who or which utilizes the present invention.

The present invention can also include any number of user/patient monitoring systems which can include any number of monitoring devices and/or healthcare measurement tools or devices for detecting, recording, measuring, and/or monitoring, an individual's or patient's heart rate, pulse rate, blood pressure, blood oxygen level(s), blood sugar level(s), or any other biometric measure, measurement, or metric, as well as can include any number of performance measurement devices or tools for detecting, recording, measuring, and/or monitoring, an individual's or patient's physical performance in or during any type or kind of activity, such as, but not limited to, the individual's or patient's rate of speed, rate of acceleration, rate of deceleration, rate of changing direction of movement, distance traveled, speed of travel, and/or any other physical performance measure, measurement, or metric.

The user/patient monitoring systems can be, or can include, any wearable or non-wearable device or equipment which can be utilized to measure biological, physiological, or any other, data and/or information of, for, or related to, an individual or patient. For example, the user/patient monitoring systems can be, or can include, equipment for measuring pulse rate, heart rate, blood pressure, blood-sugar level, blood oxygen levels or percentages, blood glucose level or blood-sugar level, speed of movement, steps taken, distance traveled, or any other data and/or information regarding the individual or patient, his or her biometric data, and/or a measure of or regarding his or her performance of or in certain physical and/or non-physical tasks or activities.

The user/patient monitoring systems can be or can include, a thermometer, a digital thermometer, a stethoscope, a heart rate monitor or measurement device, a pulse rate monitor or measurement device, a blood pressure monitor or measurement device, a blood oxygen or percentage oxygen measurement device, a blood pressure measurement device, blood analysis device or machine, a respirator, a respiration monitoring or measurement device, a dialysis machine, a dialysis device, electrocardiograph (EKG) machine or device, electrocephalograph (EEG) machine or device, electromyograph (EMG) machine or device, a magnetic resonance imaging (MRI) machine or device, X-ray machine or device, medical imaging machine or device, thermal imaging machine or device, a heart sound monitor or measurement device, a lung sound monitoring or measurement device, respiration rate monitoring or measurement device, a laparascopic device, an arthroscopic device, a vascular testing device, a catheter device, a cardiac performance testing, monitoring, or measurement device, a pulmonary performance testing, monitoring, or measurement device, a vascular system performance monitoring or measurement device, a vascular system testing, monitoring, or measurement device, a metabolism monitoring or measurement device, a sonogram imaging device, a sonogram measurement device, a sonograph device, an optical response device, an optical response measurement device, an intravenous device, an arterial blood pressure measurement or monitoring device, a respiration rate measurement or monitoring device, an ultrasound imaging device, an ultrasound measurement device, a CAT SCAN device, a PET scan device, an optical metabolism measurement or monitoring device, a radiotelemetric device, a doppler medical device, a mammogram device, a carbon dioxide detection or measurement device, a carbon monoxide detection or measurement device, a transvascular impedance measurement or monitoring device, an ultrasonic imaging device, a bone conduction device, a brain function scan analyzer device, external pulse cardiac monitoring or measurement device, a fetal heart rate measurement, monitoring, or probing, device, an endotracheal cardiac monitoring device, a finger tip blood pressure monitoring device, a psychological monitoring device, a surgical instrument, a vital signs measurement device, ear pressure regulating device, phonocardiograph device, acoustic aneurysm detector device, blood oxygen detection device, esophageal probing device, ultrasonic probing device, ausculoscope, vital signs monitoring system, heart activity monitoring device, pulmonary activity monitoring device, sphygmomanometer, esophageal stethoscope, venous pressure measuring device, differential doppler device, physiological data measuring device, body tissue movement device, breathalyzer device, camera probing device, microscopic camera probing device, any scope having a camera and/or a light, a laparoscopic device, a laparoscopic camera device, a camera used in or associated with equipment for performing visual probing of the body or any portions, parts, or organs, thereof, such as, but not limited to, a colonoscopy, an endoscopy, an esophagoscopy, a gastroscopy, or any devices used in laparoscopic surgery or other surgeries, an ingestible or implantable pill, capsule, or device, containing a camera and/or a light and equipment or circuitry for transmitting information as well as images to equipment inside or outside the body, and/or any other bio-metric or physiological data measuring device(s) and/or data acquisition device.

The user/patient monitoring systems can also be or can also include, a gyroscope, an accelerometer, a decelerometer, a magnetometer, an RFID tag(s), a thermometer for measuring temperature of the environment, a device for measuring body temperature, a device for measuring speed of movement, a device for measuring distance traveled, or any other device or devices which can measure and record information regarding three-dimensional movements of the individual or patient. The user/patient monitoring systems can also be, or include, any devices or equipments which are needed, required, or desired, for measuring, monitoring, and/or tracking individual or patient activity or performance using any type or kind of optical-based camera (OBC) tracking systems, local positioning system (LPS) tracking systems, and/or global positioning system/global navigation satellite system (GPS/GNSS) tracking systems, and/or any other tracking system, which are or can be utilized to track players or athletes during sporting or athletic competitions or training.

The respective optical-based camera (OBC) tracking systems, local positioning system (LPS) tracking systems, and/or global positioning system/global navigation satellite system (GPS/GNSS) tracking systems, which are utilized to track individual or patient activity or performance can also utilize or include any of the herein-described RFID tags/RFID reader system(s).

The user/patient monitoring systems can be utilized in or for any number of a variety ways as described herein and/or otherwise. The user/patient monitoring systems can be connected to, and/or linked to or with, the central processing computer/distributed ledger/Blockchain technology system, using any suitable and/or appropriate wired connection, wireless connection, or any combination of same, on, via, or using, any suitable and/or appropriate communication network or any combination of communication networks.

The user/patient monitoring systems, or any device or equipment of the same, can also be connected to, integrated in or within, and/or linked to or with, any user communication/personal monitoring device(s), using any suitable and/or appropriate wired connection, wireless connection, or any combination of same, on, via, or using, any suitable and/or appropriate communication network or any combination of communication networks. Any of device or component, which is one of or included in the user/patient monitoring systems, can also be linked with a user communication/personal monitoring device using Bluetooth technology, WiFI technology, and/or any other wireless communication technology or wireless protocol. As and for at least one example, contacts, electrodes, sensors, or leads, of a respective EKG device, thermometer, blood pressure monitor, heart rate monitor, blood sugar monitor, a blood oxygen monitor, or other or respective software application, can be connected to a user communication/personal monitoring device, which can be a smartphone or a smartwatch, via a Bluetooth or other suitable wireless connection.

The various computers and communication devices described herein can be specially programmed to perform all of their respective functions and functionalities described as being performed by the same as such are described in each of U.S. patent application Ser. No. 16/202,448, U.S. patent application Ser. No. 14/638,934, U.S. Provisional Patent Application Ser. No. 61/971,225, U.S. patent application Ser. No. 17/224,838, U.S. patent application Ser. No. 18/233,321, U.S. patent application Ser. No. 18/225,734, U.S. patent application Ser. No. 18/223,700, U.S. Pat. Nos. 7,464,040, 7,490,048, and 10,571,284, the subject matter and teachings of all of which are herein incorporated by reference in their entirety as if to be fully restated herein.

Any and/or all of the various operations, transactions, functions, and/or functionalities, described herein as being performed by present invention can also be performed by either the central processing computer component of the central processing computer/distributed ledger/Blockchain technology system and/or by the distributed ledger and Blockchain technology system component of same, or both.

The central processing computer component of the central processing computer/distributed ledger/Blockchain technology system can include a CPU, a random access memory (RAM) device(s), a read only memory (ROM) device(s), a user input device, a display device, a transmitter(s), a receiver(s), a database, and output device, and/or any other components and/devices.

Each of the provider communication devices, the insurer/payer communication devices, and/or the user communication/personal monitoring devices, and/or each of the respective intermediary computers, healthcare records computer, insurance exchange computers, social networking computers, and/or media computers, can also include a CPU, a random access memory (RAM) device(s), a read only memory (ROM) device(s), a user input device, a display device, a transmitter(s), a receiver(s), a database, and output device, a video and/or audio recording device, a global positioning system (GPS) device, a functional equipment system or device, and/or any other components and/devices.

The present invention can be utilized in connection with human beings as well as pets and/or other non-human animals. The present invention can also be utilized for, and in connection with or in conjunction with, pets and/or other non-human animals, and can be used with, and in connection with or in conjunction with, electronic veterinary records and/or electronic veterinary records systems. The user communication/personal monitoring device can be adapted for use with, and in connection with or in conjunction with, any pet or any other non-human animal, and/or can adapted to be attached to, and/or worn by, the respective pet or other non-human animal, and/or any device or garment worn by the respective pet or other non-human animal.

Each of the central processing computer component(s), each of the provider communication devices, each of the insurer/payer communication devices, and each of the user communication/personal monitoring devices, can perform any and/or all of the interoperability functions and/or processing operations or routines for and/or involving any and/or all of the herein-described various electronic healthcare records, electronic medical records, electronic dental records, electronic pharmacy records, and/or electronic behavioral healthcare records, and/or personal healthcare records, in any appropriate manner.

Copies of any and/or all updated and/or current and various electronic healthcare records, electronic medical records, electronic dental records, electronic pharmacy records, and/or electronic behavioral healthcare records, and/or personal healthcare records, for an individual or patient can be stored at or in each of the central processing computer component(s), each of the provider communication devices, each of the insurer/payer communication devices, and each of the user communication/personal monitoring devices.

In this regard, the various electronic healthcare records, electronic medical records, electronic dental records, electronic pharmacy records, and/or electronic behavioral healthcare records, and/or personal healthcare records, can be updated on any one of the central processing computer component(s), the provider communication devices, the insurer/payer communication devices, and/or the user communication/personal monitoring devices, and the updates electronic healthcare records, electronic medical records, electronic dental records, electronic pharmacy records, and/or electronic behavioral healthcare records, and/or personal healthcare records, can be transmitted to and stored at each of the each of the central processing computer component(s), each of the provider communication devices, each of the insurer/payer communication devices, and each of the user communication/personal monitoring devices.

Copies of any and/or all updated and/or current and various electronic healthcare records, electronic medical records, electronic dental records, electronic pharmacy records, and/or electronic behavioral healthcare records, and/or personal healthcare records, for an individual or patient can be stored at or in each of the central processing computer component(s), and each of the provider communication devices, each of the insurer/payer communication devices, and each of the user communication/personal monitoring devices can request access to the same, can receive and update the same, and can transmit any and/or all of the updated electronic healthcare records, electronic medical records, electronic dental records, electronic pharmacy records, and/or electronic behavioral healthcare records, and/or personal healthcare records, back to the central processing computer component(s) for storage in the database of the same.

Copies of any and/or all updated and/or current and various electronic healthcare records, electronic medical records, electronic dental records, electronic pharmacy records, and/or electronic behavioral healthcare records, and/or personal healthcare records, for an individual or patient can be stored at or in each of the central processing computer component(s), and each of the provider communication devices, each of the insurer/payer communication devices, and each of the user communication/personal monitoring devices can request access to the same, and can update the same via the central processing computer.

Any and/or all of the herein-described interoperability functions and/or processing operations or routines can be performed by the respective central processing computer component(s), provider communication devices, insurer/payer communication devices, and user communication/personal monitoring devices, automatically and/or under user control. Any of the central processing computer component(s), the provider communication devices, the insurer/payer communication devices, and/or the user communication/personal monitoring devices, can perform any of the herein-described interoperability functions and/or processing operations or routines with or using artificial intelligence (AI) software programs, software algorithms, software applications, machine language software programs, software algorithms, software applications, and/or natural language software programs, software algorithms, software applications.

The present invention can provide for the interoperability and/or data and/or information sharing between any of the herein described electronic healthcare records, electronic medical records, electronic dental records, electronic pharmacy records, and/or electronic behavioral healthcare records, and/or personal healthcare records.

The present invention can provide for the interoperability and/or data and/or information sharing between any of the herein described electronic healthcare records, electronic medical records, electronic dental records, electronic pharmacy records, and/or electronic behavioral healthcare records, and/or personal healthcare records, by using look-up tables, master index tables, data and/or information conversion information, and/or any other data and/or information, and/or by using software programs, software algorithms, software applications, which are needed or required for facilitating and for providing records interoperability operations, functions, and/or processing routines.

The present invention can also utilize artificial intelligence (AI) software programs, software algorithms, software applications, machine language software programs, software algorithms, software applications, and/or natural language software programs, software algorithms, software applications, in performing any and/or all of the functions, functionalities, processing routines, and/or operations, described herein as being performed by the present invention and/or its various component computers or communication devices.

The present invention can be utilized in order to allow an individual or patient to enter information into, and/or to change, alter, or modify, data and/or information in, his or her personal healthcare record. In this embodiment, any and/or all of the individual's or patient's various healthcare records, electronic healthcare records, electronic medical records, electronic dental records, electronic pharmacy records, and/or electronic behavioral healthcare records, and/or personal healthcare records, can be updated with any and/or all of the herein-described data, information, messages, and/or reports.

The present invention can also be utilized in order to allow an individual or patient to self report a healthcare condition or a healthcare emergency via the user communication/personal monitoring device, and/or to allow the user communication/personal monitoring device to automatically report a healthcare condition or a healthcare emergency regarding the individual or patient.

The present invention can provide, and/or provides, for the interoperability of the various electronic healthcare records, electronic medical records, electronic dental records, electronic pharmacy records, and/or electronic behavioral healthcare records, and/or personal healthcare records, of and for individuals or patients, with each other, thereby facilitating the storage and maintaining of complete and up-to-date electronic healthcare records, electronic medical records, electronic dental records, electronic pharmacy records, and/or electronic behavioral healthcare records, and/or personal healthcare records, for individuals or patients.

The present invention can provide, and/or provides, electronic healthcare records, electronic medical records, electronic dental records, electronic pharmacy records, and/or electronic behavioral healthcare records, and/or personal healthcare records, of and for individuals or patients, which are interoperable with other electronic healthcare records, electronic medical records, electronic dental records, electronic pharmacy records, and/or electronic behavioral healthcare records, and/or personal healthcare records, which are offered by various and different suppliers or providers of the same.

The present invention can provide, and/or provides, a personal healthcare record which facilitates data and/or information entry by an individual or patient and provides for the sharing of such entered data and/or information with all of the electronic healthcare records, electronic medical records, electronic dental records, electronic pharmacy records, and/or electronic behavioral healthcare records, of or used by each of, or all of, the various healthcare providers and/or healthcare insurers or payers of or for the individual or patient.

The present invention, the methods for utilizing the same, and the various components of the apparatus, including, but not limited to, the computers and/or communications devices described herein, serve to provide significant technological solutions in and to, and represent and evidence significant and unconventional improvements to technology in, the fields of telehealth platforms and electronic healthcare records products and/or systems.

### BRIEF DESCRIPTION OF THE DRAWINGS

In the Drawings:

FIG. 1 illustrates a preferred embodiment of the apparatus of the present invention, in block diagram form;

FIG. 2 illustrates the central processing computer component of the apparatus of FIG. 1, in block diagram form;

FIG. 3 illustrates the provider communication device of the apparatus of FIG. 1, in block diagram form;

FIG. 4 illustrates the insurer or payer communication device of the apparatus of FIG. 1, in block diagram form;

FIG. 5 illustrates the user communication/personal monitoring device of the apparatus of FIG. 1;

FIG. 6 illustrates another preferred embodiment of the personal monitoring device of the apparatus of FIG. 5 in a 3-D perspective;

FIG. 7 illustrates an inverted view of the personal monitoring device of FIG. 6 illustrating the underside of the same in a 3-D perspective;

FIGS. 8A and 8B illustrate a preferred embodiment method of utilizing the apparatus of the present invention, in flow diagram form;

FIGS. 9A and 9B illustrate another preferred embodiment of the apparatus of the present invention, in block diagram form; and

FIGS. 10A and 10B illustrate another preferred embodiment of the apparatus of the present invention, in block diagram form.

### DESCRIPTION OF THE PREFERRED EMBODIMENTS

The present invention pertains to an apparatus and a method for providing healthcare services remotely or virtually with or using an electronic healthcare record and/or a communication network and/or with or using a personal monitoring device and, in particular, to an apparatus and a method for providing healthcare services remotely or virtually with or using an electronic healthcare record and/or a communication network and/or with or using a personal monitoring device which can facilitate communications and data and/or information exchange with and/or between an individual or a patient and a healthcare provider or a healthcare professional.

The present invention can be utilized to provide healthcare services, such as, but not limited to, a consultation with a healthcare provider, a discussion with a healthcare provider, a remote or virtual office visit with a healthcare provider, a healthcare examination by a healthcare provider, remotely or virtually via a video telephone call, a video chat session, or a videoconference, with and between an individual, a patient, or a caregiver for or an individual or a patient, and a healthcare provider.

The present invention can be utilized to all an individual or a patient to self-monitor and/or to self-record and/or automatically record information regarding his or her health, health condition, wellness, wellness condition, fitness, and/or fitness condition and/to self-report and/or automatically report the respective condition or conditions to a healthcare provider, a healthcare insurer or healthcare payer, a provider of healthcare or healthcare-related products and/or services, a provider of wellness or wellness-related products and/or services, or a provider of fitness or fitness-related products or services, with or using a user communication device as a personal monitoring device.

The present invention can also be utilized to provide various services, such as, but not limited to, a consultation with a wellness provider or fitness provider, a discussion with a wellness provider or fitness provider, a remote or virtual office visit with a wellness provider or fitness provider, a healthcare examination by a wellness provider or fitness provider, remotely or virtually via a video telephone call, a video chat session, or a videoconference, with and between an individual, a patient, or a caregiver for or an individual or a patient, and a wellness provider or fitness provider.

The present invention can also be utilized to provide healthcare services, such as, but not limited to a consultation with a healthcare insurer, a healthcare payer, or an intermediary or a discussion with a healthcare insurer, a healthcare payer, or an intermediary, remotely or virtually via a video telephone call, a video chat session, or a videoconference, with and between an individual, a patient, or a caregiver for or an individual or a patient, and a healthcare insurer or a healthcare payer or an intermediary.

The apparatus and method of the present invention can be utilized to facilitate remote or virtual provider visits, consultations, and/or examinations, via and/or through the use of video calls, video chat sessions, and/or videoconferences, with and between an individual, a patient, and/or a caregiver for the individual or the patient, and a healthcare provider and/or with a wellness provider or a fitness provider. The apparatus and method of the present invention can also be utilized to facilitate remote or virtual conferences, discussions, and/or consultations, via and/or through the use of video calls, video chat sessions, and/or videoconferences, with and between an individual, a patient, and/or a caregiver for the individual or the patient, a healthcare provider, a wellness provider, a fitness provider, a healthcare insurer or a healthcare payer, and/or an intermediary.

The present invention can be utilized to provide healthcare services, such as, but not limited to a consultation with a healthcare provider, a discussion with a healthcare provider, a remote or virtual office visit with a healthcare provider, a healthcare examination by a healthcare provider, remotely or virtually via a video telephone call, a video chat session, or a videoconference, with and between an individual, a patient, or a caregiver for or an individual or a patient, and a healthcare provider. The present invention can also be utilized to provide healthcare services, such as, but not limited to a consultation with a healthcare insurer, a healthcare payer, or an intermediary or a discussion with a healthcare insurer, a healthcare payer, or an intermediary, remotely or virtually via a video telephone call, a video chat session, or a videoconference, with and between an individual, a patient, or a caregiver for or an individual or a patient, and a healthcare insurer or a healthcare payer or an intermediary.

The present invention can also be utilized to provide a healthcare provider with access to the individual's or the patient's healthcare records, files, or history, before or during a video call, a video chat session, or a videoconference, before, in or during, and/or after, a remote or a virtual provider visit or in or during a remote or a distance examination. The present invention can also be utilized to provide a healthcare provider with access to the individual's or the patient's healthcare records, files, or history, before or during an in-office visit.

In this regard, the healthcare provider can be provided with any and/or all information regarding the individual or the patient in order to conduct a full and complete examination or evaluation of the individual or the patient, or to make or arrive at a diagnosis based on any and/or all information regarding the individual or the patient, or to prescribe a treatment based on any and/or all any and/or all information regarding the individual or the patient. As a further result, the healthcare provider can be provided with access to the individual's or the patient's healthcare records, files, or history, in order to properly or completely enter notes or any other information regarding the individual or the patient as well as any information, observations, examination findings, or examinations results, obtained by, made by, or obtained during, the remote or the virtual provider visit or in or during the remote or the distance examination, so as to assure that any and/or all interactions with or between the individual or the patient and any healthcare provider, including information obtained from or during remote or the virtual provider visits or in or during remote or the distance examinations are documented in the individual's or the patient's healthcare records, files, or history.

As used herein, the term “electronic healthcare record” or “EHR” means any electronic healthcare record, electronic healthcare file, electronic medical record, electronic dental record, electronic pharmacy record, and/or electronic behavioral health record, as well as any personal healthcare record, and/or any other record or record keeping system, software, hardware, or device, or any combination of same or group of same, which can contain healthcare information or healthcare-related information.

As used herein, the term “personal healthcare record” also means any information, data, record(s), and/or files, which are assigned to any individual, patient, client, or user, which can be stored in their respective personal monitoring device and/or which can be accessed with or using their respective personal monitoring device. In this manner, it is envisioned that each individual, patient, client, or user, can be provided with his or her own personal healthcare record which they can own, control, keep in their possession, and/or into which they can enter data and/or information and/or store data and/or information, and from which they can transmit or share data and/or information with any healthcare provider, healthcare insurer or healthcare payer, wellness provider, or fitness provider. It is envisioned that the individual, patient, client, or user, can grant permission to access his or her personal healthcare record and/or any data and/or information contained therein to any healthcare provider, healthcare insurer or healthcare payer, wellness provider, or fitness provider.

As used herein, the terms “individual”, “patient”, “client”, “user” or the like, or their plural forms, refers to any person, individual, patient, entity, and/or client, who or which uses the present invention, and/or who seeks and/or who receives healthcare services, healthcare-related services, healthcare-related information, wellness services, wellness-related services, fitness services, fitness-related services, and/or any of the other services and/or products provided by the present invention. The terms “individual”, “patient”, “client”, “user” or the like, or their plural forms, also refer to any athlete or participant of or in any sport or sports or any activity or activities, an athlete or a sports participant of any age, a professional athlete, a minor league athlete, an Olympic athlete, a competitive athlete, a government team athlete, a world class competition athlete, an amateur athlete, a college athlete, a high school age athlete, a child athlete, a secondary school athlete, a recreational organization athlete or member, a child recreational organization athlete or member, an adult recreational organization athlete or member, a little league athlete or member, a boys club athlete or member, a girls club athlete or member, a hobbyist athlete, or any other individual, male or female, who participate in athletic endeavors, sporting events, exercise activities, martial arts, mixed martial arts, boxing, wrestling, or physical activities, or any team sport or activity or individual sport or activity.

As used herein, the term “caregiver” or the plural of same, refers to any parent, child, agent, attorney, representative, guardian, legal guardian, or any other person or entity who or which is responsible for caring for, looking after, or otherwise taking care of any healthcare, wellness, or fitness, needs of any individual or patient. The term “caregiver” also refers to any organization, governmental entity, business, team, league, club, network, or any other entity, or any employee or agent of same, which or who oversees, manages, uses, employs, provides oversight over, owns or otherwise has playing or managing rights over, provides control over, or provides or is responsible for the healthcare of, wellness of, and/or fitness of, any of the herein-described or other athletes or sports participants.

As used herein, the terms “doctor”, “healthcare provider”, “provider”, “therapist”, “healthcare information specialist”, etc., or their plural forms, refers to any medical doctor, including any and/or all of the various medical specialists and/or specialties, including, but not limited to internists, orthopedists, ophthalmologists, cardiologists, hematologists, endocrinologists, oncologists, ears, nose and throat specialists, neurologists, urologists, gastroenterologists, dermatologists, pediatricians, medical specialists, surgeons, surgical specialists, including any and/or forms and/or types of surgeons, physicians, dentists, endodontists, periodontists, oral surgeons, orthodontists, prosthodontists, dental hygienists, or other dental or oral healthcare providers, psychiatrists, psychologists, optometrists, podiatrists, osteopaths, chiropractors, pharmacists, therapists, physical therapists, respiratory therapists, nurses, healthcare aids, nutritionists, wellness trainers, wellness consultants, or wellness coaches, fitness trainers, fitness consultants, or fitness coaches, and/or any other persons, individuals, and/or professionals who can provide healthcare, healthcare-related, wellness and/or wellness-related, and/or fitness and/or fitness-related, services and/or products.

As used herein, the terms “insurer”, “payer”, “insurance provider”, “heath insurance provider”, “life insurance provider”, “disability insurance provider”, etc., or their plural forms, refers to any insurance companies, healthcare insurance companies, disability insurance companies, property or casualty insurance companies, health maintenance organizations, healthcare providers, and any other payer and/or provider of healthcare services and/or products, who which provide and/or pay for healthcare and/or healthcare-related benefits, services, and/or products, and/or who or which provide respective health insurance, life insurance and/or disability insurance benefits, services and/or products. The terms “insurer” or “payer” can also refer to any government or governmental agency, department, of entity, which provides healthcare services or which provides payment(s) for healthcare and/or healthcare services.

As used herein, the terms “broker”, “agent”, “billing service”, “collection agent”, “manager”, intermediary”, “assistant”, etc., or their plural forms, refer to any broker, insurance broker, agent, insurance agent, intermediary, third party, billing service provider, collection agent, claim processing agent, and/or any other person, individual, and/or entity, which acts on behalf of, or for, any of the individuals, patients, doctors, healthcare providers, insurers, payers, etc., described herein.

The present invention also provides a technologically improved electronic healthcare record or EHR, and/or a technologically improved electronic healthcare record, electronic healthcare file, electronic medical record, electronic dental record, electronic pharmacy record, and/or electronic behavioral health record, and/or personal healthcare record, which provides for all of the features and/or functionalities described herein. The present invention also provides a technologically improved electronic healthcare record or EHR, and/or a technologically improved electronic healthcare record, electronic healthcare file, electronic medical record, electronic dental record, electronic pharmacy record, and/or electronic behavioral health record, and/or personal healthcare record, which provides for all of the features and/or functionalities described herein, including, but not limited to a respective record which allows for the entry, and/or storage, and/or the retrieval, of data and/or information which can be provided by an individual or a patient, or a caregiver of or for the individual or patient, and/or which can contain text, audio information, a picture(s) or photograph(s), and/or video information, and/or any combination of the same.

Applicant hereby incorporates by reference herein the subject matter and teachings of U.S. Provisional Patent Application Ser. No. 63/634,841, filed Apr. 16, 2024, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK AND/OR WITH OR USING A PERSONAL MONITORING DEVICE”, the subject matter and teachings of which are hereby incorporated by reference herein in their entirety.

Applicant hereby incorporates by reference herein the subject matter and teachings of U.S. patent application Ser. No. 16/202,448, filed Nov. 28, 2018, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK”, the subject matter and teachings of which are hereby incorporated by reference herein in their entirety, and/or as if to be fully restated herein.

Applicant hereby incorporates by reference herein the subject matter and teachings of U.S. patent application Ser. No. 14/638,934, filed Mar. 4, 2015, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK”, the subject matter and teachings of which are hereby incorporated by reference herein in their entirety, and/or as if to be fully restated herein.

Applicant hereby incorporates by reference herein the subject matter and teachings of U.S. Provisional Patent Application Ser. No. 61/971,225, filed Mar. 27, 2014, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK”, the subject matter and teachings of which are hereby incorporated by reference herein in their entirety, and/or as if to be fully restated herein.

Applicant hereby incorporates by reference herein the subject matter and teachings of U.S. patent application Ser. No. 17/224,838, filed Apr. 7, 2021, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK”, the subject matter and teachings of which are hereby incorporated by reference herein in their entirety, and/or as if to be fully restated herein.

Applicant hereby incorporates by reference herein the subject matter and teachings of U.S. patent application Ser. No. 18/233,321, filed Aug. 13, 2023, and entitled “PERSONAL MONITORING APPARATUS AND METHODS”, the subject matter and teachings of which are hereby incorporated by reference herein in their entirety, and/or as if to be fully restated herein.

Applicant hereby incorporates by reference herein the subject matter and teachings of U.S. patent application Ser. No. 18/225,734, filed Jul. 25, 2023, and entitled “PERSONAL MONITORING APPARATUS AND METHODS”, the subject matter and teachings of which are hereby incorporated by reference herein in their entirety, and/or as if to be fully restated herein.

Applicant hereby incorporates by reference herein the subject matter and teachings of U.S. patent application Ser. No. 18/223,700, filed Jul. 19, 2023, and entitled “PERSONAL MONITORING APPARATUS AND METHOD”, the subject matter and teachings of which are hereby incorporated by reference herein in their entirety, and/or as if to be fully restated herein.

Applicant hereby incorporates by reference herein the subject matter and teachings of U.S. Pat. No. 7,464,040, entitled “APPARATUS AND METHOD FOR PROCESSING AND/OR FOR PROVIDING HEALTHCARE INFORMATION AND/OR HEALTHCARE-RELATED INFORMATION”, the subject matter and teachings of which are hereby incorporated by reference herein in their entirety, and/or as if to be fully restated herein.

Applicant also hereby incorporates by reference herein the subject matter and teachings of U.S. Pat. No. 7,464,040 for its teachings regarding the data and/or information which can be contained and/or stored in the electronic healthcare records or filed disclosed therein.

Applicant hereby incorporates by reference herein the subject matter and teachings of U.S. Pat. No. 7,490,048, entitled “APPARATUS AND METHOD FOR PROCESSING AND/OR FOR PROVIDING HEALTHCARE INFORMATION AND/OR HEALTHCARE-RELATED INFORMATION”, the subject matter and teachings of which are hereby incorporated by reference herein in their entirety, and/or as if to be fully restated herein.

Applicant also hereby incorporates by reference herein the subject matter and teachings of U.S. Pat. No. 7,490,048 for its teachings regarding the data and/or information which can be contained and/or stored in the electronic healthcare records or filed disclosed therein.

Applicant also hereby incorporates by reference herein the subject matter and teachings of U.S. Pat. No. 10,571,284, entitled “PERSONAL MONITORING APPARATUS AND METHOD”, the subject matter and teachings of which are hereby incorporated by reference herein in their entirety, and/or as if to be fully restated herein.

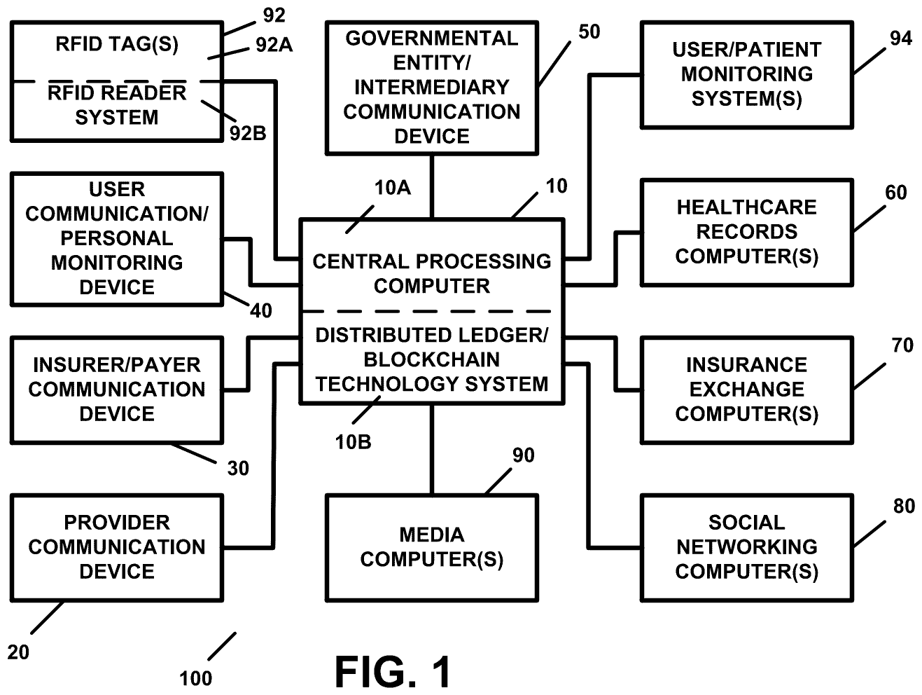

FIG. 1 illustrates the apparatus of the present invention, in block diagram form. The apparatus of the present invention is denoted generally by the reference numeral 100.

With reference to FIG. 1, the apparatus 100 includes a central processing computer and distributed ledger and Blockchain technology system 10 (hereinafter “central processing computer/distributed ledger/Blockchain technology system 10”). In a preferred embodiment, the central processing computer/distributed ledger/Blockchain technology system 10 includes a central processing computer component 10A, which can perform any and/or all of the functions described herein as being performed by the central processing computer component 10A and/or the apparatus 100 of FIG. 1, and a distributed ledger and Blockchain technology system component 10B, which can also perform any and/or all of the functions described herein as being performed by the central processing computer component 10A and/or the apparatus 100 of FIG. 1.

In the preferred embodiment the central processing computer component 10A can be a network or server computer. In the preferred embodiment, the central processing computer component 10A can provide control over the apparatus 100 and can perform any of the various processing services and/or functions described herein. The central processing computer component 10A may be a single computer or system of computers and/or may be include a plurality of computers or computer systems which are utilized in conjunction with one another. The central processing computer component 10A, in the preferred embodiment can provide services for any of the other computers and/or computer systems described herein as being associated with any of the individuals, patients, healthcare providers, insurers, payers, brokers, agents, and/or intermediaries, described herein.

With reference once again to FIG. 1, in a preferred embodiment, the apparatus 100 of FIG. 1 also includes a provider communication device 20 or any number of provider communication devices 20 (hereinafter referred to as “provider communication device 20” or “provider computer 20”) which is associated with a healthcare provider such as a healthcare professional, a hospital, a clinic, a pharmacy, a treatment center, a treatment facility, and/or any other provider of services described herein, as well as with any of the various medical specialists, internists, orthopedists, ophthalmologists, cardiologists, hematologists, endocrinologists, oncologists, ears, nose and throat specialists, neurologists, urologists, gastroenterologists, dermatologists, pediatricians, surgeons, surgical specialists, physicians, dentists, endodontists, periodontists, oral surgeons, orthodontists, prosthodontists, dental hygienists, or other dental or oral healthcare providers, psychiatrists, psychologists, optometrists, podiatrists, osteopaths, chiropractors, pharmacists, therapists, physical therapists, respiratory therapists, nurses, healthcare aids, nutritionists, wellness trainers, wellness consultants, or wellness coaches, fitness trainers, fitness consultants, or fitness coaches, and/or any other persons, individuals and/or professionals who can provide healthcare, healthcare-related, wellness and/or wellness-related, and/or fitness and/or fitness-related, services and/or products.

Any healthcare professional, hospital, clinic, pharmacy, treatment center, treatment facility, wellness provider or professional, or fitness provider or professional, and/or any other provider of services, healthcare, wellness, fitness, or otherwise, which is described herein can also be referred to herein as a “provider”. A provider communication device 20 can also be associated with, or can be used by, any research institution, research facility, teaching institution, teaching hospital, college, university, school, or other institution or entity which may perform research, or provide research information or any related information regarding studies, findings, or developments, in the healthcare field or in healthcare-related fields. A provider communication device 20 can also be associated with, or can be used by, any public health department, public health agency, public health facility, or other public health entity, on any one or more of a national level, a country level, a state level, a provincial level, a county level, a city level, a municipal level, or any other level, which is entrusted to provide for the public health and/or to perform services or oversight regarding public health. A provider communication device 20 can also be associated with, or can be used by, any immunization registry or immunization registries. A provider communication device 20 can also be associated with, or can be used by, any source or provider of educational or instructional information, or information regarding instructions or procedures, or links or hyperlinks to same.

Any number or amount of healthcare provider communication devices 20 can be utilized in conjunction with a healthcare provider and/or group of providers. The healthcare provider communication device(s) 20 can communicate with, and can operate in conjunction with, the central processing computer/distributed ledger/Blockchain technology system 10 or the central processing computer component 10A of the same, and/or can communicate with, and can operate in conjunction with, any of the other computers and/or computer systems, and/or communication devices, associated with any of the other individuals and/or entities which utilize and/or operate in conjunction with the present invention.

The apparatus 100 can also include a healthcare insurer or payer communication device or computer 30 (hereinafter referred to as “insurer/payer communication device 30” or “insurer/payer computer 30”) which is associated with a healthcare payer such as a healthcare insurer, insurance company, health maintenance organization, a clinic, and/or any other payer of healthcare services and products described herein. Any number or amount of insurer/payer communication devices 30 can be utilized in conjunction with a healthcare payer and/or group of payers. The insurer/payer communication device(s) 30 can communicate with, and can operate in conjunction with, the central processing computer/distributed ledger/Blockchain technology system 10 or the central processing computer component 10A of the same, and/or can communicate with, and can operate in conjunction with, any of the other computers and/or computer systems, and/or communication devices, associated with any of the other individuals and/or entities which utilize and/or operate in conjunction with the present invention.

The apparatus 100 can also include a user, patient, or individual, communication device or computer or personal monitoring device 40 (which may hereinafter be referred to as “user communication/personal monitoring device 40”). In a preferred embodiment, when used by an individual or patient for personal monitoring purposes, the user communication/personal monitoring device 40 can also be referred to interchangeably as a “personal monitoring device 40”. The user communication/personal monitoring device 40 can also be referred to interchangeably herein as a “user or patient communication device 40”, “user computer 40”, “patient computer 40”, or “user or patient computer or communication device 40”. The user communication/personal monitoring device 40 can be used by or associated with any user of the apparatus 100 or any individual or healthcare patient or client who seeks or who is provided with healthcare and/or related services, products and/or related information. The user communication/personal monitoring device 40 can also be utilized by any individual, party, or entity, who or which may merely utilize the present invention in order to care or another individual or to obtain information of interest.

In a preferred embodiment, the user communication/personal monitoring device 40 can be wired device or can be a wireless device. When used as a personal monitoring device, the user communication/personal monitoring device 40 is a wireless device such as a cellular telephone, a wireless telephone, a smart phone or Smartphone, a watch, a smart watch, a wearable device which can be worn by the individual or patient or attached to an article of clothing or an article of jewelry, or can be an implantable device.

A user communication/personal monitoring device 40 may also be located at public places or locations, such as at kiosks or other publicly available computer or communication devices. Any number or amount of user communication/personal monitoring devices 40 can be utilized in conjunction with a user, patient, group of users, and/or group of patients. The user communication/personal monitoring device 40 can communicate with, and can operate in conjunction with, the central processing computer/distributed ledger/Blockchain technology system 10 or the central processing computer component 10A of the same, and/or can communicate with, and can operate in conjunction with, any of the other computers and/or computer systems, and/or communication devices, associated with any of the other individuals and/or entities which utilize and/or operate in conjunction with the present invention. The user communication/personal monitoring device 40 can also be utilized by any other individual or entity desiring to utilize and/or to obtain information from the apparatus 100. Any number of user communication/personal monitoring devices 40 can be utilized in conjunction with the apparatus 100 of the present invention.

The apparatus 100 can also include an intermediary communication device or computer 50 (hereinafter referred to as “intermediary communication device 50” or “intermediary computer 50”) which is associated with an intermediary, a broker, an agent, and/or any other individual and/or entity, that can utilize the present invention in order to act for and/or on behalf of any other individual, party, or entity, described herein. Any number or amount of intermediary computers 50 can be utilized in conjunction with an intermediary and/or group of intermediaries. The intermediary computer(s) 50 can communicate with, and can operate in conjunction with, the central processing computer/distributed ledger/Blockchain technology system 10 or the central processing computer component 10A of the same, and/or can communicate with, and can operate in conjunction with, any of the other computers and/or computer systems, and/or communication devices, associated with any of the other individuals and/or entities which utilize and/or operate in conjunction with the present invention.

In the preferred embodiment, any of the provider communication device(s) 20, the insurer/payer communication device(s) 30, the user communication/personal monitoring device(s) 40, and/or the intermediary communication device(s) 50, can be any computer or communication device, including, but not limited to, a personal computer, a home computer, a server computer, a network computer, a hand-held computer, a palmtop computer, a laptop computer, a personal communication device, a smart phone or smartphone, a cellular telephone, a wireless telephone, a mobile telephone, a digital television, an interactive television, a digital television, a personal digital assistant, a telephone, a digital telephone, a television, an interactive television, a beeper, a pager, and/or a watch or smartwatch.

Each of the central processing computer component 10A, the provider communication device(s) 20, the insurer/payer communication device(s) 30, the user communication/personal monitoring device(s) 40, and/or the intermediary communication device(s) 50, can transmit information to, as well as receive information from, any of the respective computers or communication devices 10A, 20, 30, 40, and 50, described herein. In this regard, each of the respective computers or communication devices 10A, 20, 30, 40, and 50, can communicate with, process information from, and/or share data and/or information with, each other and/or any other respective computers or communication devices 10A, 20, 30, 40, and 50, described herein and/or utilized in conjunction with the present invention. In this manner, data and/or information transfer between any of the respective computers or communication devices 10A, 20, 30, 40, and 50, can communicate with any other respective computers or communication devices 10A, 20, 30, 40, and 50, in a bi-directional manner.

The central processing computer component 10A, the provider communication device(s) 20, the insurer/payer communication device(s) 30, the user communication/personal monitoring device(s) 40, and/or the intermediary communication device(s) 50, can communicate with one another, and/or be linked to one another, over a communication network, a telecommunication network, a telephone network, a line-connected network, and/or a wireless communication network. Each of the respective computers or communication devices 10A, 20, 30, 40, and 50 can be linked with any other computer or computers directly or indirectly with one another so as to facilitate a direct or indirect bi-directional communication said respective computers. Communications between each of the respective computers or communication devices 10A, 20, 30, 40, and 50 can also involve an e-mail server or e-mail servers in those instances when e-mails are described as being used to transmit or send any of the information, signals, messages, reports, notification messages, or any other communications, described herein, by or between any of the respective computers or communication devices 10A, 20, 30, 40, and 50, or when any of the information, signals, messages, reports, notification messages, or any other communications, described herein, are transmitted by and/or between any of the parties described herein and/or by or between any of the respective computers or communication devices 10A, 20, 30, 40, and 50, or any other computers or communication devices, computer systems, communication network equipment, server computers, etc., or any other devices used or needed in order to facilitate communications or the transmission of any of the herein-described information, signals, messages, reports, notification messages, or any other communications.

The apparatus 100 can also include a healthcare records computer 60 or computers (hereinafter referred to as “healthcare records computer 60” or “healthcare records computer(s) 60) which can be, or can include, a computer or computer system, or any number of computers or computer systems, or a cloud computer system or cloud system. The healthcare records computer 60 can serve to store and house an electronic healthcare record or electronic healthcare files or any number of electronic healthcare records or electronic healthcare files, and/or any, electronic medical records, electronic dental records, electronic pharmacy records, and/or electronic behavioral health records, as well as any personal healthcare records, and/or any other data and/or information regarding and/or contained in any record or record keeping system, software, hardware, or device, or any combination of same or group of same, which can contain healthcare information or healthcare-related information.

In the preferred embodiment, the healthcare records computer 60 can be associated with any provider, insurer, payer, intermediary, insurance exchange, or any user, individual, patient, organization, or entity, who or which utilizes the apparatus 100 and methods of the present invention.

Each healthcare records computer 60 can be utilized to store an electronic healthcare record or electronic healthcare file or any number of electronic healthcare records or electronic healthcare files, and/or any electronic medical records, electronic dental records, electronic pharmacy records, and/or electronic behavioral health records, as well as any personal healthcare records, and/or any other data and/or information regarding and/or contained in any record or record keeping system, software, hardware, or device, or any combination of same or group of same, which can contain healthcare information or healthcare-related information, which can be accessed by the central processing computer/distributed ledger/Blockchain technology system 10 or the central processing computer component 10A of the same, and/or which can be accessed by any of the provider communication device(s) 20, by any of the insurer/payer communication device(s) 30, by any of the user communication/personal monitoring device(s) 40, and/or by any of the intermediary communication device(s) 50, or by any other computer, communication device or other device described herein as being utilized in connection with the apparatus 100 and methods of the present invention. The healthcare records computer 60 can also be utilized to facilitate cloud storage and/or retrieval of any electronic healthcare record(s) or electronic healthcare file(s).

The apparatus 100 can also include an insurance exchange computer 70, or any number of insurance exchange computers 70 which can be utilized to process and store information regarding the selling of healthcare insurance, disability insurance, and life insurance, policies, products, and/or services, to any of the herein-described users, individuals, patients, or entities, who or which utilize the apparatus 100 and method of the present invention. The insurance exchange computer 70 can be utilized to advertise, provide information regarding, sell, and/or maintain records regarding, and process any other information regarding, group insurance as well as individual or family insurance policies, products, or services. The insurance exchange computer 70 can also be utilized to sell automobile, homeowners, business, and/or liability insurance policies, products, or services. The insurance exchange computer(s) 70 can be linked to or with the central processing computer/distributed ledger/Blockchain technology system 10 or the central processing computer component 10A of the same. In a preferred embodiment, any number of insurance exchange computer(s) 70 can be utilized in connection with the apparatus 100 of the present invention.

The apparatus 100 can also include a social networking computer 80. The social networking computer 80 can be linked with, and utilized in connection with, the apparatus 100 and/or any of the respective computers or communication devices 10, 20, 30, 40, 50, 60, 70, and/or 80, so as to allow and/or facilitate integrating the apparatus 100 of the present invention with social networks, social networking, and social media. In a preferred embodiment, for example, the social networking computer 80 can be associated with a social networking company, a social networking website, or social networking entity, website, group, organization, or association.

The social networking computer 80 can be associated with any one or any number of social networking companies, social networking websites, or social networking entities, websites, groups, organizations, or associations. The social networking computer 80 can also provide links to any computers associated with any one or any number of social networking companies, social networking websites, or social networking entities, websites, groups, organizations, or associations. In the preferred embodiment, the social networking computer 80 can perform any and all of the functions performed by any social networking company, a social networking website, or social networking entity, website, group, organization, or association. In a preferred embodiment, any number of social networking computers 80 can be utilized in connection with the apparatus 100 of the present invention.

The apparatus 100 can also include a media computer 90. In the preferred embodiment, the media computer 90 can provide, and be a source of, news information, current events information, healthcare information, healthcare or healthcare-related news, current events, advertisements, and/or marketing information or materials, which can be disseminated via the apparatus 100 of the present invention, In a preferred embodiment, any number of media computers 90, with each being dedicated to providing any number, types, or kinds of, news information, current events information, healthcare information, healthcare or healthcare-related news, current events, advertisements, and/or marketing information or materials, can be utilized in connection with the apparatus 100.

In a preferred embodiment, each of the central processing computer/distributed ledger/Blockchain technology system 10 or the central processing computer component 10A of the same, the provider communication device(s) 20, the insurer/payer communication device(s) 30, the user communication/personal monitoring device(s) 40, the intermediary communication device(s) 50, the healthcare records computer(s) 60, the insurance exchange computer(s) 70, the social networking computer(s) 80, and the media computer(s) 90, can communicate with each other in a bi-directional manner with, and/or can send and/or receive signals, messages, reports, notification messages, alerts, or any other communications or electronic communication transmissions, to, from and/or between, any other, or any number of, other central processing computer/distributed ledger/Blockchain technology systems 10 or the central processing computer components 10A of the same, provider communication devices 20, insurer/payer communication devices 30, user communication/personal monitoring devices 40, intermediary communication devices 50, healthcare records computers 60, insurance exchange computers 70, social networking computers 80, and/or media computers 90.

In a preferred embodiment, each of the central processing computer/distributed ledger/Blockchain technology systems 10 or the central processing computer components 10A, provider communication devices 20, insurer/payer communication devices 30, user communication/personal monitoring devices 40, intermediary communication devices 50, healthcare records computers 60, insurance exchange computers 70, social networking computers 80, and/or media computers 90, can be linked to or with any other central processing computer/distributed ledger/Blockchain technology systems 10 or the central processing computer components 10A, provider communication devices 20, insurer/payer communication devices 30, user communication/personal monitoring devices 40, intermediary communication devices 50, healthcare records computers 60, insurance exchange computers 70, social networking computers 80, and/or media computers 90 via a wired link or line or via a wireless link.

In a preferred embodiment, each of the provider communication devices 20, insurer/payer communication devices 30, user communication/personal monitoring devices 40, intermediary communication devices 50, healthcare records computers 60, insurance exchange computers 70, social networking computers 80, and/or media computers 90, can be connected to or with or linked to or with the central processing computer/distributed ledger/Blockchain technology system 10 and/or to the central processing computer component 10A of the same.

In a preferred embodiment, any and/or all of the signals, messages, reports, notification messages, or any other communications, described herein as being transmitted from any one respective device, computer, or communication device, 10, 20, 30, 40, 50, 60, 70, 80, and/or 90, to another, can be, or can be included in, or be attached to, an e-mail message, an instant messaging message, an electronic transmission, or an electronic data transmission or electronic data interchange, or can be transmitted via and/or in any other data or information transmission, and/or can be transmitted via or using any appropriate or necessary computer(s) or device(s).

In the preferred embodiment, the present invention is utilized on, and/or over, the Internet and/or the World Wide Web. The present invention, in the preferred embodiment, can also utilize wireless Internet and/or World Wide Web services, equipment and/or devices. The central processing computer component 10A of the central processing computer/distributed ledger/Blockchain technology system 10, in the preferred embodiment, has a web site or web sites associated therewith. Each of the other respective computers or communication devices 20, 30, 40, 50, 60, 70, 80, and/or 90 described herein can also have a web site or web sites associated with same.

Although the Internet and/or the World Wide Web is a preferred communication system and/or medium utilized, the present invention, in all of the embodiments described herein, can also be utilized with any appropriate communication network or system including, but not limited to, a communication network or system, a telecommunication network or system, a telephone communication network or system, a cellular communication network or system, a wireless communication network or system, a line or wired communication network or system, a wireless Internet network or system, a wireless World Wide Web network or system, a digital communication network or system, a personal communication network or system, a personal communication services (PCS) network or system, a satellite communication network or system, a broad band communication network or system, a low earth orbiting (LEO) satellite network or system, a public switched telephone network or system, a telephone communication network or system, a radio communication network or system, a cable television network or system, and/or any other communication network or system, and/or any combination of the above communication networks or systems.

In the preferred embodiment, each of the central processing computer/distributed ledger/Blockchain technology systems 10 or the central processing computer components 10A, provider communication devices 20, insurer/payer communication devices 30, user communication/personal monitoring devices 40, intermediary communication devices 50, healthcare records computers 60, insurance exchange computers 70, social networking computers 80, and/or media computers 90, can transmit data and/or information using TCP/IP, as well as any other Internet and/or World Wide Web, and/or communication, protocols.

The apparatus 100 of the present invention can utilize electronic commerce technologies and security methods, techniques and technologies, in any and/or all of the instances of data and/or information processing, and/or data and/or information transmission described herein.

In a preferred embodiment, each of the provider communication devices 20, the insurer/payer communication devices 30, the user communication/personal monitoring devices 40, the intermediary communication devices 50, the healthcare records computers 60, the insurance exchange computers 70, the social networking computers 80, and/or the media computers 90, can be connected to, and/or linked with, the central processing computer/distributed ledger/Blockchain technology system 10, using any suitable and/or appropriate wired connection, wireless connection, or any combination of same, on, via, or using, any suitable and/or appropriate communication network or any combination of communication networks.

In a preferred embodiment, each of the provider communication devices 20, the insurer/payer communication devices 30, the user communication/personal monitoring devices 40 can also include a global positioning device which can be utilized in order to determine the position or location of each of the respective the provider communication devices 20, the insurer/payer communication devices 30, the user communication/personal monitoring devices 40.

With reference once again to FIG. 1, in a preferred embodiment, the apparatus 100 can also include any number of RFID tags/RFID reader system(s) 92, each of which can include any number of RFID tags 92A and RFID reader systems 92B. In a preferred embodiment, the RFID tags/RFID reader system(s) 92 can be utilized in or for any number of a variety ways as described herein and/or otherwise. In a preferred embodiment, the RFID tags/RFID reader system(s) 92 can be connected to, and/or linked with, the central processing computer/distributed ledger/Blockchain technology system 10, using any suitable and/or appropriate wired connection, wireless connection, or any combination of same, on, via, or using, any suitable and/or appropriate communication network or any combination of communication networks.

In a preferred embodiment, any user, individual, patient, caregiver, and/or any healthcare provider, healthcare professional, hospital, clinic, pharmacy, treatment center, treatment facility, and/or any other provider of services, healthcare or otherwise, described herein, who or which uses the apparatus 100 of the present invention, can be assigned a healthcare account or any number of healthcare accounts. Any user, individual, patient, caregiver, and/or any healthcare provider, healthcare professional, hospital, clinic, pharmacy, treatment center, treatment facility, and/or any other provider of services or goods, healthcare, wellness, fitness, or otherwise, described herein can also be assigned any number of types or kinds of healthcare accounts depending upon how they respectively utilize the apparatus 100 of the present invention. As and for an example, an individual who might be a healthcare provider or a physician, can be assigned one or more healthcare accounts for use as a patient who utilizes the apparatus 100 for his or her own healthcare purposes, and/or one of more healthcare accounts for use as a healthcare provider or physician who utilizes the apparatus 100 for providing healthcare services to or for patients.

Any of the herein-described insurers, payers, intermediaries, governmental entities, an/or any other persons or entities who or which utilize the apparatus 100 for his, her, or its, respective purposes can also be assigned one or more healthcare accounts.

In a preferred embodiment, an RFID tag 92A can be assigned to any user, individual, patient, caregiver, and/or any healthcare provider, healthcare professional, hospital, clinic, pharmacy, treatment center, treatment facility, and/or any other provider of services or goods, described herein, healthcare, wellness, fitness, or otherwise, who or which uses the apparatus 100 of the present invention. In a preferred embodiment, the RFID tag 92A can also be assigned to a healthcare account which is assigned to or for the respective user, individual, patient, caregiver, healthcare provider, healthcare professional, hospital, clinic, pharmacy, treatment center, treatment facility, and/or any other provider of services or services, healthcare, wellness, fitness, or otherwise, described herein, who or which uses the apparatus 100 of the present invention.

In a preferred embodiment, the RFID tag 92A can be provided in or as a physical identification card or other physical item or device, and/or can be attached to, or located within, an article of clothing, a watch, a necklace, a bracelet, an ankle bracelet, a ring, an article of jewelry, or any other wearable article or item. In a preferred embodiment, the RFID tag 92A can also be implantable within an individual or patient. In a preferred embodiment, the RFID tag 92A can also be provided in or as a physical identification card, and/or the RFID tag 92A can be attached to, or located on or within, or situated on, a cellular telephone, a smart phone, a Smartphone, a personal digital assistant, a watch, a smartwatch, or on a watch, a smartwatch, a necklace, a bracelet, an ankle bracelet, a ring, an article of jewelry, or any other wearable article or item or any other personal article or item.

In a preferred embodiment, any number of RFID tags 92A can be assigned to or associated with any respective user, individual, patient, caregiver, healthcare provider, healthcare professional, hospital, clinic, pharmacy, treatment center, treatment facility, and/or any other provider of services, healthcare, wellness, fitness, or otherwise, described herein, who or which uses the apparatus 100 of the present invention, and/or to any healthcare account or healthcare accounts associated with the respective user, individual, patient, caregiver, healthcare provider, healthcare professional, hospital, clinic, pharmacy, treatment center, treatment facility, and/or any other provider of services, healthcare, wellness, fitness, or otherwise, described herein.

In a preferred embodiment, an RFID reader system 92B, or any RFID reader or RFID readers of same, can be located at any facility or office, or in a room or venue therein, of any user, individual, patient, caregiver, and/or healthcare provider, healthcare professional, hospital, clinic, pharmacy, treatment center, treatment facility, and/or any other provider of services, healthcare, wellness, fitness, or otherwise, described herein, who or which utilizes the apparatus 100 of the present invention. In another preferred embodiment, an RFID reader system 92B, or any RFID reader or RFID readers of same, can also be located on or implemented with any communication device, computer, computer peripheral device, cellular telephone, smart phone, personal digital assistant, or any other device of or associated with, any user, individual, patient, caregiver, and/or healthcare provider, healthcare professional, hospital, clinic, pharmacy, treatment center, treatment facility, and/or any other provider of services, healthcare, wellness, fitness, or otherwise, described herein, who or which utilizes the apparatus 100 of the present invention.

In a preferred embodiment, the RFID tag/RFID reader system(s) 92, and any RFID tags 92A and any RFID reader system(s) 92B of same, can be utilized for identifying, verifying the identification of, and/or for accessing, any data and/or information, and/or any account(s) or any healthcare account(s) of, associated with, corresponding to, or regarding, any user, individual, patient, caregiver, and/or healthcare provider, healthcare professional, hospital, clinic, pharmacy, treatment center, treatment facility, and/or any other provider of services, healthcare, wellness, fitness, or otherwise, described herein, who or which utilizes the apparatus 100 of the present invention.

With reference once again to FIG. 1, in a preferred embodiment, the apparatus 100 can also include any number of user/patient monitoring systems 94 which can include any number of monitoring devices and/or healthcare measurement tools or devices for detecting, recording, measuring, and/or monitoring, an individual's or patient's heart rate, pulse rate, blood pressure, blood oxygen level(s), blood sugar level(s), or any other biometric measure, measurement, or metric, as well as can include any number of performance measurement devices or tools for detecting, recording, measuring, and/or monitoring, an individual's or patient's physical performance in or during any type or kind of activity, such as, but not limited to, the individual's or patient's rate of speed, rate of acceleration, rate of deceleration, rate of changing direction of movement, distance traveled, speed of travel, and/or any other physical performance measure, measurement, or metric.

In a preferred embodiment, the user/patient monitoring systems 94 can be, or can include, any wearable or non-wearable device or equipment which can be utilized to measure biological, physiological, or any other, data and/or information of, for, or related to, an individual or patient. For example, the user/patient monitoring systems 94 can be, or can include, equipment for measuring pulse rate, heart rate, blood pressure, blood-sugar level, blood oxygen levels or percentages, blood glucose level or blood-sugar level, speed of movement, steps taken, distance traveled, or any other data and/or information regarding the individual or patient, his or her biometric data, and/or a measure of or regarding his or her performance of or in certain physical and/or non-physical tasks or activities.

In a preferred embodiment, the user/patient monitoring systems 94 can be or can include, a thermometer, a digital thermometer, a stethoscope, a heart rate monitor or measurement device, a pulse rate monitor or measurement device, a blood pressure monitor or measurement device, a blood oxygen or percentage oxygen measurement device, a blood pressure measurement device, blood analysis device or machine, a respirator, a respiration monitoring or measurement device, a dialysis machine, a dialysis device, electrocardiograph (EKG) machine or device, electrocephalograph (EEG) machine or device, electromyograph (EMG) machine or device, a magnetic resonance imaging (MRI) machine or device, X-ray machine or device, medical imaging machine or device, thermal imaging machine or device, a heart sound monitor or measurement device, a lung sound monitoring or measurement device, respiration rate monitoring or measurement device, a laparascopic device, an arthroscopic device, a vascular testing device, a catheter device, a cardiac performance testing, monitoring, or measurement device, a pulmonary performance testing, monitoring, or measurement device, a vascular system performance monitoring or measurement device, a vascular system testing, monitoring, or measurement device, a metabolism monitoring or measurement device, a sonogram imaging device, a sonogram measurement device, a sonograph device, an optical response device, an optical response measurement device, an intravenous device, an arterial blood pressure measurement or monitoring device, a respiration rate measurement or monitoring device, an ultrasound imaging device, an ultrasound measurement device, a CAT SCAN device, a PET scan device, an optical metabolism measurement or monitoring device, a radiotelemetric device, a doppler medical device, a mammogram device, a carbon dioxide detection or measurement device, a carbon monoxide detection or measurement device, a transvascular impedance measurement or monitoring device, an ultrasonic imaging device, a bone conduction device, a brain function scan analyzer device, external pulse cardiac monitoring or measurement device, a fetal heart rate measurement, monitoring, or probing, device, an endotracheal cardiac monitoring device, a finger tip blood pressure monitoring device, a psychological monitoring device, a surgical instrument, a vital signs measurement device, ear pressure regulating device, phonocardiograph device, acoustic aneurysm detector device, blood oxygen detection device, esophageal probing device, ultrasonic probing device, ausculoscope, vital signs monitoring system, heart activity monitoring device, pulmonary activity monitoring device, sphygmomanometer, esophageal stethoscope, venous pressure measuring device, differential doppler device, physiological data measuring device, body tissue movement device, breathalyzer device, camera probing device, microscopic camera probing device, any scope having a camera and/or a light, a laparoscopic device, a laparoscopic camera device, a camera used in or associated with equipment for performing visual probing of the body or any portions, parts, or organs, thereof, such as, but not limited to, a colonoscopy, an endoscopy, an esophagoscopy, a gastroscopy, or any devices used in laparoscopic surgery or other surgeries, an ingestible or implantable pill, capsule, or device, containing a camera and/or a light and equipment or circuitry for transmitting information as well as images to equipment inside or outside the body, and/or any other bio-metric or physiological data measuring device(s) and/or data acquisition device.

In a preferred embodiment, the user/patient monitoring systems 94 can also be or can also include, a gyroscope, an accelerometer, a decelerometer, a magnetometer, an RFID tag(s) 92, a thermometer for measuring temperature of the environment, a device for measuring body temperature, a device for measuring speed of movement, a device for measuring distance traveled, or any other device or devices which can measure and record information regarding three-dimensional movements of the individual or patient. In a preferred embodiment, and depending upon the application, the user/patient monitoring systems 94 can also be, or include, any devices or equipments which are needed, required, or desired, for measuring, monitoring, and/or tracking individual or patient activity or performance using any type or kind of optical-based camera (OBC) tracking systems, local positioning system (LPS) tracking systems, and/or global positioning system/global navigation satellite system (GPS/GNSS) tracking systems, and/or any other tracking system, which are or can be utilized to track players or athletes during sporting or athletic competitions or training.

In a preferred embodiment, the respective optical-based camera (OBC) tracking systems, local positioning system (LPS) tracking systems, and/or global positioning system/global navigation satellite system (GPS/GNSS) tracking systems, which are utilized to track individual or patient activity or performance can also utilize or include any of the herein-described RFID tags/RFID reader system(s) 92.

In a preferred embodiment, the user/patient monitoring systems 94 can be utilized in or for any number of a variety ways as described herein and/or otherwise. In a preferred embodiment, the user/patient monitoring systems 94 can be connected to, and/or linked to or with, the central processing computer/distributed ledger/Blockchain technology system 10, using any suitable and/or appropriate wired connection, wireless connection, or any combination of same, on, via, or using, any suitable and/or appropriate communication network or any combination of communication networks.

In a preferred embodiment, the user/patient monitoring systems 94, or any device or equipment of the same, can also be connected to, integrated in or within, and/or linked to or with, any user communication/personal monitoring device(s) 40, using any suitable and/or appropriate wired connection, wireless connection, or any combination of same, on, via, or using, any suitable and/or appropriate communication network or any combination of communication networks. In a preferred embodiment, for example, any of device or component, which is one of or included in the user/patient monitoring systems 94, can be linked with a user communication/personal monitoring device 40 using Bluetooth technology, WiFI technology, and/or any other wireless communication technology or wireless protocol. As and for at least one example, contacts, electrodes, sensors, or leads, of a respective EKG device, thermometer, blood pressure monitor, heart rate monitor, blood sugar monitor, a blood oxygen monitor, or other or respective software application, can be connected to a user communication/personal monitoring device 40, which can be a smartphone or a smartwatch, via a Bluetooth or other suitable wireless connection.

In a preferred embodiment, the central processing computer/distributed ledger/Blockchain technology system 10, or the central processing computer component 10A of same, can be specially programmed to perform all of the functions and functionalities described as being performed by the respective central processing computer or by the respective central processing computer/distributed ledger/Blockchain technology system, or the central processing computer component of same, which is/are described in each of U.S. patent application Ser. No. 16/202,448, U.S. patent application Ser. No. 14/638,934, U.S. Provisional Patent Application Ser. No. 61/971,225, U.S. patent application Ser. No. 17/224,838, U.S. patent application Ser. No. 18/233,321, U.S. patent application Ser. No. 18/225,734, U.S. patent application Ser. No. 18/223,700, U.S. Pat. Nos. 7,464,040, 7,490,048, and 10,571,284, the subject matter and teachings of all of which are herein incorporated by reference in their entirety as if to be fully restated herein.

In a preferred embodiment, the user communication/personal monitoring device 40 can be specially programmed to perform all of the functions and functionalities described as being performed by the respective user communications devices or the respective patient communication devices which is/are described in each of U.S. patent application Ser. No. 16/202,448, U.S. patent application Ser. No. 14/638,934, U.S. Provisional Patent Application Ser. No. 61/971,225, U.S. patent application Ser. No. 17/224,838, U.S. patent application Ser. No. 18/233,321, U.S. patent application Ser. No. 18/225,734, U.S. patent application Ser. No. 18/223,700, U.S. Pat. Nos. 7,464,040, 7,490,048, and 10,571,284, the subject matter and teachings of all of which are herein incorporated by reference in their entirety as if to be fully restated herein.

In a preferred embodiment, the provider communication device 20 can be specially programmed to perform all of the functions and functionalities described as being performed by the respective provider communication devices or the respective provider computers which is/are described in each of U.S. patent application Ser. No. 16/202,448, U.S. patent application Ser. No. 14/638,934, U.S. Provisional Patent Application Ser. No. 61/971,225, U.S. patent application Ser. No. 17/224,838, U.S. patent application Ser. No. 18/233,321, U.S. patent application Ser. No. 18/225,734, U.S. patent application Ser. No. 18/223,700, U.S. Pat. Nos. 7,464,040, 7,490,048, and 10,571,284, the subject matter and teachings of all of which are herein incorporated by reference in their entirety as if to be fully restated herein.

In a preferred embodiment, any and/or all of the various operations, transactions, functions, and/or functionalities, described herein as being performed by the apparatus 100 in the preferred embodiment of FIG. 1 can also be performed by either the central processing computer component 10A of the central processing computer/distributed ledger/Blockchain technology system 10 and/or by the distributed ledger and Blockchain technology system component 10B, or both. In a preferred embodiment, for example, any and/or all transactions involving any storing of any of the herein-described data and/or information, and/or any data and/or information regarding any video messages, video clips, audio messages, audio clips, video call(s), and/or remote office visit(s), and/or any video call reports and/or any of the remote or virtual office visit reports or any remote or distance examination reports, in the individual's or the patient's electronic healthcare record, file, or history, can be performed by the central processing computer component 10A of the central processing computer/distributed ledger/Blockchain technology system 10, and/or by the distributed ledger and Blockchain technology system component 10B, and/or by both. In a preferred embodiment, any data and/or information regarding any transactions processed via the distributed ledger and Blockchain technology system component 10B can also be stored in the central processing computer component 10A.

In a preferred embodiment, transactions which are to be processed by the central processing computer component 10A and by the distributed ledger and Blockchain technology system component 10B can be pre-selected or pre-programmed into the central processing computer/distributed ledger/Blockchain technology system 10, and can be re-programmed at any time. In a preferred embodiment, the use of the distributed ledger and Blockchain technology system component 10B can be utilized to provide added security for the individual's or the patient's respective electronic healthcare record, file, or history, and can also be utilized to prevent healthcare identity theft involving the individual or patient.

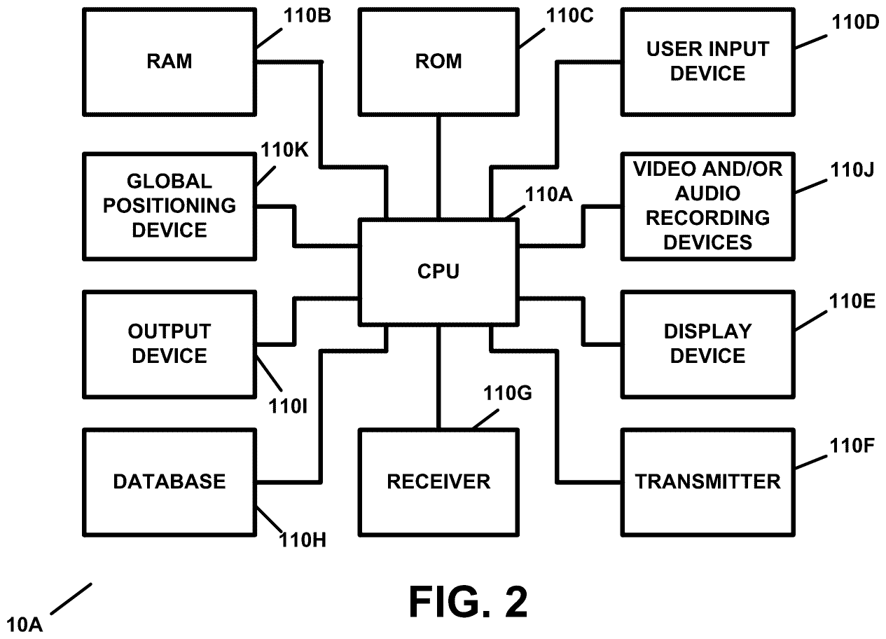

FIG. 2 illustrates the central processing computer component 10A of the central processing computer/distributed ledger/Blockchain technology system 10 of FIG. 1, in block diagram form. The central processing computer component 10A, in the preferred embodiment, is a network computer or computer system, or any other communication device which can provide the functionality of, and which can be utilized as a central processing computer such as an Internet server computer and/or a web site server computer. In the preferred embodiment, the central processing computer component 10A includes a central processing unit or CPU 110A, which in the preferred embodiment, is a microprocessor. The CPU 110A may also be a microcomputer, a minicomputer, a macro-computer, and/or a mainframe computer, depending upon the application.

The central processing computer component 10A also includes a random access memory device(s) 110B (RAM) and a read only memory device(s) 110C (ROM), each of which is connected to the CPU 110A, a user input device 110D, for entering data and/or commands into the central processing computer component 10A, which includes any one or more of a keyboard, a scanner, a user pointing device, such as, for example, a mouse, a touch pad, and/or an audio input device and/or a video input device, a microphone or audio recording device, a camera or a video recording device, and/or any device, electronic and/or otherwise which can be utilized for inputting and/or entering healthcare data and/or information, for example pulse rate monitors, blood pressure monitors, electrocardiograms, glucose monitors, blood-sugars monitors, blood oxygen or percentage oxygen measurement devices, etc., if desired, which input device(s) are also connected to the CPU 110A. The central processing computer component 10A also includes a display device 110E for displaying data and/or information to a user or operator.

The central processing computer component 10A also includes a transmitter(s) 110F, for transmitting signals and/or data and/or information to any other central processing computer component(s) 10A, and/or to any one or more of the provider communication devices 20, the insurer/payer communication devices 30, the user communication/personal monitoring devices 40, the intermediary communication devices 50, the healthcare records computers 60, the insurance exchange computers 70, the social networking computers 80, and/or the media computers 90, or any other individual computer(s), which may be utilized in conjunction with the present invention. The central processing computer component(s) 10A also includes a receiver 110G, for receiving signals and/or data and/or information from any other central processing computer component(s) 10A, and/or from any one or more of the provider communication devices 20, the insurer/payer communication devices 30, the user communication/personal monitoring devices 40, the intermediary communication devices 50, the healthcare records computers 60, the insurance exchange computers 70, the social networking computers 80, and/or the media computers 90, or any other individual computer(s), which may be utilized in conjunction with the present invention.

The central processing computer component 10A also includes a database(s) 110H which contains data and/or information pertaining to the individuals, patients, providers, payers, insurers, and intermediaries, who or which are serviced by the present invention and/or who or which utilize the present invention.

In a preferred embodiment, the database(s) 110H of the central processing computer component 10A contains and/or all data, information, software programs, software algorithms, software applications, which are needed to required for performing any and/or all of the functions, functionalities, processing routines, and/or operations described herein as being performed by the central processing computer component 10A and/or by the apparatus 100.

In a preferred embodiment, the database(s) 110H of the central processing computer component 10A also contains any and/or all artificial intelligence (AI) software programs, software algorithms, software applications, machine language software programs, software algorithms, software applications, and/or natural language software programs, software algorithms, software applications, which are needed to required for performing any and/or all of the functions, functionalities, processing routines, and/or operations described herein as being performed by the central processing computer component 10A and/or by the apparatus 100.

In a preferred embodiment, the database 110H of the central processing computer component 10A also contains any and/or all of the data, information, software programs, software algorithms, software applications, which is/are described as being stored in any the respective databases in, of, or associated with, any of the various and respective central processing computers, computers, and/or communications devices which are disclosed and described in each of U.S. patent application Ser. No. 16/202,448, U.S. patent application Ser. No. 14/638,934, U.S. Provisional Patent Application Ser. No. 61/971,225, U.S. patent application Ser. No. 17/224,838, U.S. patent application Ser. No. 18/233,321, U.S. patent application Ser. No. 18/225,734, U.S. patent application Ser. No. 18/223,700, U.S. Pat. Nos. 7,464,040, 7,490,048, and 10,571,284, the subject matter and teachings of all of which are herein incorporated by reference in their entirety as if to be fully restated herein.

The database 110H can contain any one or any number of electronic healthcare records (EHRs), which electronic healthcare records definition includes electronic healthcare records or electronic healthcare files, electronic medical records or electronic healthcare files (EMRs), electronic dental records or files (EDRs), electronic pharmacy records or files (EPRs), electronic behavioral health records or files (EBHRs), as well as any personal health records or files (PHRs). The database 110H can contain any one or any number of electronic healthcare records (EHRs), which electronic healthcare records definition includes electronic healthcare records or electronic healthcare files, electronic medical records or electronic healthcare files (EMRs), electronic dental records or files (EDRs), electronic pharmacy records or files (EPRs), electronic behavioral health records or files (EBHRs), as well as any personal health records or files (PHRs) for each user, individual, or patient, who utilizes the apparatus and methods of the present invention. The database 110H can contain any one or any number of electronic healthcare records (EHRs), which electronic healthcare records definition includes electronic healthcare records or electronic healthcare files, electronic medical records or electronic healthcare files (EMRs), electronic dental records or files (EDRs), electronic pharmacy records or files (EPRs), electronic behavioral health records or files (EBHRs), as well as any personal health records or files (PHRs), any number of users, individuals, or patients.

The database 110H can also contain and/or include, for each individual or patient, and/or in each respective electronic healthcare record or electronic healthcare file, electronic medical record or electronic healthcare file (EMR), electronic dental record or file (EDR), electronic pharmacy record or file (EPR), electronic behavioral health record or file (EBHR), as well as any personal health record or file (PHR), for the respective individual or patient, information regarding any and/or all immunizations previously provided to the individual or patient, information regarding any and/or all immunizations not yet, or not previously, provided to the individual or patient, information regarding any and/or immunizations needed by the individual or patient, and/or any information regarding any previous or current diagnosis, or state of health, regarding any disease, viral infection, bacterial infection, sickness, infirmity, weakness, or ailment, of or regarding the individual or patient, with which the individual or patient has been diagnosed, or which the individual or patient is currently experiencing or enduring.

The database 110H can also contain and/or include, for each user, individual, or patient, a digital certificate or any number of digital certificates which can contain information regarding any and/or all immunizations previously provided to the individual or patient, information regarding any and/or all immunizations not yet, or not previously, provided to the individual or patient, information regarding any and/or immunizations needed by the individual or patient, and/or any information regarding any previous or current diagnosis, or state of health, regarding any disease, viral infection, bacterial infection, sickness, infirmity, weakness, or ailment, of or regarding the individual or patient, with which the individual or patient has been diagnosed, or which the individual or patient is currently experiencing or enduring.

The database 110H can also contain and/or include passport information or other official identification information for any user, individual, or patient. In a preferred embodiment, the passport information or other official identification information for any user, individual, or patient, can also include or contain, or include as an attachment, a digital certificate or any number of digital certificates which can contain information regarding any and/or all immunizations previously provided to the individual or patient, information regarding any and/or all immunizations not yet, or not previously, provided to the individual or patient, information regarding any and/or immunizations needed by the individual or patient, and/or any information regarding any previous or current diagnosis, or state of health, regarding any disease, viral infection, bacterial infection, sickness, infirmity, weakness, or ailment, of or regarding the individual or patient, with which the individual or patient has been diagnosed, or which the individual or patient is currently experiencing or enduring.

The database 110H can also contain any links or hyperlinks to any of the above electronic healthcare records or electronic healthcare files, electronic medical records or files, electronic dental records or files, electronic pharmacy records or files, electronic behavioral health records or files, and/or personal health records or files, which may be located or stored on any computer or computers external from the central processing computer component 10A, for any number of users, individuals, or patients.

Each of the herein-described electronic healthcare records, electronic medical records, electronic dental records, electronic pharmacy records, and/or electronic behavioral health records can also contain a personal health record portion or a personal health record. The apparatus 100 and method of the present invention can store a personal health record for each individual or patient. The personal health record can be stored in each individual's or patient's respective electronic healthcare record(s), electronic medical record(s), electronic dental record(s), electronic pharmacy record(s), and/or electronic behavioral health record(s), as well as in any user or patient communication device 40. The personal healthcare record can be updated as described herein or in any other appropriate manner.

The database 110H can also contain a look-up table or look-up tables and/or data and/or information relating thereto, for identifying and locating a respective electronic healthcare record(s), an electronic medical record, an electronic dental records, an electronic pharmacy record, an electronic behavioral health record, or a personal health records, for or associated with any user, individual, patient, or person who may utilize the apparatus 100 of the present invention. In this regard, the database 110H can store any healthcare data and/or information or healthcare-related data and/or information for any user, individual, patient, or person who may utilize the apparatus 100 of the present invention, whether that information is stored in the central processing computer component 10A, the database 110H, or in of the any provider communication devices 20, insurer/payer communication devices 30, user communication/personal monitoring devices 40, intermediary communication devices 50, healthcare records computers 60, insurance exchange computers 70, social networking computers 80, and/or media computers 90, regardless of where these computers or communications devices are located and regardless of what data and/or information is stored on same.

In the preferred embodiment, the database 110H contain data and/or information for locating, retrieving, storing, or providing a comprehensive healthcare record of healthcare file for any individual, user, patient, or person. The database 110H can also contain any necessary data or information for locating, identifying, retrieving, storing, any information from any of the various types of kinds of electronic healthcare records or electronic healthcare files regarding of the type, kind, and/or vendor or supplier of same, regardless of where these electronic healthcare records or electronic healthcare files are located or stored. In a preferred embodiment, the database 110H can also contain an index or other information for identifying data fields in the respective databases of the various respective electronic healthcare records for enabling the central processing computer component 10A to identify data and/or information in each of the respective electronic healthcare records or electronic healthcare files.

The database 110H can also contain any data and/or information for allowing any herein-described user, individual, patient, person, provider, payer, intermediary, or entity, to access or obtain any of the herein-described data and/or information, wherever such data and/or information is located or stored, as well as to perform any function described herein as being capable of being performed by the apparatus 100 of the present invention.

The database 110H can also contain any data and/or information for integrating, linking to or with, and/or accessing data and/or information stored or located in, any number, type, or kind, of any of the herein-described electronic healthcare records or electronic healthcare files, electronic medical records or files, electronic dental records or files, electronic pharmacy records or files, electronic behavioral health records or files, as well as any personal health records or files.

The database 110H can contain any of the herein-described data and/or information for any number of individuals, users, patients, or persons. The database 110H can also contain data and/or information regarding a name, relationship, and authorization for any parent, relative, legal guardian, caregiver, healthcare proxy holder, or any person, individual, or entity, who or which may be responsible for caring for, accessing, updating, or maintaining healthcare records or files for, making decisions for, or making healthcare-related decisions for, taking actions for, providing care for, or performing any task or activity for, any other individual(s), user(s), patient(s), or person(s). For example, the database 110H can, for a child, contain information regarding the child's parent or legal guardian, and for the database 110H, for an elderly person of any other person, contain information regarding the person's child, caregiver, healthcare proxy holder, or any other authorized person or entity, or for individuals who belong to a school or club's athletic program, or a sports team, a club, a social or other organization, the database 110H can contain information regarding a coach, team doctor, trainer or other authorized individual.

The database 110H can contain any and/or all of the information needed and/or required in order to perform any and/or all of the functions, services and/or operations described herein as being performed by the central processing computer component 10A or the apparatus 100 of the present invention. In this regard the database 110H can contain data and/or information regarding patient name, patient identification information, patient social security number or other identification information, date of birth, sex, gender, race, nationality, ethnicity, spoken language, preferred language, other languages, religion or religious affiliation, next-of-kin, relatives, emergency contact person, doctors or providers, therapists, nutritionists, insurance or payer information, group insurance information, group health insurance information, life insurance information, disability insurance information, patient address, phone number, e-mail and/or other contact information, medical history, psychological history, dental history, family history, family medical, psychological, and/or dental history, past and/or current or active symptoms, past and/or current or active diagnoses, past and/or current or active treatments or treatment plans, care management plan or plans, allergies, active or current medications or prescription drugs, active or current medication allergies, past, active, or current allergies, smoking status, insurance coverage, insurance co-payment and/or deductible information, co-payment information regarding any payer or insurer insurance policy or payment plan, deductible information regarding any payer or insurer insurance policy or payment plan, benefit eligibility information, insurance information, insurance claim procedures, insurance claim forms, doctor or provider appointment schedules, past treatments, past diagnosis, symptoms, insurance claim forms, employer information, lifestyle information, treatment plans, treatment progress, broker/agent/intermediary information, education information, age, sex, marital status, employee benefits information, types or services and/or treatments needed, and any other data and/or information regarding the patient which would be needed and/or desired in order to perform any and/or all of the functions, services and/or operations described herein.

The database 110H can also contain provider office visit summaries, hospital discharge summaries, hospital discharge instructions, and/or any examination, treatment, testing, or procedure, summary, and any instructions, treatment plan or instructions, care management plan or instructions, or any other information which can be provided to any individual, patient, provider, payer, insurer, third party or intermediary, or caregiver, described herein as using the apparatus 100 and method of the present invention, or described herein as being able to receive services, provide services, or otherwise use or be serviced by, the apparatus 100 and method of the present invention.

In the case of deceased patients or individuals, the database 110H can contain information regarding date of death, place of death, preliminary cause of death, and/or final determination of cause of death. The database 110H can also contain information regarding any other information regarding a deceased patient or individual and can also contain information regarding final resting place.

The database 110H can also contain, for each patient or individual, information regarding dates of appointments or services with or from healthcare providers or other providers, dates of laboratory tests, dates of treatments, operations, or procedures, dates of doctor visits, therapy sessions, vital signs, healthcare chart changes, or information regarding height, weight, blood pressure, heart rate, pulse rate, body-mass index, growth charts, and/or any other related or pertinent data or information.

The database(s) 110H can also contain healthcare and/or medical video, image, and/or audio, and/or text, data and/or information, and/or surgical video, image, and/or audio, and/or text, data and/or information, and/or dental video, image, and/or audio, and/or text, data and/or information, such as, for example, x-rays, Magnetic Resonant Images (MRI), CAT scans, digital x-ray files, digital Magnetic Resonant Imaging (MRI) files, digital CAT scan files, and/or any other video, imaging, and/or audio, healthcare data and/or information which can be utilized by healthcare providers, payers, intermediaries, patients, and/or other users of the present invention. In this manner, the present invention can facilitate the availability of any of the above-described video, image, and/or audio, data and/or information in a network environment. For example, a medical specialist can have access to, and/or review, an MRI or a CAT scan for a patient, from any location and at any time.

The database 110H can also contain data and/or information regarding providers including provider name, provider social security number or identification number, type of professional or service provider, address, phone number, fax number, e-mail and/or other contact information, experience, specialties, insurances accepted, schedule of charges, financial account identification information, resume information, education, work experience, claim forms, appointment schedules, procedures performed, and/or any other data and/or information concerning the providers for providing any and/or all of the functions, services, and/or operations described herein as being performed by the present invention.

The database 110H can also contain data and/or information regarding all possible fields of medicine, surgery, psychiatry, psychology, dentistry, oral surgery, optometry, podiatry, physical therapy, respiratory therapy, hypnosis, osteopathy, nutrition, wellness, and/or any other possible healthcare fields and/or subject matter which can possibly by utilized in the processing and/or operation of the present invention.

The database 110H can also contain information on illnesses, symptoms, diseases and/or sicknesses, theories, scientific theories, research data and/or information, diagnosis information, treatment information, treatment plans, treatment processes, treatment progresses, treatment interactions, side effects, expected treatment results, treatment providers, treatment durations, treatment costs, pre-treatment information, post-treatment information, treatment monitoring information, statistical information regarding diagnoses, treatments, treatment success rates, treatment failure rates, treatment centers, therapy plans, therapy success rates, therapy failure rates, treatment procedures, medications treatments, non-medication treatments, healthcare institutions, treatment evaluating criteria, treatment mistakes and/or mishaps, indicators of mistakes and/or mishaps, corrective actions, links to providers, links to treatment centers or institutions, reimbursement rates, nutrition information, diet information, exercise information, exercise routines, treatment options, healthcare advise, wellness advice, preventive care, preventive procedure, health maintenance, drug and medication information, drug interaction information, video information, including video files or clips and other information, regarding illnesses, diseases, treatments and follow-up care, audio information, including audio files or clips and other information, regarding illnesses, diseases, treatments and follow-up care, treatment and/or procedure information and/or narratives, treatment analysis, diagnosis analysis, diagnosis monitoring, diagnosis confirmation and/or checking, and/or other information for providing the herein-described functions, services, and/or operations.

The database 110H can also contain information regarding the insurance companies and payers described herein, including, but not limited to, payer name, address, phone number, fax number, e-mail address, identification number(s), coverage types, policies and/or coverages provided, reimbursement rates, patients and/or providers serviced and/or covered by the payer, policy information, claim forms, claim procedures, claim status, claim processing information, claim submission procedures and policies, reasonable and customary charges, co-payment information, pre-approval information and/or procedures, claim form information, electronic form claim forms, insurance and/or coverage requirements, guidelines, and/or triggering events, covered procedures and/or treatments, uncovered procedures and/or treatments, claim approval information, claim approval history, claim approval statistics, claim rejection or denial information, claim rejection or denial history, claim rejection or denial statistics, financial account information, network provider information, network patient information, claim statistics, preventative care and/or benefits information, benefits information, benefits request information and/or claim forms, claim submission information, claim processing information, claim status information, payment information and statistics, and/or any other data and/or information regarding and/or related to payers which are needed and/or desired for providing any and/or all of the functions, services, and/or operations described herein.

The database 110H can also contain data and/or information regarding the brokers, agents and/or intermediaries described herein, including, but not limited to, intermediary name, address, phone number, fax number, e-mail address, clients, patients services, insurance policies, policy information, policy quote information, policy proposal information, any and/or all of the above information described herein regarding patients, providers, payers, etc., which may be of interest to the intermediaries described herein which may be useful and/or beneficial to the intermediaries in providing any of the functions, services, and/or operation described herein.

The database 110H can also contain contact information such as phone numbers, fax numbers, pager numbers, beeper numbers, e-mail addresses, hyperlinks to, and/or any other information which can facilitate contact between any of the parties described herein. The database 110H also includes electronic signature data and/or information for any of the parties, patients, providers, payers, and/or intermediaries, described herein for facilitating transactions, claim submissions, financial transactions, etc., by and/or between any of the above patients, providers, payers, and/or intermediaries.

The database 110H can also contain and notes, comments, or messages, which can be provided by any user, individual, patient, caregiver, provider, insurer, payer, third party, intermediary, or any other person or entity who or which utilizes the apparatus 100 and method of the present invention.

The database 110H can also contain any number of electronic healthcare records, electronic medical records, electronic dental records, electronic pharmacy records, electronic behavioral health records, and/or links to other electronic healthcare records, electronic medical records, electronic dental records, electronic pharmacy records, electronic behavioral health records which might be located in healthcare records computers 60 or other computers or computer systems located remote from the central processing computer component 10A. Any and/or all of these electronic healthcare records, electronic medical records, electronic dental records, electronic pharmacy records, electronic behavioral health records can contain data and/or information and/or link or hyperlinks to data and/or information regarding any individuals, patients, his or her caregivers, children, parent, relatives, friends, social networking friends, connections, of followers, providers, insurers or payers.

The database 110H can also contain any and/or all information regarding the data and/or information fields in each electronic healthcare record, electronic medical record, electronic dental record, electronic pharmacy record, and/or electronic behavioral health record, and/or personal healthcare record, and can also contain any and/or all information needed or required for mapping common or like data fields for each and information or links or hyperlinks for accessing like data or like information fields in each of the different electronic healthcare records, electronic medical records, electronic dental records, electronic pharmacy records, electronic behavioral health records, and/or personal healthcare records.

The database 110H can also contain any and/or all information of, for, and/or relating to any and/or all of the various electronic healthcare records, electronic medical records, electronic dental records, electronic pharmacy records, electronic behavioral health records, and/or personal healthcare records, which are available in the marketplace, commercially and/or otherwise, along with any and/or all data and/or information fields in and for each respective electronic healthcare record, electronic medical record, electronic dental record, electronic pharmacy record, and/or electronic behavioral health record, and/or personal healthcare record,

The database 110H can also contain any and/or all information regarding the data and/or information fields in and for each respective electronic healthcare record, electronic medical record, electronic dental record, electronic pharmacy record, and/or electronic behavioral health record, and/or personal healthcare record.

The data and/or information in the database 110H can also include links to any other information, information sources, news sources, and/or other information and/or data which can or may be utilized by the present invention and/or by any of the patients, providers, payers, intermediaries and/or any other users of the present invention.

The database 110H can also contain data and/or information regarding healthcare news, healthcare developments, healthcare discoveries, etc., for and including the medical field, surgical field, psychological field, dental field, nutrition field, fitness field, etc., and/or any other healthcare field or fields.

The database 110H, in the preferred embodiment, can also contain video and/or audio files which can be utilized for training of healthcare professionals as well as for providing general information to any user of the present invention. In this manner, and as will be described hereinbelow, the apparatus 100 can be utilized as a simulator for providing training in medical diagnosing, medical training, surgical training, psychiatric training, psychological training, dental training, oral surgery training, therapist training, and/or for training any of the healthcare providers described herein and/or envisioned.

For example, the present invention can be utilized to provide a medical doctor with a set of symptoms, evaluate the diagnosis and treatment prescribed and provide follow-up patient conditions which may or may not call for the medical doctor to re-evaluate his or her diagnosis and/or treatment. In a similar fashion, the present invention can be used for training and continuing education and training for any of the healthcare providers described herein and/or otherwise envisioned utilizing the present invention.

The database 110H can also contain data and/or information restricting access by any of the providers, payers, patients, intermediaries, and/or other users, to any of the data and/or information stored in the database 110H.

The database 110H can also contain information correlating symptoms and/or conditions with diagnoses, prognoses, and/or treatments, treatment methods, procedures, etc. The database 110H also contains any and/or all information needed and/or desired for facilitating the processing of symptoms, conditions, medical histories, family histories, and other information, in order to arrive at diagnoses and/or prognoses, treatments, prescriptions, procedures and/or any other healthcare and/or healthcare-related information.

The database 110H can also contain information regarding medical records, medical charts, x-rays, MRIs, and/or any other information, described herein and/or otherwise, which can be associated with an individual's or patient's medical history, medical record, or medical file, for any individual or patient who utilizes the apparatus 100 and method of the present invention.

The database 110H can also contain information regarding surgical records, surgical charts, x-rays, MRIs, and/or any other information, described herein and/or otherwise, which can be associated with an individual's or patient's surgical history, surgical record, or surgical file, for any individual or patient who utilizes the apparatus 100 and method of the present invention.

The database 110H can also contain information regarding dental records, dental charts, x-rays, and/or any other information, described herein and/or otherwise, which can be associated with an individual's or patient's dental history, dental record, or dental file, for any individual or patient who utilizes the apparatus 100 and method of the present invention.

The database 110H can also contain information regarding a prescription drug record, file, or account, for an individual or patient for any individual or patient who utilizes the apparatus 100 and method of the present invention.

The database 110H can also contain information regarding a healthcare spending account or healthcare spending accounts for any individual or patient, or for any of the individuals or patients, described herein or otherwise, who utilize the apparatus 100 and method of the present invention. For example, the healthcare spending account is or can be an account which can be a deposit account, a credit account, a debit account, an electronic money or an electronic cash account, or any combination of same, which can be held by any individual, patient, employer, bank, credit union, financial institution, investment institution, healthcare insurer, healthcare payer, or any other third party, and which can be used as a source of funds for payment for any type of healthcare service or healthcare product which is provided to, or administered to, or sought by, any individual or patient who utilizes the apparatus 100 and method of the present invention. In a preferred embodiment, the healthcare spending account can be used by the individual or patient to pay for any healthcare service, treatment, procedure, or product, at any time a payment is desired to be made for same.

The database 110H can also contain and/or include, for each individual, patient, caregiver, healthcare provider, or any other provider, healthcare insurer or healthcare payer, or any intermediary, who or which utilizes the apparatus 100 of the present invention, information regarding, or a link or a hyperlink, to any information regarding a telephone number or an IP address, or other contact information, for to regarding any communication device or computer which can be utilized by the respective individual, patient, caregiver, healthcare provider, or any other provider, healthcare insurer or healthcare payer, or any intermediary, in making and/or in engaging in any of the video calls, video chat sessions, or videoconferences, described herein.

The database 110H can also contain and/or include, for each healthcare provider who or which utilizes the apparatus 100 of the present invention and who or which desires to engage in video calls, video chat sessions, or videoconferences, and/or in remote or virtual office visits or in remote or distance examinations, any data and/or information or a link or hyperlink to any data and/or information regarding or containing the healthcare provider's work schedule(s) as well any information for allowing any individual, patient, or caregiver, to schedule and/or conduct a video call, a video chat session, or a videoconference, and/or a remote or a virtual office visit or a remote or a distance examination, with the healthcare provider.

The database 110H can also contain and/or include, for each healthcare provider who or which utilizes the apparatus 100 of the present invention and who or which desires to engage in video calls, video chat sessions, or videoconferences, any data and/or information or a link or hyperlink to any data and/or information regarding or containing the healthcare provider's work schedule(s) as well any information for allowing any healthcare insurer or healthcare payer, or any intermediary, to schedule and/or conduct a video call, a video chat session, or a videoconference, with the healthcare provider.

The database 110H can also contain and/or include, for each healthcare insurer or healthcare payer who or which utilizes the apparatus 100 of the present invention and who or which desires to engage in video calls, video chat sessions, or videoconferences, any data and/or information or a link or hyperlink to any data and/or information regarding or containing the healthcare insurer's or healthcare payer's work schedule(s) as well any information for allowing any individual, patient, or caregiver, or any healthcare provider, or any intermediary, to schedule and/or conduct a video call, a video chat session, or a videoconference, with the healthcare insurer or the healthcare payer.

The database 110H can also contain and/or include, for each intermediary who or which utilizes the apparatus 100 of the present invention, and who or which desires to engage in video calls, video chat sessions, or videoconferences, any data and/or information or a link or hyperlink to any data and/or information regarding or containing the intermediary's work schedule(s) as well any information for allowing any individual, patient, or caregiver, or any healthcare provider, or any healthcare insurer or healthcare payer, to schedule and/or conduct a video call, a video chat session, or a videoconference, with the intermediary.

The database 110H can also contain and/or include, for each healthcare provider who or which has registered with the apparatus 100 in order to engage in video calls, video chat sessions, or videoconferences, in order to provide remote or virtual office visits or in order to provide remote or distance consultations or examinations with and for any individuals or patients, or caregivers, any data and/or information regarding the healthcare provider's name, address, telephone number, e-mail address, text messaging information or number(s), or any other contact information, credentials, education, practice area(s), insurance(s) accepted, fees, telephone number(s) or IP address(es) for video calls, video chat sessions, videoconferences, work schedule(s), appointment schedule(s), and/or any other information needed or desired for providing information regarding the healthcare provider to an individual, a patient, or a caregiver of or for the individual or the patient, and/or for allowing the individual, the patient, or the caregiver of the individual or the patient, to schedule a video call, a video chat session, or a videoconference, with the healthcare provider.

The database 110H can also contain, for each individual or patient who utilizes the apparatus 100 and method of the present invention, information regarding, or a record of, each and every instance of a provider office visit involving the individual or patient, a communication(s) with a provider or providers involving or regarding the individual or patient, a payment(s) made by or on behalf of the individual or patient, an insurance claim(s) made by a provider or providers on a healthcare insurance policy or account for or regarding the individual or patient, including information regarding date of claim, time of claim, or the provider making the claim, a co-payment(s) made by or on behalf of the individual or patient, a payment(s) made on an insurance claim, a denial(s) of an insurance claim or insurance claims, or a resolution(s) of an insurance claim or insurance claims. The database 110H can also contain information regarding a history or record of insurance claims made by a provider or providers, or made by an individual or patient, for each individual or patient who utilizes the apparatus 100 and method of the present invention.

The database 110H can also contain and/or include, for each individual, patient, or caregiver of or for the individual or the patient, any recordings of any of the herein-described video calls, video chat sessions, or video conferences, in which the individual, patient, or caregiver of or for the individual or the patient, was a party to or participated in.

The database 110H can also contain and/or include for each individual, patient, or caregiver of or for the individual or the patient, any of the herein-described video call reports, remote or virtual office visit reports, remote or distance examination reports, insurance claim forms, request for payment forms, and/or request for payment of co-payment forms, generated for, pertaining to, or associated with, the each individual, patient, or caregiver of or for the individual or the patient.

The database 110H can also contain credit report data or information, a credit report, credit reports, or a credit history, for any individual or patient, or for any of the individuals or patients, described herein or otherwise, who utilize the apparatus 100 and method of the present invention. The credit report or credit reports, or credit history, can contain any of the information which is typically provided by, or which may be provided by, any commercial or other credit reporting company or agency, such as, for example, but not limited to, credit reporting companies such as TransUnion LLC, Experian Information Solutions, Inc., Equifax, Inc., or any other credit reporting company, agency, or bureau, or any credit card company or financial institution. The database 110H can also contain any data or information which is typically found in a credit report database or credit history database, and/or any data and/or information which is, or which can be, utilized in generating, and/or analyzing, a credit report or credit reports, or a credit history, for, or on behalf of, any individual or patient, or for any of the individuals or patients, described herein or otherwise, who utilize the apparatus 100 and method of the present invention. The database 110H can also contain e-mail contact information, telephone contact information, cellular telephone contact information, and/or any other suitable contact information, for an individual or patient which can be utilized to notify the individual or patient when information regarding his or her credit report or credit reports, or credit history, is accessed, obtained, changed, or when a new entry of information is made in same.

The database 110H can also include advertisements, advertisement materials and information, marketing materials or information, commercials, video clips, infomercials, and any other information, which can include text information. video information, audio information, or any combination of same, which can be used to provide an advertisement, or advertisement material or information, to or for any individual, patient, provider, healthcare provider, payer, healthcare payer, insurer, healthcare insurer, product provider, service provider, third party provider, or any other person or entity, who or which utilizes the apparatus 100 and method of the present invention. A respective advertisement, advertisement material and information, marketing material or information, commercial, video clips, infomercials, and/or any other information, can be used to advertise or market any product(s) or service(s) which can be used by, provided to, provided by, advertised to, advertised by, marketed to, or marketed by, any individual, patient, provider, healthcare provider, payer, healthcare payer, insurer, healthcare insurer, product provider, service provider, third party provider, or any other person or entity, who or which utilizes the apparatus 100 and method of the present invention.

The database 110H can also contain news information, news reports, published reports, theses, study reports, or any other data or information regarding health issues, healthcare issues, health conditions, diseases, treatments, drugs, medications, healthcare procedures, medical procedures, surgical procedures, dental procedures, prescription products or services, non-prescription products or services, over-the-counter products or services, health or wellness issues or recommendations, exercise issues or recommendations, alternate therapy or treatment types or recommendations, herbal therapies or recommendations, or any other data or information which can be used by or provided to any individual, patient, provider, healthcare provider, payer, healthcare payer, insurer, healthcare insurer, product provider, service provider, third party provider, or any other person or entity, who or which utilizes the apparatus 100 and method of the present invention. The news information, news reports, published reports, theses, study reports, or any other data or information can include text information, video information, audio information, or any combination of same.

The news information, news reports, published reports, theses, study reports, or any other data or information, stored in the database 110H, can be transmitted to, or fed to, the central processing computer component 10A, for storage in the database 110H, by or from any appropriate computer or communication device, which is associated with or used by any news source, news organization, research institution, research professional, healthcare facility, an author, researcher, academician, or any other individual, person, or entity. An intermediary communication device 50 can also be utilized to transmit the news information, news reports, published reports, theses, study reports, or any other data or information to the central processing computer component 10A.

The database 110H can also contain prescription drugs information, prescription medication information, over-the-counter drug or medication information, wellness information, exercise and fitness information, and/or any other information pertinent to an individual's or a patient's heath, well-being, or treatment.

The database 110H can also include language translation information or software so that any of the data or information described herein as being stored in the database 110H, or as being provided in any of the messages, reports, or other communications, described herein can be translated into any language.

The database 110H can also contain, for each individual, patient, provider, and/or insurer or payer who or which utilize the apparatus 100, data and/or information regarding charges made to an individual or patient, charges made by a provider, charges made by an insurer or payer, amounts paid to an individual or patient, amounts paid by an individual or patient to a provider, amounts paid by an insurer or payer to a provider, amounts paid by an insurer or patient to an individual or patient, amounts paid by an individual or patient to an insurer or payer, a charges-back(s) made regarding a payment to, for, or by, a provider, a charge-back(s) made regarding a payment to, for, or by, an individual or patient, and/or a charge-back(s) made regarding a payment to, for, or by, an insurer.

The database 110H can also contain any notes, comments, or messages, which can be provided in text, in audio recordings or audio clips, and/or in video recordings or video clips. The database 110H can also contain any data, information, and/or software programs for translating audio information to text information and for translating test information to audio information. The database 110H can also contain any data, information, and/or software programs for translating text information or audio information from one language to another so as to provide a multi-lingual healthcare information and records keeping system. The database 110H can also contain any notes, comments, or messages, which can be provided by any of the herein-described users, individuals, patients, providers, insurers, payers, third parties, intermediaries, and/or any other person or entity who or which utilizes the apparatus 100 and method of the present invention.

The database 110H can also contain any notes, comments, or messages, which can be stored in any and/or all of the electronic healthcare records or electronic healthcare files, electronic medical records or files, electronic dental records or files, electronic pharmacy records or files, electronic behavioral health records or files, and/or personal health records or files, described herein as being utilized in connection with the apparatus 100 and method of the present invention. In this regard, it is important to note that, in a preferred embodiment of the present invention, any and/or all of the electronic healthcare records or electronic healthcare files, electronic medical records or files, electronic dental records or files, electronic pharmacy records or files, electronic behavioral health records or files, and/or personal health records or files, described herein as being utilized in connection with the apparatus 100 are or can be designed to receive, store, and/or provide, any notes, comments, and/or messages, provided by any of the herein-described users, individuals, patients, providers, insurers, payers, third parties, intermediaries, and/or any other person or entity, as text information or in text form, as audio information or in audio form, or as video information or in video form. Any text or audio information can be translated to from text to audio or from audio to text, and any text or audio information can be translated from one language to any one of more of any other language.

The database 110H can also contain sports medicine-related data and/of information for and including healthcare records, histories, or files, of and for athletes or sports participants of any age and in or for any sport or athletic activity. The database 110H can also contain data and/or information regarding healthcare records, histories, and/or files, for any athletes or sport participants and their respective organizations, teams, clubs, leagues, or other entities. The database 110H can also contain data and/or information for creating and for maintaining a comprehensive and/or centralized sports medicine healthcare record, history, and/or file, system for athletes or participants of any sport or sports or any activity or activities, athletes or sports participants of any age, professional athletes, minor league athletes, Olympic athletes, competitive athletes, government team athletes, world class competition athletes, amateur athletes, college athletes, high school age athletes, children athletes, secondary school athletes, recreational organization athletes or members, child recreational organization athletes or members, adult recreational organization athletes or members, little league athletes or members, boys club athletes or members, girls club athletes or members, hobbyist athletes, or any other individuals, male or female, who participate in athletic endeavors, sporting events, exercise activities or programs, the martial arts, mixed martial arts, boxing and other fighting or combat sports, any and/or all team sports, including but not limited to baseball, football, basketball, hockey, soccer, lacrosse, track and field, gymnastics, car racing, skiing and winter sports, swimming and aquatic sports, weightlifting, dancing, and/or any other sports or sporting activities, and/or other physical activities, or any team sporting activities or individual sporting activities.

The database 110H can also contain data and/or information regarding any organization, governmental entity, business, team, league, club, network, or any other entity, which oversees, manages, uses, employs, provides oversight over, owns or otherwise has playing or managing rights over, provides control over, or provides or is responsible for the healthcare of, any of the herein-described or other athletes or sports participants.

The database 110H can also contain data and/or information for or regarding sports-related injuries or athletic related injuries, or conditions, or other healthcare information relating to sports-related injuries, athletic injuries, sport-related conditions, and/or athletics-related conditions.

The database 110H can also contain data and/or information for diagnosing, treating, treatment planning, curing, rehabilitating, and/or for performing therapy or therapies for, sports-related injuries or conditions or athletic-related injuries or conditions. The database 110H can also contain information regarding diagnoses, treatments, treatment plans, procedures, corrective procedures or surgical procedures, cures, rehabilitation, therapies, physical therapies, exercise therapies, drug or medicinal therapies, diets, nutritional therapies, alternate medicine therapies, herbal therapies, and/or any other information which can or which may be utilized or needed for monitoring and/or managing the health and well-being of athletes or sports participants.

The database 110H can also contain data, information, or algorithms, for devising and planning treatments, treatment plans, procedures, operations, rehabilitation programs, plans, or regimens, physical therapy programs, plans, or regimens, occupational therapy programs, plans, or regimens, exercise programs, plans, or regimens, massage therapy programs, plans, or regimes, and/or any other programs, plans, or regimens for treating, curing, or otherwise dealing with, a sports-related injury or diagnosis.

The database 110H can also contain data and/or information regarding, for identifying or locating, healthcare providers, doctors, specialists, dentists, dental specialists, psychiatrists, psychologists, chiropractors, podiatrists, optometrists, or any other providers, hospitals, treatments facilities, therapists, nurses, physical therapists, massage therapists, occupational therapists, trainers, and/or any other providers or care givers, and/or testing laboratories, who or which can provide services for any other athletes and/or sports participants who may or which my utilize or be serviced by the apparatus 100 and method of the present invention, or any of their respective organizations, teams, clubs, or other entities.

The database 110H can also contain information, text information, audio, audio information or audio clips, video, video information, video clips, audio and video information, audio and video clips, news story, television programs, and/or information in any other form or type regarding fitness, fitness exercises, exercises, fitness routines, fitness regimens, wellness, wellness exercises, wellness routines, wellness regimens, diets, diet exercises, diet or dieting routines, diet ort dieting regimens, training, training principals, training exercises, training routines, training regimens, nutrition, health and well being, meditation and any other information.

The database 110H can also contain statistical and/or other probabilistic and/or mathematical information for assigning and/or correlating certain levels and/or estimates for any and/or all of the information, diagnoses, prognoses, treatments, procedures, and/or any other information processed and/or generated by the central processing computer component 10A and/or the apparatus 100.

The database 110H, in the preferred embodiment, can be a database which may include individual databases or collections of databases, with each database being designated to store any and all of the data and/or information described herein. The database 110H, or collection of databases, may be updated by each of the respective patients, providers, payers, users, and/or intermediaries, and/or by any other third party, in real-time, and/or via dynamically linked database management techniques.

The data and/or information stored in the database 110H can also be updated by and/or dynamically linked to, various external sources, including but not limited to news services, research publications, research facilities, healthcare laboratories, providers of healthcare goods and/or services, pharmaceutical companies, research institutions, schools. The database 110H will contain any and all information deemed necessary and/or desirable for providing all of the processing and/or services and/or functions described herein.

The database 110H can also contain data and/or information regarding any and/or all International Classification of Diseases (ICD) codes, ICD-10 codes, ICD-9, or any other ICD codes, or other codes, billing codes, diagnostic codes, treatment codes, or symptom codes, as well as any other codes or coding information which pertains to healthcare, the administration of healthcare services, healthcare record keeping, healthcare billing, healthcare diagnostics, healthcare treatments, or any other healthcare or healthcare-related functions or services, or any information or function, described-herein as capable of being provided by the apparatus 100 and method of the present invention. The database 110H can also contain data and/or information regarding the foreign language translations or conversions of or for, or for the foreign language translation or converting, of any of the herein-described International Classification of Diseases (ICD) codes, ICD-10 codes, ICD-9, any other ICD codes, or other codes, billing codes, diagnostic codes, treatment codes, symptom codes, as well as any other codes or coding information which pertains to healthcare, the administration of healthcare services, healthcare record keeping, healthcare billing, healthcare diagnostics, healthcare treatments, or any of the healthcare or healthcare-related functions or services, or any information or function described-herein as capable of being provided by the apparatus 100 and method of the present invention, into any one or any number of foreign languages.

The database 110H can contain any look-up tables, or information for use in a look table, for translating or converting any of the data, information, or codes, described herein, into any one or any number of foreign languages. The database 110H can also contain any language translation software or language conversion software which can be used to perform any of the foreign language translation or foreign language conversion processing or functionality described as being performed by the apparatus 100 and method of the present invention.

The database 110H can also contain any look-up tables, any master index tables, any data and/or information conversion information, and/or any other data and/or information, and/or software programs, software algorithms, software applications, which are needed or required for allowing the central processing computer component 10A to perform any healthcare records interoperability operations, functions, and/or processing routines, for updating all of the various healthcare records of or associated with any of the individuals and patients who utilize the apparatus 100 of the present invention. In this regard, the central processing computer component 10A can update all of an individual's or a patient's various and respective healthcare records of the various and respective healthcare providers of the individuals or patients, regardless of the manufacturer, source, or provider, of each respective healthcare record, upon the updating of any one healthcare record of the individual or patient. In this regard, the central processing computer component 10A can provide healthcare record interoperability for and of all the individual's or a patient's various and respective healthcare records.

The database 110H can also contain any information or software, or rules algorithms, which can be used in generating insurance claim forms which can be submitted error-free to any of the insurers or payers described herein.

The database 110H can also contain digital pictures, photographs, images, video, or video clips, of individuals or patients, as well as digital pictures, photographs, images, video, or video clips, of physical conditions of or regarding an individual or patient, which can be accessed or retrieved by any of the individuals, patients, providers, payers, or intermediaries, described herein as utilizing the apparatus 100 and method of the present invention. For example, in instances where photographs or images can be useful to see a state of a physical condition of a patient or individual, or to compare a state of a physical condition from one point in time to another, or to track the state of a physical condition, a photograph(s), picture(s), or image(s), or video clip, can be digitally recorded, with a camera or video recording device, and stored in the database 110H and/or in the patient's or individual's healthcare record or healthcare file for later retrieval, use, and/or review.

For example, a dermatologist seeking to determine if a skin condition has or is changing in size, shape, or color, can take and store, in the database 110H and/or in the patient's or individual's healthcare record or healthcare file, one or more photographs, pictures, or images, or a video clips, of the skin condition. In an analogous manner, a plastic surgeon can obtain and store, in the database 110H and/or in the patient's or individual's healthcare record or healthcare file, photographs, pictures, or images, or video clips, showing physical conditions of an individual or patient before, after, or during a procedure or procedures. A dentist or an oral health professional can obtain and store, in the database 110H and/or in the patient's or individual's healthcare record or healthcare file, in addition to x-rays, a photograph(s), picture(s), or image(s), or a video clip(s), showing physical conditions of an individual's or patient's mouth, teeth, gums, or other physical oral state, for later retrieval, use, or review. In an analogous manner, any healthcare provider can obtain and store, in the database 110H and/or in a patient's or individual's healthcare record or healthcare file, a photograph(s), picture(s), or image(s), or a video clip(s), obtained in or during an office visit, an examination, a procedure, an operation, a treatment, or during any other occasion, event, occurrence, or happening, for later retrieval and use as needed or desired by any provider, payer, patient, individual, or intermediary, described herein as being able to utilize the apparatus 100 and method of the present invention.

The database 110H can also contain video and/or audio clips or recordings of office visits, examinations, procedures, operations, or any other healthcare, healthcare-related, or any other event(s), occurrence(s), or happening(s), which may or which can be of interest to any provider, insurer, payer, patient, individual, or intermediary, described herein as being able to utilize the apparatus 100 and method of the present invention.

The database 110H can also contain transcripts of office visits, examinations, procedures, operations, or any other healthcare, healthcare-related, or any other event(s), occurrence(s), or happening(s), or any video calls, video chat sessions, or videoconferences of any a remote or virtual office visits or any remote or distance examinations, which may or which can be of interest to any provider, insurer, payer, patient, individual, or intermediary, described herein as being able to utilize the apparatus 100 and method of the present invention.

The database 110H can also contain data, information, links, or hyperlinks, which can allow a healthcare provider to create or generate and/or transmit prescriptions or electronic prescriptions, or prescription forms, to pharmacies or other providers, for a patient or individual, or prescriptions or electronic prescriptions for tests, analyses, analysis work-ups, procedures, therapy, physical therapy, or other healthcare or healthcare-related services, for a patient or individual, or which can allow a healthcare provider to transmit a referral or an electronic referral or referral form to other healthcare providers or other providers of goods or services for a patient or individual.

The database 110H can also contain data, information, links, or hyperlinks, which can allow a healthcare provider to perform remote control or monitoring of healthcare devices or equipment used in the treatment of, a care of, or a monitoring of, a patient or individual, while the patient or individual is at home, in a hospital, in a nursing home, in a care facility, or traveling in or on, respectively, a car, automobile, train, subway train, boat, helicopter, airplane, aircraft, or any other vehicle. The database 110H can also contain data, information, a link(s), or a hyperlink(s) which can allow a healthcare provided to remotely control healthcare devices or equipment, instruments, surgical instruments, and/or any other devices which can be used in performing a procedure, a surgery, or a surgical procedure, on an individual or patient, and/or to administer a treatment to the individual or patient, via data, information, link(s), or a hyperlink(s), contained in the individual's or patient's electronic healthcare record.

The database 110H can also contain data and/or information regarding artificial limbs, artificial organs, implanted or implantable devices, and/or prosthetic devices, and/or dental implants, dental braces, and/or any other devices associated with an individual or patient. The data and/or information regarding artificial limbs, artificial organs, implanted or implantable devices, and/or prosthetic devices, and/or dental implants, dental braces, and/or any other devices associated with an individual or patient can include manufacturer information, model information, date put into use or installed, if applicable, maintenance instructions, provider who or which prescribed, installed, implanted, or fitted same, dates examined or checked, and/or any other data or information regarding same.

The database 110H can also contain data, information, or software, for performing healthcare audits, for performing meaningful use information processing routines, for identifying or selecting individuals for clinical trials, experimental procedures, experimental treatments, healthcare focus groups, healthcare surveys, or other activities or events, or for performing any other processing routines which can advantageously use any of the information stored in the database 110H, any information stored in any of the herein-described healthcare records, files, or histories, described herein, or any other information, stored by the apparatus 100 of the present invention or any of its computers, communication devices, component parts, or devices, or any other information stored in, provided by, generated by, or used by, the apparatus 100 of the present invention. For example, with regards to clinical trials, experimental procedures, experimental treatments, healthcare focus groups, healthcare surveys, or other activities or events, the database 110H can contain information regarding whether or not an individual or patient can or may be a candidate for, be eligible to participate in, has agreed to be candidate for, or has agreed to participate in, clinical trials, experimental procedures, experimental treatments, healthcare focus groups, healthcare surveys, or other activities or events, or has agreed to be candidate for, or has agreed to participate in, a particular clinical trial(s), experimental procedure(s), experimental treatment(s), healthcare focus group(s), healthcare survey(s), or other activities or events.

The database 110H can also contain any data and/or information needed or used to determine or measure the effectiveness of, or side effects experienced or associated with, any medication(s), medicine(s), drug(s), dietary supplement(s), supplement(s), vitamin(s), nutrient(s), over-the-counter product(s) or substance(s), or the effectiveness of a procedure(s), treatment(s), treatment plan(s), care management plan(s), or any other good(s) or service(s) which can be offered in the marketplace.

The database 110H can also contain data and/or information relating to information regarding health conditions, healthcare conditions, illnesses, diagnoses, symptoms, treatments, treatment plans, care management plans, care management practices, insurance information, information regarding type of insurance, wellness information, healthcare practices, healthcare history patterns, healthcare trends, treatment trends, care management trends, statistics, statistical analyses, studies, study trends, or any other healthcare or healthcare-related information, for or regarding any number or type of groups of individuals or patients, and any data or information for determining same. The database 110H can also contain data or information regarding, or for determining, any of the above-described information for any number of groups of individuals or patients based on sex, gender, age, occupation, education, ethnicity, nationality, country of origin, religion, or any demographic or demographics, or any other criteria. The database 110H can also contain data or information regarding, or for determining statistical information or probability information regarding, any of the herein-described information.

The database 110H can also contain data or information for implementing and for performing drug formulary checks or analyses, as well as data or information for performing medication, medicine, or drug, reconciliation.

The database 110H can also contain immunization data or information.

The database 110H can also contain educational or instructional information, links or hyperlinks to same, information regarding instructions or procedures, or links or hyperlinks to same, in order to allow for the providing of patient-specific educational or instructional information or resources to patients or individuals, as well as to any of the herein-described healthcare providers, other providers, payers or insurers, or intermediaries or caregivers.

The database 110H can also contain data or information necessary or desired for administering employee benefits for any of the individuals or patients who utilize the apparatus 100 and method of the present invention.

The database 110H can also contain data or information necessary or desired for purchasing or enrolling in employee benefits for any of the individuals or patients who utilize the apparatus 100 and method of the present invention.

The database 110H can also contain data or information necessary or desired for allowing any individual or patient to make a request or a claim for, or pursuant to, or to file a request or a claim for, an employee benefit or employee benefits, for any of the individuals or patients who utilize the apparatus 100 and method of the present invention.

The database 110H can also contain data or information for facilitating, or for providing for, the portability of healthcare insurance or health insurance benefits, life insurance or life insurance benefits, or disability insurance or disability insurance benefits, or for providing for the portability of any other employee benefit or employee benefits.

The database 110H can also contain any of the messages or reports described herein as being generated by or provided by the apparatus 100 and method of the present invention, for later use by any provider, insurer, payer, patient, individual, or intermediary, or any other person or entity, described herein as being able to utilize the apparatus 100 and method of the present invention.

The database 110H can also contain any data, information, or software, for performing any of the processing routines or functionality described herein as being capable of being performed by, or provided by, the apparatus 100 of the present invention.

The data and/or information which is contained in and/or stored in the database 110H as well as any of the other databases 20H, 30H, 40H, and 50H, described herein can be obtained from the various patients, individuals, providers, payers, and/or intermediaries, who or which utilize and/or who or which are serviced by the present invention. For example, the respective patients, providers, payers, intermediaries, and/or other users, could fill out questionnaires, forms, narratives, claim forms, and/or any other information medium, in written form, electronically, and/or otherwise.

Data and/or information stored in the database 110H as well as any of the other databases described herein can be updated by multiple parties. For example, a patient may provide a medical history for his or her individual file, and his or her medical doctor can update the medical history information for the patient upon examining and/or treating him or her. The payer may also update the file with any associated payment or payment-related information. Should the patient go to another doctor or different type of doctor, all previous information would be available for, and can be updateable by, the next doctor.

The database 110H can also contain information regarding alternate medicine techniques, herbal techniques, meditation techniques, exercise techniques, self healing, faith healing, and/or other non-medicine treatments and/or techniques.

The database 110H can also include statistical data and/or information regarding diagnoses, and/or alternate diagnoses, treatment success, treatment failure, as well as statistical data and/or information regarding misdiagnoses. The database 110H also contains data and/or information regarding experimental treatments as well as statistical information regarding same, successes of same and failures of same.

The database 110H can also contain any other data and/or information, including software for performing any and all of the operations, routines, processing routines, and other functions and/or functionality described herein as being capable of being performed by the apparatus 100, the central processing computer(s) 10 and by any of the herein-described provider communication devices 20, insurer/payer communication devices 30, user communication/personal monitoring devices 40, intermediary communication devices 50, healthcare records computers 60, insurance exchange computers 70, social networking computers 80, and/or media computers 90.

The database 110H can also contain any and/or all of the herein-described information for providing electronic veterinary healthcare records or filed (EVHRs) which can be used for maintaining healthcare records or file for any types or kinds f animals, high value animals, exotic animals, pet of any kind or type, or any other types or kinds of animals. For example, electronic veterinary healthcare records or files can be maintained and used for race horses, circus animals, animals used in shows, service animals, animals used for breeding, household pets, dogs, cats, fish, birds, mammals of any kind or types, birds of any kinds or types, and/or any other animal for which an owner, caregiver, or other responsible person or entity, may find it desirable or necessary to keep a healthcare record or file. In a preferred embodiment, the database 110H can also contain, any of the data and/or information, or any analogous data and/or information, described herein as being stored or contained in the database 110H, for any animal for which an electronic veterinary healthcare record or file is maintained.

It is important to note that the apparatus 100 and method of the present invention can be utilized, in any and/or all of the embodiments described herein, in order to process and/or to provide veterinary healthcare information and/or veterinary healthcare-related information for and regarding any kind or type of animal or animals. In this regard, when used in veterinary applications, the terms “individual”, “patient”, “client”, “user” or the like, or their plural forms, also refer to any of the herein-described animals or pets or any other animals or pets.

The database 110H can also contain any data and/or information, as well as any links or hyperlinks to any of the herein-described data and/or information described herein as being stored in the respective databases 20H, 30H, 40H, of the respective provider communication devices 20, insurer/payer communication devices 30, an/or user communication/personal monitoring devices 40 or personal monitoring devices 40, as well as any data and/or information stored in any of the herein-described intermediary communication devices 50, healthcare records computers 60, insurance exchange computers 70, social networking computers 80, and/or media computers 90, described herein as being used in connection with the present invention, and/or the database 110H can contain data and/or information and/or links or hyperlinks to any data and/or information located or stored on, at, or in any of the provider communication devices 20, insurer/payer communication devices 30, user communication/personal monitoring devices 40, intermediary communication devices 50, healthcare records computers 60, insurance exchange computers 70, social networking computers 80, and/or media computers 90, described herein.

The database 110H can also contain information, links and/or hyperlinks, to any social networking web sites, web pages, support groups, on-line forums, on-link information services, as well as social networking web sites or social networking web pages to or for social networking members, support groups, information providers, healthcare providers, as well as any of the providers, insurers, payers, individuals, patient, third parties, intermediaries, or any other persons or entities described herein who are or who may be members of any social network.

The database 110H can also contain data and/or information regarding any animals or pets associated with any individual, patient, or caregiver as well as any data and/or information regarding any electronic healthcare record for respective animal or pet.

The database 110H can also contain, for each or any of the herein-described individuals, patients, or caregiver, or for each provider or payer or insurer of a respective individual, patient, or caregiver, financial information regarding insurance premiums or payments, or premiums or payments for an insurer or payer policy, plan or program, which are or were made by or on behalf of the individual, patient, or caregiver, any and/or any insurance claim(s) or request(s) for payment(s) made by or on behalf of the individual, patient, or caregiver, provider bills or provider charges submitted in an insurance claim or a request for payment, provider amounts or provider charges billed for goods, products, or services, amounts paid by an insurer or payer for the amounts billed or charged by a provider, amounts paid or amounts of co-payments paid by or on behalf of the individual, patient, or caregiver, for the amounts billed, deductibles paid by or on behalf of the individual, patient or caregiver, and/or any other data and/or information regarding charges, bills, insurance claims, requests for payments, amounts paid by insurers or payers, and/or any amounts paid by or on behalf of the respective individual, patient, or caregiver.

The database 110H can also contain, for each individual, patient, or caregiver, any data and/or information regarding any vehicle(s) owned or operated by the individual, patient, or caregiver, along with data and/or information, and/or a link(s) or hyperlink(s) to any manufacturer of the vehicle or to any telematics, on-line service provider, or other services provider for the vehicle, such as for example, a telematics, on-line services provider, or other services provider who or which provides products and services such as those offered and provided in connection with automobiles and other motor vehicles as of the filing data of this application.

In any and/or all of the embodiments described herein, any of the data and/or information which is or which may be stored in the database 110H, and/or any of the other databases described herein, can be utilized and/or can appear in any of the reports, diagnostic reports, treatment reports, evaluation reports, provider reports, payer reports, patient reports, training reports, and/or any other reports, described herein.

In a preferred embodiment, the database 110H can also store a user profile for each user, individual, patient, caregiver, and/or healthcare provider, healthcare professional, hospital, clinic, pharmacy, treatment center, treatment facility, and/or any other provider of services described herein, insurer, payer, insurer employee or agent, payer employee or agent, intermediary, governmental entity employee or agent, who or which utilizes the apparatus 100 of the present invention. In a preferred embodiment, the user profile can contain or include a name, address, email address, telephone number, text message number, cellular telephone number, file photograph, educational information, training information, work experience information, and./or any other information regarding the respective user, individual, patient, caregiver, and/or healthcare provider, healthcare professional, hospital, clinic, pharmacy, treatment center, treatment facility, and/or any other provider of services described herein, insurer, payer, insurer employee or agent, payer employee or agent, intermediary, governmental entity employee or agent, who or which utilizes the apparatus 100 of the present invention.

In a preferred embodiment, database 110H can also contain and/or include video recordings of any and/or all remote healthcare visits, remote doctor visits, and/or telehealth visits, for and/or regarding each user, individual, or patient.

In a preferred embodiment, the database 110H can also contain and/or include educational videos for providing instruction to users, individuals, patients, caregivers, healthcare providers, insurers, payers, governmental entity employees and agents, and intermediaries, as well any other individuals or entities.

In a preferred embodiment, the database 110H can also contain any non-confidential data and/or information which is described herein as being stored or contained in the respective databases of the respective provider communication devices 20, insurer/payer communication devices 30, user communication/personal monitoring devices 40, intermediary communication devices 50, healthcare records computers 60, insurance exchange computers 70, social networking computers 80, and/or media computers 90.

The central processing computer component 10A also includes an output device 1101 for output any data, information, report, etc., described herein. In the preferred embodiment, the output device 1101 can be a printer, a display, a transmitter, a modem, a speaker, and/or any other device which can be used to output data.

The central processing computer component 10A can also include a video and/or audio recording device(s) 110J which can include a camera and/or a video recording device for recording pictures or video and/or video clips and/or a microphone or an audio recording device for recording audio or audio clips which can be recorded by, and/or transmitted live, by or from the central processing computer component 10A, or which can be recorded by, and stored at or in, the central processing computer component 10A for transmission by or from the central processing computer component 10A at a later time. The video and/or audio recording device(s) 110J can be utilized to facilitate video conferencing, video chatting, and/or audio conferencing, and/or video and audio conferencing, between users of the central processing computer component 10A and any user of any other computer or communication device 10, 20, 30, 50, 60, 80, and/or 90, described herein. In the preferred embodiment, the video and/or audio recording device(s) 110J is/are also connected to, or linked with, the CPU 110A.

With reference to FIG. 2, the central processing computer component 10A can also include a global positioning system (GPS) device 110K which can be utilized to determine or ascertain the location or position of the central processing computer component 10A at any time and/or to track movement of the central processing computer component 10A if the same is associated with a mobile platform. In a preferred embodiment, the global positioning system (GPS) device 110K can be connected to, or linked with, the CPU 110A.

Any of the data and/or information for any of the patients, individuals, providers, payers, and/or intermediaries, can be updated by different parities and which such updated data and/or information being made available to other respective parties so as to provide and ensure comprehensive and up-to-date healthcare and healthcare-related information.

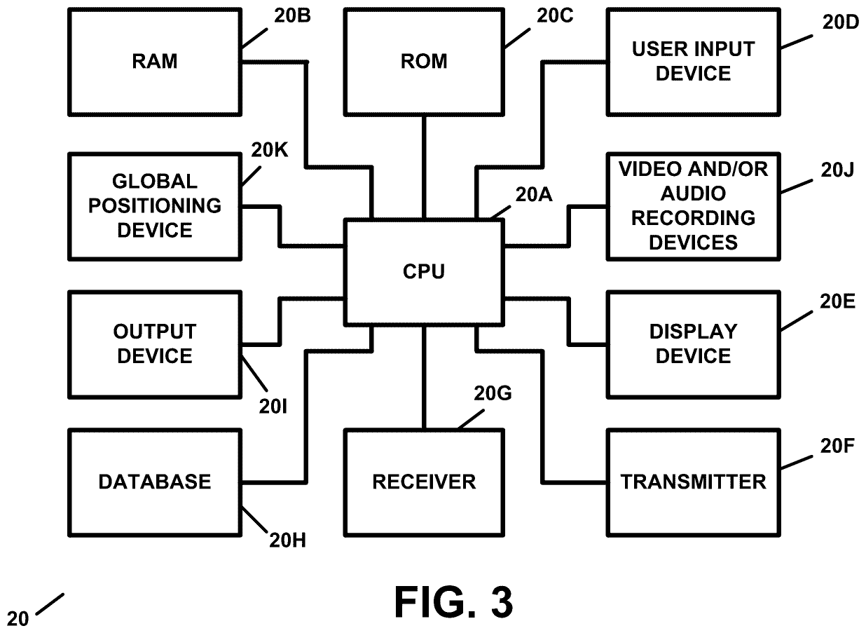

FIG. 3 illustrates the provider communication device 20 of FIG. 1, in block diagram form. The provider communication device 20, in the preferred embodiment, can be personal computer, a network computer or computer system, a cellular telephone, a wireless telephone, a smart phone or Smartphone, a watch, or a smart watch, or any other computer or communication device. In the preferred embodiment, the provider communication device 20 includes a central processing unit or CPU 20A, which in the preferred embodiment, is a microprocessor. The CPU 20A may also be a microcomputer, a minicomputer, a macro-computer, and/or a mainframe computer, depending upon the application.

The provider communication device 20 also includes a random access memory device(s) 20B (RAM) and a read only memory device(s) 20C (ROM), each of which is connected to the CPU 20A, a user input device 20D, for entering data and/or commands into the provider communication device 20, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information, which can also include, for example, pulse rate monitors, blood pressure monitors, electrocardiograms, glucose monitors, blood-sugars monitors, etc., if desired, or any other devices, measuring or measurement devices or equipment, or any other equipment used for obtaining information regarding an individual or patient, which input device(s) are also connected to the CPU 20A. In another preferred embodiment, the pulse rate monitors, blood pressure monitors, electrocardiograms, glucose monitors, blood-sugars monitors, etc., if desired, or any other devices, measuring or measurement devices or equipment, or any other equipment used for obtaining information regarding an individual or patient, can also be integrated into the provider communication device 20.

The user input device 20D can also acquire, receive, generate and/or provide, data which can be entered by a user or individual and/or can be a device which can acquire, receive, generate and/or provide, digital data such as medical, healthcare, bio-metric, physiological, and/or any other kind of healthcare data and/or healthcare-related data. For example, a user input device 20D can be a keyboard for allowing a user to input information into the provider communication device and/or a user input device can, for example, by a heart rate monitor or EKG machine which can receive information from a patient or individual and generate a digital signal, digital data, analog data, and/or any other signal, data, and/or information, which is representative of the patient's or individual's heart rate, pulse rate, and/or cardiac activity.

The user input device 20D can also be, or can include, any one or more of, and/or any combination of, a thermometer, a digital thermometer, a stethoscope, a heart rate monitor or measurement device, a pulse rate monitor or measurement device, a blood pressure monitor or measurement device, a blood oxygen or percentage oxygen measurement device, a blood pressure measurement device, blood analysis device or machine, a respirator, a respiration monitoring or measurement device, a dialysis machine, a dialysis device, electrocardiograph (EKG) machine or device, electrocephalograph (EEG) machine or device, electromyograph (EMG) machine or device, a magnetic resonance imaging (MRI) machine or device, X-ray machine or device, medical imaging machine or device, thermal imaging machine or device, a heart sound monitor or measurement device, a lung sound monitoring or measurement device, respiration rate monitoring or measurement device, a laparascopic device, an arthroscopic device, a vascular testing device, a catheter device, a cardiac performance testing, monitoring, or measurement device, a pulmonary performance testing, monitoring, or measurement device, a vascular system performance monitoring or measurement device, a vascular system testing, monitoring, or measurement device, a metabolism monitoring or measurement device, a sonogram imaging device, a sonogram measurement device, a sonograph device, an optical response device, an optical response measurement device, an intravenous device, an arterial blood pressure measurement or monitoring device, a respiration rate measurement or monitoring device, an ultrasound imaging device, an ultrasound measurement device, a CAT SCAN device, a PET scan device, an optical metabolism measurement or monitoring device, a radiotelemetric device, a doppler medical device, a mammogram device, a carbon dioxide detection or measurement device, a carbon monoxide detection or measurement device, a transvascular impedance measurement or monitoring device, an ultrasonic imaging device, a bone conduction device, a brain function scan analyzer device, external pulse cardiac monitoring or measurement device, a fetal heart rate measurement, monitoring, or probing, device, an endotracheal cardiac monitoring device, a finger tip blood pressure monitoring device, a psychological monitoring device, a surgical instrument, a vital signs measurement device, ear pressure regulating device, phonocardiograph device, acoustic aneurysm detector device, blood oxygen detection device, esophageal probing device, ultrasonic probing device, ausculoscope, vital signs monitoring system, heart activity monitoring device, pulmonary activity monitoring device, sphygmomanometer, esophageal stethoscope, venous pressure measuring device, differential doppler device, physiological data measuring device, body tissue movement device, breathalyzer device, camera probing device, microscopic camera probing device, any scope having a camera and/or a light, a laparoscopic device, a laparoscopic camera device, a camera used in or associated with equipment for performing visual probing of the body or any portions, parts, or organs, thereof, such as, but not limited to, a colonoscopy, an endoscopy, an esophagoscopy, a gastroscopy, or any devices used in laparoscopic surgery or other surgeries, an ingestible or implantable pill, capsule, or device, containing a camera and/or a light and equipment or circuitry for transmitting information as well as images to equipment inside or outside the body, and/or any other bio-metric or physiological data measuring device(s) and/or data acquisition device (hereinafter referred to generally or collectively as “healthcare equipment input device”, “healthcare measurement input device”, or “healthcare monitoring input device”).

The user input device 20D can also be, or can include, a pacemaker, a device for monitoring blood pressure or blood flow, or an ingestible or implantable pill, capsule, or device, containing a camera and/or a light and equipment or circuitry for transmitting information as well as images to equipment inside or outside the body.

The user input device 20D can also be, or can include, a global positioning device which can be utilized for determining a position or location of the provider communication device 20 and/or the provider who or which is using the same.

The provider communication device 20 also includes a display device 20E for displaying data and/or information to a user or operator.

The provider communication device 20 also includes a transmitter(s) 20F, for transmitting signals and/or data and/or information to any one or more of the central processing computer/distributed ledger/Blockchain technology system 10, to any other provider communication device(s) 20, and/or to any one or more of the insurer/payer communication devices 30, user communication/personal monitoring devices 40, intermediary communication devices 50, healthcare records computers 60, insurance exchange computers 70, social networking computers 80, and/or media computers 90, which may be utilized in conjunction with the present invention. The provider communication device 20 also includes a receiver 20G, for receiving signals and/or data and/or information from any one or more of the central processing computer/distributed ledger/Blockchain technology system 10, from any other provider communication device(s) 20, and/or from any one or more of the insurer/payer communication devices 30, user communication/personal monitoring devices 40, intermediary communication devices 50, healthcare records computers 60, insurance exchange computers 70, social networking computers 80, and/or media computers 90, which may be utilized in conjunction with the present invention.

The provider communication device 20 also includes a database(s) 20H. The database 20H can contain and/or be linked to any of the data and/or information described herein as being stored in the database 110H.

In a preferred embodiment, the database 20H of the provider communication device 20 also contains any and/or all artificial intelligence (AI) software programs, software algorithms, software applications, machine language software programs, software algorithms, software applications, and/or natural language software programs, software algorithms, software applications, which are needed to required for performing any and/or all of the functions, functionalities, processing routines, and/or operations described herein as being performed by the provider communication device 20.

In a preferred embodiment, the database 20H also contains any and/or all of the data, information, software programs, software algorithms, software applications, which is/are described as being stored in any the respective databases in, of, or associated with, any of the various and respective provider communication devices which are disclosed and described in each of U.S. patent application Ser. No. 16/202,448, U.S. patent application Ser. No. 14/638,934, U.S. Provisional Patent Application Ser. No. 61/971,225, U.S. patent application Ser. No. 17/224,838, U.S. patent application Ser. No. 18/233,321, U.S. patent application Ser. No. 18/225,734, U.S. patent application Ser. No. 18/223,700, U.S. Pat. Nos. 7,464,040, 7,490,048, and 10,571,284, the subject matter and teachings of all of which are herein incorporated by reference in their entirety as if to be fully restated herein.

The database 20H can also contain any look-up tables, any master index tables, any data and/or information conversion information, and/or any other data and/or information, and/or software programs, software algorithms, software applications, which are needed or required for allowing the provider communication device 20 to perform any healthcare records interoperability operations, functions, and/or processing routines, for updating all of the various healthcare records of or associated with any of the individuals and patients who utilize the apparatus 100 of the present invention. In this regard, the provider communication device 20 can update all of an individual's or a patient's various and respective healthcare records of the various and respective healthcare providers of the individuals or patients, regardless of the manufacturer, source, or provider, of each respective healthcare record, upon the updating of any one healthcare record of the individual or patient. In this regard, the provider communication device 20 can provide healthcare record interoperability for and of all the individual's or a patient's various and respective healthcare records.

The provider communication device 20 also includes an output device 201 for output any data, information, report, etc., described herein. In the preferred embodiment, the output device 201 can be a printer, a display, a transmitter, a modem, a speaker, and/or any other device which can be used to output data.

The provider communication device 20 also includes a video and/or audio recording device(s) 20J which can include a camera and/or a video recording device for recording pictures or video and/or video clips and/or a microphone or an audio recording device for recording audio or audio clips which can be stored in any of the electronic healthcare, electronic medical, electronic dental, electronic pharmacy, and/or electronic behavioral healthcare, records or files described herein, and/or which can be otherwise used for communicating with other users, individuals, patients, providers, payers, or persons or entities who or which utilize the apparatus 100 and method of the present invention.

With reference to FIG. 3, the provider communication device 20 also includes a global positioning device 20K. In a preferred embodiment, the global positioning device 20K determines a position or location of the provider communication device 20.

In the preferred embodiment, each of the RAM 20B, the ROM 20C, the user input device 20D, the display device 20E, the transmitter 20F, the receiver 20G, the database 20H, the output device 201, the video and/or audio recording devices 20J, and the global positioning device 20K are connected with, or linked to, the CPU 20A.

In a preferred embodiment, the provider communication device 20 can be equipped and/or can be programmed for voice activation operation and/or voice control operation of and/or regarding any and/or all of the herein-described operations and functions which are described herein and capable of being performed by the provider communication device 20.

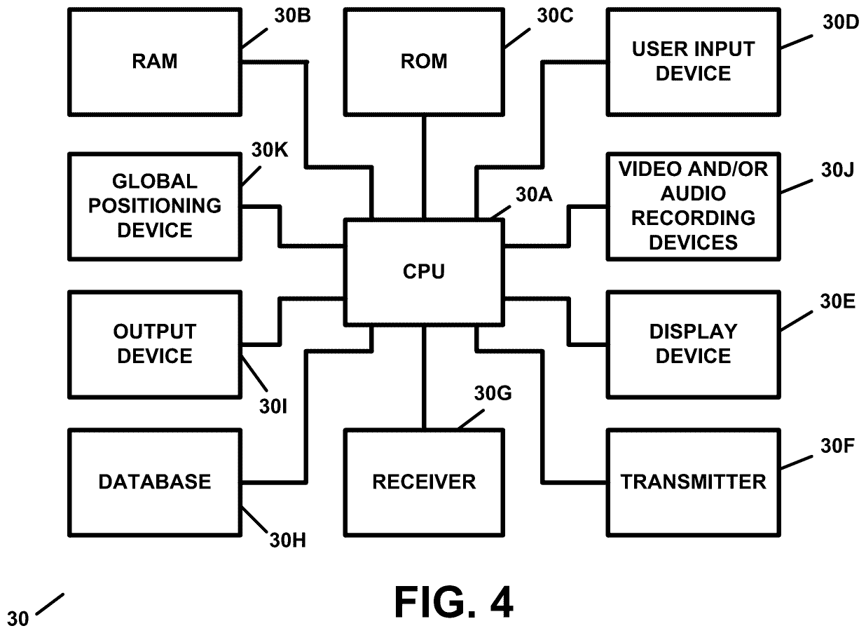

FIG. 4 illustrates the insurer/payer communication device 30 of FIG. 1, in block diagram form. The insurer/payer communication device 30, in the preferred embodiment, can be personal computer, a network computer or computer system, a cellular telephone, a wireless telephone, a smart phone or Smartphone, a watch, or a smart watch, or any other computer or communication device. In the preferred embodiment, the insurer/payer communication device 30 includes a central processing unit or CPU 30A, which in the preferred embodiment, is a microprocessor. The CPU 30A may also be a microcomputer, a minicomputer, a macro-computer, and/or a mainframe computer, depending upon the application.

The insurer/payer communication device 30 also includes a random access memory device(s) 30B (RAM) and a read only memory device(s) 30C (ROM), each of which is connected to the CPU 30A, a user input device 30D, for entering data and/or commands into the insurer/payer communication device 30, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information, which can also include, for example, pulse rate monitors, blood pressure monitors, electrocardiograms, glucose monitors, blood-sugars monitors, etc., if desired, or any other devices, measuring or measurement devices or equipment, or any other equipment used for obtaining information regarding an individual or patient, which input device(s) are also connected to the CPU 30A. In another preferred embodiment, the pulse rate monitors, blood pressure monitors, electrocardiograms, glucose monitors, blood-sugars monitors, etc., if desired, or any other devices, measuring or measurement devices or equipment, or any other equipment used for obtaining information regarding an individual or patient, can also be integrated into the insurer/payer communication device 30.

The user input device 30D can also acquire, receive, generate and/or provide, data which can be entered by a user or individual and/or can be a device which can acquire, receive, generate and/or provide, digital data such as medical, healthcare, bio-metric, physiological, and/or any other kind of healthcare data and/or healthcare-related data. For example, a user input device 30D can be a keyboard for allowing a user to input information into the provider communication device and/or a user input device can, for example, by a heart rate monitor or EKG machine which can receive information from a patient or individual and generate a digital signal, digital data, analog data, and/or any other signal, data, and/or information, which is representative of the patient's or individual's heart rate, pulse rate, and/or cardiac activity.

The user input device 30D can also be, or can include, any one or more of, and/or any combination of, a thermometer, a digital thermometer, a stethoscope, a heart rate monitor or measurement device, a pulse rate monitor or measurement device, a blood pressure monitor or measurement device, a blood oxygen or percentage oxygen measurement device, a blood pressure measurement device, blood analysis device or machine, a respirator, a respiration monitoring or measurement device, a dialysis machine, a dialysis device, electrocardiograph (EKG) machine or device, electrocephalograph (EEG) machine or device, electromyograph (EMG) machine or device, a magnetic resonance imaging (MRI) machine or device, X-ray machine or device, medical imaging machine or device, thermal imaging machine or device, a heart sound monitor or measurement device, a lung sound monitoring or measurement device, respiration rate monitoring or measurement device, a laparascopic device, an arthroscopic device, a vascular testing device, a catheter device, a cardiac performance testing, monitoring, or measurement device, a pulmonary performance testing, monitoring, or measurement device, a vascular system performance monitoring or measurement device, a vascular system testing, monitoring, or measurement device, a metabolism monitoring or measurement device, a sonogram imaging device, a sonogram measurement device, a sonograph device, an optical response device, an optical response measurement device, an intravenous device, an arterial blood pressure measurement or monitoring device, a respiration rate measurement or monitoring device, an ultrasound imaging device, an ultrasound measurement device, a CAT SCAN device, a PET scan device, an optical metabolism measurement or monitoring device, a radiotelemetric device, a doppler medical device, a mammogram device, a carbon dioxide detection or measurement device, a carbon monoxide detection or measurement device, a transvascular impedance measurement or monitoring device, an ultrasonic imaging device, a bone conduction device, a brain function scan analyzer device, external pulse cardiac monitoring or measurement device, a fetal heart rate measurement, monitoring, or probing, device, an endotracheal cardiac monitoring device, a finger tip blood pressure monitoring device, a psychological monitoring device, a surgical instrument, a vital signs measurement device, ear pressure regulating device, phonocardiograph device, acoustic aneurysm detector device, blood oxygen detection device, esophageal probing device, ultrasonic probing device, ausculoscope, vital signs monitoring system, heart activity monitoring device, pulmonary activity monitoring device, sphygmomanometer, esophageal stethoscope, venous pressure measuring device, differential doppler device, physiological data measuring device, body tissue movement device, breathalyzer device, camera probing device, microscopic camera probing device, any scope having a camera and/or a light, a laparoscopic device, a laparoscopic camera device, a camera used in or associated with equipment for performing visual probing of the body or any portions, parts, or organs, thereof, such as, but not limited to, a colonoscopy, an endoscopy, an esophagoscopy, a gastroscopy, or any devices used in laparoscopic surgery or other surgeries, an ingestible or implantable pill, capsule, or device, containing a camera and/or a light and equipment or circuitry for transmitting information as well as images to equipment inside or outside the body, and/or any other bio-metric or physiological data measuring device(s) and/or data acquisition device (hereinafter referred to generally or collectively as “healthcare equipment input device”, “healthcare measurement input device”, or “healthcare monitoring input device”).

The user input device 30D can also be, or can include, a pacemaker, a device for monitoring blood pressure or blood flow, or an ingestible or implantable pill, capsule, or device, containing a camera and/or a light and equipment or circuitry for transmitting information as well as images to equipment inside or outside the body.

The user input device 30D can also be, or can include, a global positioning device which can be utilized for determining a position or location of the insurer/payer communication device 30 and/or the provider who or which is using the same.

The insurer/payer communication device 30 also includes a display device 30E for displaying data and/or information to a user or operator.

The insurer/payer communication device 30 also includes a transmitter(s) 30F, for transmitting signals and/or data and/or information to any one or more of the central processing computer/distributed ledger/Blockchain technology system 10, to any other insurer/payer communication device 30, and/or to any one or more of the provider communication device(s) 20, user communication/personal monitoring devices 40, intermediary communication devices 50, healthcare records computers 60, insurance exchange computers 70, social networking computers 80, and/or media computers 90, which may be utilized in conjunction with the present invention. The insurer/payer communication device 30 also includes a receiver 30G, for receiving signals and/or data and/or information from any one or more of the central processing computer/distributed ledger/Blockchain technology system 10, from any other insurer/payer communication device 30, and/or from any one or more of the provider communication device(s) 20, user communication/personal monitoring devices 40, intermediary communication devices 50, healthcare records computers 60, insurance exchange computers 70, social networking computers 80, and/or media computers 90, which may be utilized in conjunction with the present invention.

The insurer/payer communication device 30 also includes a database(s) 30H. The database 30H can contain and/or be linked to any of the data and/or information described herein as being stored in the database 110H.

In a preferred embodiment, the database 30H of the insurer/payer communication device 30 also contains any and/or all artificial intelligence (AI) software programs, software algorithms, software applications, machine language software programs, software algorithms, software applications, and/or natural language software programs, software algorithms, software applications, which are needed to required for performing any and/or all of the functions, functionalities, processing routines, and/or operations described herein as being performed by the insurer/payer communication device 30.

In a preferred embodiment, the database 30H also contains any and/or all of the data, information, software programs, software algorithms, software applications, which is/are described as being stored in any the respective databases in, of, or associated with, any of the various and respective insurer/payer communication device which are disclosed and described in each of U.S. patent application Ser. No. 16/202,448, U.S. patent application Ser. No. 14/638,934, U.S. Provisional Patent Application Ser. No. 61/971,225, U.S. patent application Ser. No. 17/224,838, U.S. patent application Ser. No. 18/233,321, U.S. patent application Ser. No. 18/225,734, U.S. patent application Ser. No. 18/223,700, U.S. Pat. Nos. 7,464,040, 7,490,048, and 10,571,284, the subject matter and teachings of all of which are herein incorporated by reference in their entirety as if to be fully restated herein.

The database 30H can also contain any look-up tables, any master index tables, any data and/or information conversion information, and/or any other data and/or information, and/or software programs, software algorithms, software applications, which are needed or required for allowing the insurer/payer communication device 30 to perform any healthcare records interoperability operations, functions, and/or processing routines, for updating all of the various healthcare records of or associated with any of the individuals and patients who utilize the apparatus 100 of the present invention. In this regard, the insurer/payer communication device 30 can update all of an individual's or a patient's various and respective healthcare records of the various and respective healthcare providers of the individuals or patients, regardless of the manufacturer, source, or provider, of each respective healthcare record, upon the updating of any one healthcare record of the individual or patient. In this regard, the insurer/payer communication device 30 can provide healthcare record interoperability for and of all the individual's or a patient's various and respective healthcare records.

The insurer/payer communication device 30 also includes an output device 301 for output any data, information, report, etc., described herein. In the preferred embodiment, the output device 301 can be a printer, a display, a transmitter, a modem, a speaker, and/or any other device which can be used to output data.

The insurer/payer communication device 30 also includes a video and/or audio recording device(s) 30J which can include a camera and/or a video recording device for recording pictures or video and/or video clips and/or a microphone or an audio recording device for recording audio or audio clips which can be stored in any of the electronic healthcare, electronic medical, electronic dental, electronic pharmacy, and/or electronic behavioral healthcare, records or files described herein, and/or which can be otherwise used for communicating with other users, individuals, patients, providers, payers, or persons or entities who or which utilize the apparatus 100 and method of the present invention.

With reference to FIG. 4, the insurer/payer communication device 30 also includes a global positioning device 30K. In a preferred embodiment, the global positioning device 30K determines a position or location of the insurer/payer communication device 30.

In the preferred embodiment, each of the RAM 30B, the ROM 30C, the user input device 30D, the display device 30E, the transmitter 30F, the receiver 30G, the database 30H, the output device 301, the video and/or audio recording devices 30J, and the global positioning device 30K are connected with, or linked to, the CPU 30A.

In a preferred embodiment, the insurer/payer communication device 30 can be equipped and/or can be programmed for voice activation operation and/or voice control operation of and/or regarding any and/or all of the herein-described operations and functions which are described herein and capable of being performed by the insurer/payer communication device 30.

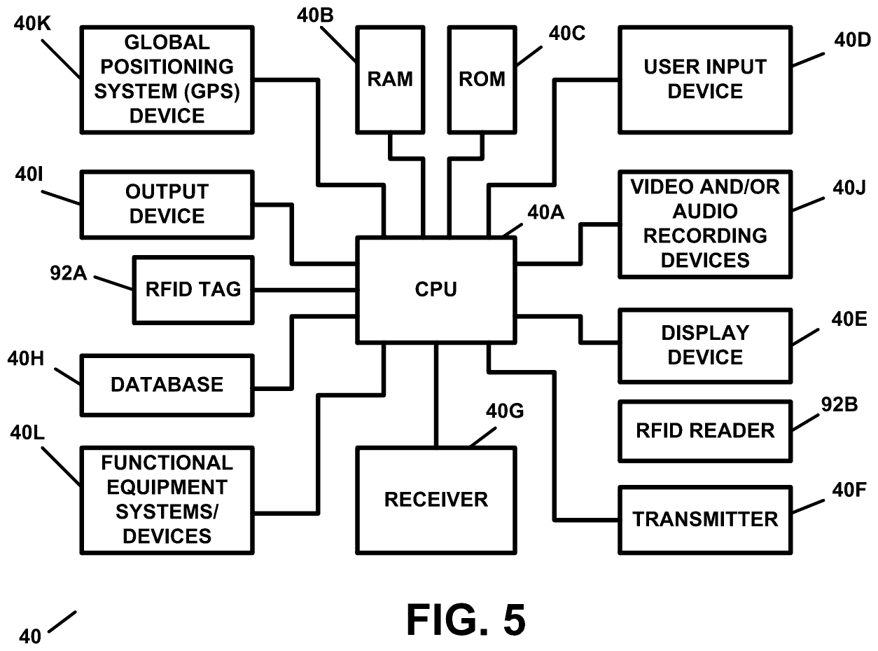

FIG. 5 illustrates the user communication/personal monitoring devices 40 of FIG. 1, in block diagram form. In a preferred embodiment, the user communication/personal monitoring device 40 can be a cellular telephone, a wireless telephone, a mobile telephone, a Smartphone, a smartphone, or a personal digital assistant, or the user communication/personal monitoring device 40 can be or can be a component of a personal computer, a home computer, a laptop computer, a notebook computer, a tablet computer, a tablet, a hand-held computer, a palmtop computer, a personal communication device, a cellular telephone, a wireless telephone, a mobile telephone, a Smartphone, a smartphone, a personal digital assistant, a digital television, an interactive television, a digital television, a telephone, a digital telephone, a television, an interactive television, a beeper, a pager, and/or a watch or a Smart watch, and/or any wearable device, computer or communication device. The user communication/personal monitoring device 40 can also be a server computer, or any computer capable of being utilized in a network or capable of being utilized with other computers in a network.

The user communication/personal monitoring device 40 can also be any communication device, computer, personal computer, laptop computer, notebook computer, Smartphone or smartphone, smart telephone, cellular telephone, personal digital assistant, tablet, tablet computer, watch, smart watch, or wearable device or computer, an implantable device or computer, an item of jewelry, eyeglasses, or any accessory, or any combination of same, or any equivalent of same.

With reference to FIG. 5, in the preferred embodiment, the user communication/personal monitoring device 40 includes a central processing unit or CPU 40A, which in the preferred embodiment, is a microprocessor. The CPU 40A can also be a microcomputer, a minicomputer, a macro-computer, and/or a mainframe computer, depending upon the application.

The user communication/personal monitoring device 40 also includes a random access memory device(s) 40B (RAM), a read only memory device(s) 40C (ROM), each of which is connected to, or linked with, the CPU 40A, and a user input device 40D, for inputting and/or entering data and/or information and/or instructions and/or commands into the user communication/personal monitoring device 40, which can include any one or more of a keyboard, a scanner, a user pointing device, such as, for example, a mouse, a stylus, a touch pad, and/or an audio input device, a microphone, an audio recording device, and/or a video input device, a camera or any number of cameras, a video recording device, and/or any device, electronic and/or otherwise, which can be utilized for inputting and/or entering data and/or information and/or instructions and/or commands into the user communication/personal monitoring device 40.

The input device(s) 40D is/are also connected to, or linked with, the CPU 40A. In a preferred embodiment, the input device(s) 40D can also be or can include a pulse rate monitor or measurement device or equipment, a heart rate monitor of measurement device or equipment, a blood sugar monitor or measurement device or equipment, a glucose monitor or measurement device or equipment, a blood pressure monitor or measurement device or equipment, a blood alcohol monitor or measurement device or equipment, a pacemaker, a defibrillator, a thermometer for measuring an individual's body temperature, a pulse oximeter for measuring and/or monitoring oxygen saturation and/or pulse rate, an oximeter, a digital finger pulse oximeter, an electrocardiogram monitor or device, an EKG monitor or device, an electroencephalogram monitor or device, an EEG monitor or device, an electromyography monitor or device, an EMG monitor or device, a defibrillator, a pacemaker, or any other device, monitor, or measurement, device or equipment, and/or any electrical or bio-medical device or equipment, which can measure an individual's physical condition, health condition, health status, healthcare condition, or physiological condition or status, or any other biological or biometric data and/or information. In a preferred embodiment, any pulse rate monitor or measurement device or equipment, heart rate monitor of measurement device or equipment, blood sugar monitor or measurement device or equipment, glucose monitor or measurement device or equipment, blood pressure monitor or measurement device or equipment, blood alcohol monitor or measurement device or equipment, pacemaker, defibrillator, thermometer for measuring an individual's body temperature, pulse oximeter for measuring and/or monitoring oxygen saturation and/or pulse rate, oximeter, digital finger pulse oximeter, electrocardiogram monitor or device, EKG monitor or device, electroencephalogram monitor or device, EEG monitor or device, electromyography monitor or device, EMG monitor or device, defibrillator, pacemaker, or any other device, monitor, or measurement, device or equipment, and/or any electrical or bio-medical device or equipment, which can measure an individual's physical condition, health condition, health status, healthcare condition, or physiological condition or status, or any other biological or biometric data and/or information, can be an input device 40D, or can be a component of any input device 40D, can be connected via a wired connection to the user communication/personal monitoring device 40, can be connected via a wireless connection to the user communication/personal monitoring device 40D, and/or can be connected via a Bluetooth or other suitable or appropriate wireless technology.

The user input device 40D can also be, or can include, any one or more of, and/or any combination of, a thermometer, a digital thermometer, a stethoscope, a heart rate monitor or measurement device, a pulse rate monitor or measurement device, a blood pressure monitor or measurement device, a blood oxygen or percentage oxygen measurement device, a blood pressure measurement device, a blood analysis device or machine, a respirator, a respiration monitoring or measurement device, a dialysis machine, a dialysis device, electrocardiograph (EKG) machine or device, electrocephalograph (EEG) machine or device, electromyograph (EMG) machine or device, a magnetic resonance imaging (MRI) machine or device, X-ray machine or device, medical imaging machine or device, thermal imaging machine or device, a heart sound monitor or measurement device, a lung sound monitoring or measurement device, respiration rate monitoring or measurement device, a laparascopic device, an arthroscopic device, a vascular testing device, a catheter device, a cardiac performance testing, monitoring, or measurement device, a pulmonary performance testing, monitoring, or measurement device, a vascular system performance monitoring or measurement device, a vascular system testing, monitoring, or measurement device, a metabolism monitoring or measurement device, a sonogram imaging device, a sonogram measurement device, a sonograph device, an optical response device, an optical response measurement device, an intravenous device, an arterial blood pressure measurement or monitoring device, a respiration rate measurement or monitoring device, an ultrasound imaging device, an ultrasound measurement device, a CAT SCAN device, an optical metabolism measurement or monitoring device, a radiotelemetric device, a doppler medical device, a mammogram device, a carbon dioxide detection or measurement device, a carbon monoxide detection or measurement device, a transvascular impedance measurement or monitoring device, an ultrasonic imaging device, a bone conduction device, a brain function scan analyzer device, external pulse cardiac monitoring or measurement device, a fetal heart rate measurement, monitoring, or probing, device, an endotracheal cardiac monitoring device, a finger tip blood pressure monitoring device, a psychological monitoring device, a surgical instrument, a vital signs measurement device, ear pressure regulating device, phonocardiograph device, acoustic aneurysm detector device, blood oxygen detection device, esophageal probing device, ultrasonic probing device, ausculoscope, vital signs monitoring system, heart activity monitoring device, pulmonary activity monitoring device, sphygmomanometer, esophageal stethoscope, venous pressure measuring device, differential doppler device, physiological data measuring device, body tissue movement device, breathalyzer device, camera probing device, microscopic camera probing device, and/or any other bio-metric or physiological data measuring device(s) and/or data acquisition device data acquisition device (hereinafter referred to generally or collectively as “healthcare equipment input device”, “healthcare measurement input device”, or “healthcare monitoring input device”).

The user input device 40D can also be, or can include, a global positioning device which can be utilized for determining a position or location of the user or patient communication device 40 and/or the user or operator of the same and/or any individual or healthcare patient or client who uses the same.

In another preferred embodiment, the input device 40D can also be, or can include, an interface device (I/F device) for receiving data and/or information from any one or more, or from any combination of, any pulse rate monitor or measurement device or equipment, heart rate monitor of measurement device or equipment, blood sugar monitor or measurement device or equipment, glucose monitor or measurement device or equipment, blood pressure monitor or measurement device or equipment, blood alcohol monitor or measurement device or equipment, pacemaker, defibrillator, thermometer for measuring an individual's body temperature, pulse oximeter for measuring and/or monitoring oxygen saturation and/or pulse rate, oximeter, digital finger pulse oximeter, electrocardiogram monitor or device, EKG monitor or device, electroencephalogram monitor or device, EEG monitor or device, electromyography monitor or device, EMG monitor or device, defibrillator, pacemaker, or any other device, monitor, or measurement, device or equipment, and/or any electrical or bio-medical device or equipment, which can measure an individual's physical condition, health condition, health status, healthcare condition, or physiological condition or status, or any other biological or biometric data and/or information.

In a preferred embodiment, any of the herein-described pulse rate monitors or measurement devices or equipment, heart rate monitors of measurement devices or equipment, blood sugar monitors or measurement devices or equipment, blood pressure monitors or measurement devices or equipment, blood alcohol monitors or measurement devices or equipment, pacemakers, defibrillators, thermometers, or any other devices, monitors, or measurement devices or equipment, and/or any electrical or bio-medical devices or equipment, can be wirelessly linked with and/or to the CPU 40A and/or to the user communication/personal monitoring device 40 and can be wearable, attachable to clothing, or implantable. In a preferred embodiment, the input device(s) 40D can also include a thermometer for measuring the temperature on the exterior of the user communication/personal monitoring device 40.

In preferred embodiment, any of the input device(s) 40D described or identified herein can be either connected to, or linked with, or with the CPU 40A directly or indirectly, and/or or can be wirelessly linked to or with the user communication/personal monitoring device 40, the CPU 40A, or any other component of the user communication/personal monitoring device 40 with or using a Bluetooth device or system and/or any suitable wireless device or wireless linking device or wireless linking system.

The user communication/personal monitoring device 40 can also include a display device 40E for displaying data and/or information to a user or operator. In the preferred embodiment, the display device 40E is also connected to, or linked with, the CPU 40A. The user communication/personal monitoring device 40 also includes a transmitter(s) 40F, for transmitting signals and/or data and/or information to any one or more of the central processing computer/distributed ledger/Blockchain technology system 10, to any other user communication/personal monitoring devices 40, and/or to any one or more of the provider communication device(s) 20, insurer/payer communication device(s) 30, intermediary communication devices 50, healthcare records computers 60, insurance exchange computers 70, social networking computers 80, and/or media computers 90, which may be utilized in conjunction with the present invention. In the preferred embodiment, the transmitter(s) 40F is/are also connected to, or linked with, the CPU 40A. The user communication/personal monitoring device 40 also includes a receiver(s) 40G, for receiving signals and/or data and/or information from any one or more of the central processing computer/distributed ledger/Blockchain technology system 10, from any other user communication/personal monitoring devices 40, and/or from any one or more of the provider communication device(s) 20, insurer/payer communication devices 30, intermediary communication devices 50, healthcare records computers 60, insurance exchange computers 70, social networking computers 80, and/or media computers 90, which may be utilized in conjunction with the present invention. In the preferred embodiment, the receiver(s) 10G is/are also connected to, or linked with, the CPU 10A.

The user communication/personal monitoring device 40 also includes a database(s) 40H. In the preferred embodiment, the database(s) 40H is/are also connected to, or linked with, the CPU 40A. The database(s) 40H can contain and/or can be linked to any of the non-confidential or allowable data and/or information described herein as being stored in the respective database(s) of the central processing computer/distributed ledger/Blockchain technology system 10, and/or any of the various provider communication devices 20, insurer/payer communication devices 30, intermediary communication devices 50, healthcare records computers 60, insurance exchange computers 70, social networking computers 80, and/or media computers 90, described herein.

In a preferred embodiment, the database 40H of the user communication/personal monitoring device 40 also contains any and/or all artificial intelligence (AI) software programs, software algorithms, software applications, machine language software programs, software algorithms, software applications, and/or natural language software programs, software algorithms, software applications, which are needed to required for performing any and/or all of the functions, functionalities, processing routines, and/or operations described herein as being performed by the user communication/personal monitoring device 40.

In a preferred embodiment, the database 30H also contains any and/or all of the data, information, software programs, software algorithms, software applications, which is/are described as being stored in any the respective databases in, of, or associated with, any of the various and respective user communication devices or personal monitoring devices which are disclosed and described in each of U.S. patent application Ser. No. 16/202,448, U.S. patent application Ser. No. 14/638,934, U.S. Provisional Patent Application Ser. No. 61/971,225, U.S. patent application Ser. No. 17/224,838, U.S. patent application Ser. No. 18/233,321, U.S. patent application Ser. No. 18/225,734, U.S. patent application Ser. No. 18/223,700, U.S. Pat. Nos. 7,464,040, 7,490,048, and 10,571,284, the subject matter and teachings of all of which are herein incorporated by reference in their entirety as if to be fully restated herein.

The database 40H can also contain any look-up tables, any master index tables, any data and/or information conversion information, and/or any other data and/or information, and/or software programs, software algorithms, software applications, which are needed or required for allowing the user communication/personal monitoring device 40 to perform any healthcare records interoperability operations, functions, and/or processing routines, for updating all of the various healthcare records of or associated with any of the individuals and patients who utilize the apparatus 100 of the present invention. In this regard, the user communication/personal monitoring device 40 can update all of an individual's or a patient's various and respective healthcare records of the various and respective healthcare providers of the individuals or patients, regardless of the manufacturer, source, or provider, of each respective healthcare record, upon the updating of any one healthcare record of the individual or patient. In this regard, the user communication/personal monitoring device 40 can provide healthcare record interoperability for and of all the individual's or a patient's various and respective healthcare records.

In a preferred embodiment, the database 40H can contain and/or can include any data and/or information, and/or any link or links to any data and/or information, needed or desired for enabling and/or for allowing the user communication/personal monitoring device 40 and/or the apparatus 100 and/or any of the computers or communication devices described herein as being utilized in conjunction with the apparatus 100 and methods of the present invention to perform any and/or all of the functionality described herein which is capable of being performed by the user communication/personal monitoring device 40 and/or the apparatus 100 of the present invention.

In a preferred embodiment, the database 40H can contain and/or can include any software, software programs, algorithms, or software applications (“apps”) needed or desired for enabling and/or for allowing the user communication/personal monitoring device 40 and/or the apparatus 100 and/or any of the computers or communication devices described herein as being utilized in conjunction with the apparatus 100 and methods of the present invention to perform any and/or all of the functionality or functionalities described herein which is/are capable of being performed by the user communication/personal monitoring device 40 and/or the apparatus 100 of the present invention.

In a preferred embodiment, the database(s) 40H can contain and/or can include any data and/or information regarding the user or the individual, or any users or individuals, who or which utilize the user communication/personal monitoring device 40. Any data and/or information regarding the user or individual who is using the user communication/personal monitoring device 40 to be monitored, can include, but is not limited to, the user's or the individual's name, address, home telephone number, e-mail address, IP address, test messaging number, text messaging address, cellular telephone number, the telephone number assigned to the user communication/personal monitoring device 40, date or birth, gender, height, weight, identification photograph, social security number or any other suitable identification information, and/or any other data and/or information regarding the user of the individual.

The database 40H can also contain and/or can include any data and/or information regarding the user's or the individual's user communication/personal monitoring device 40 which can include, but which is not limited to, the type of device, such as for example, a cellular telephone, Smartphone, smartphone, personal digital assistant, or any other device described herein which can be utilized as a user communication/personal monitoring device 40, the manufacturing and model number of the user communication/personal monitoring device 40, a serial number of the user communication/personal monitoring device 40, any other identifying data and/or information assigned to, associated with, or relating to, the user communication/personal monitoring device 40, the telephone number associated with, or assigned to, the user communication/personal monitoring device 40, the cellular telephone number associated with, or assigned to, the user communication/personal monitoring device 40, the wireless or mobile telephone number associated with, or assigned to, the user communication/personal monitoring device 40, an IP address associated with, or assigned to, the user communication/personal monitoring device 40, an email address associated with, or assigned to the user communication/personal monitoring device 40, a text messaging number associated with, or assigned to, the user communication/personal monitoring device 40, and/or any other data and/or information associated with the user communication/personal monitoring device 40. The database 40H can also contain and/or can include any of the above-described information for any other user communication/personal monitoring device 40 or any number of user communication/personal monitoring devices 40 which is/are used by the user or individual.

The database 40H can also contain and/or can include any data and/or information regarding a person who is monitoring the user or the individual. In a preferred embodiment, the a person who is monitoring the user or the individual can be, but is not limited to, the user's or individual's parent or parents, spouse, sibling, relative, friend, nanny, au pair, caregiver, teacher, employer, or any other person or entity, who or which is monitoring the user or individual (hereafter also referred to as “monitoring person”). For each monitoring person and, therefore, for each respective parent, spouse, sibling, relative, friend, nanny, au pair, caregiver, teacher, employer, or any other person or entity, who monitors the user of the individual or of or for the user or individual, the database 40H can contain and/or can include any data and/or information regarding the name, address, telephone number, cellular telephone number, user communication device telephone number or cellular telephone number, e-mail address, text messaging number, and/or any other data and/or information, and/or contact information, of, for, or regarding, the monitoring person. The database 40H can also contain and/or can include any other data and/or information, described herein as being stored for or regarding the user or individual, for or regarding the monitoring person.

The database 40H can also contain and/or can include any data and/or information regarding places, locations, or venues, to which the user or individual travels. The database 40H can also contain and/or can include any data and/or information regarding the daily schedule or daily schedules of or for the user or the individual, and/or any data and/or information regarding the daily routine or daily routines of or for the user or individual, any places where the user or individual is or has to be at a given time, and/or any other data and/or information regarding the user's or the individual's daily routines, weekly routines, travel routines, travel routes used, alternate travel routes used, travel times and/or time of travel regarding any travel by the user or individual, and/or any other data and/or information regarding the user's or the individual's routines that can be utilized in performing a personal monitoring service for or regarding the user or the individual.

For example, in the case of a child being monitored, the database 40H can contain and/or can include any data and/or information regarding the daily weekday schedule for the child such as, for example, the child's home address, the time or approximate time when the child leaves home for school, a preferred travel route the child takes to go to school, any alternate travel routes to the school, the time or the approximate time the child arrives at school or the time school starts for the child, the time or the approximate time the child leaves school or the time school ends for the day, a travel route to an after school activity, if applicable, a travel route to the after school activity, an alternate travel route to the after school activity, a time or an approximate time of a travel to an after school activity, a time or an approximate time when the child leaves the venue of the after school activity, a travel route from the venue of the after school activity to the child's home, a travel route to the child's home, an alternate travel route to the child's home, and/or a time or an approximate time when the child is expected to arrive at home.

As and for another example, in the case of an adult of any age being monitored, the database 40H can contain and/or can include any data and/or information regarding the daily weekday schedule for the adult such as, for example, the adult's home address, the time or approximate time when the adult leaves home for work or some other activity or venue, a preferred travel route the adult takes to go to work or some other activity or venue, any alternate travel routes to the work, activity, or venue, the time or the approximate time the adult arrives at work, the activity, or the venue, the time or the approximate time the adult leaves work, the activity, or the venue, a travel route to another activity or venue, if applicable, a travel route to the other activity or venue, an alternate travel route to the other or venue, a time or an approximate time of a travel to the other activity or venue, a time or an approximate time when the adult leaves the other activity or venue, a travel route from the other activity or venue back to the adult's home, a travel route to the adult's home, an alternate travel route to the adult's home, and/or a time or an approximate time when the adult is expected to arrive at home.

The database 40H can also contain and/or can include any data and/or information regarding any school(s), workplace(s), club(s), activity venue(s), recreational venue(s), entertainment venue(s), or any other place(s), location(s), and/or other venue(s), of the user or individual and/or to which the user or individual travels and/or at which the user or individual is known to spend time. The database 40H can also contain and/or can include any data and/or information regarding the location(s) of any school(s), workplace(s), club(s), activity venue(s), recreational venue(s), entertainment venue(s), or any other place(s), location(s), and/or other venue(s), of the user or individual, which data and/or information can include the name, the address, the telephone number, the website address, the IP address, a description of same, schedule information of or for same, and/or any other information regarding same.

The database 40H can also contain and/or can include any data and/or information regarding any weekday or weekend day schedules or itineraries of the user or individual. The database 40H can also contain and/or can include any data and/or information regarding emergency contacts for the user or the individual, including, for each emergency contact individual, his or her name, telephone number, cellular telephone number, text messaging number, e-mail address, or IP address, or any other information.

The database 40H can also contain and/or can include any data and/or information regarding any personal monitoring accounts the user or individual has or is associated with. The database 40H can also contain and/or can include any data and/or information regarding any persons responsible for monitoring the user or the individual including for each person, his or her name, telephone number, cellular telephone number, text messaging number, e-mail address, or IP address, or any other information. The database 40H can also contain and/or can include any data and/or information regarding any and/or all user communication/personal monitoring devices 40 assigned to or associated with the user or individual and/or can contain and/or can include data and/or information regarding with any the user's or individual's personal monitoring accounts.

The database 40H can also contains and/or can include any data and/or information regarding any of the central processing computer/distributed ledger/Blockchain technology systems 10, provider communication device(s) 20, insurer/payer communication devices 30, intermediary communication devices 50, healthcare records computers 60, insurance exchange computers 70, social networking computers 80, and/or media computers 90, described herein, and/or can contain and/or can include any link(s) or hyperlink(s) to same.

The database 40H can also contain and/or can include an electronic healthcare record of the user or the individual and/or any data and/or information regarding same or contained in same, any data and/or information which may be contained in the electronic healthcare record of the user or the individual, any personal healthcare record of the user or individual, any data and/or information which may be contained in the personal healthcare record of the user or the individual, any healthcare information regarding the user or the individual, any information regarding any healthcare condition or special needs of the user or the individual, any information regarding any medicines, prescribed medications, drugs, or prescribed drugs, which are needed by the user or the individual, any information regarding any allergies of the user or the individual, or any other information regarding any healthcare conditions, needs, or treatments, of, for, or regarding, the user or the individual. The database 40H can also contain and/or can include a link or hyperlink to the healthcare records computer 60 and/or to the user's or the individual's electronic healthcare record stored therein.

The database 40H can also contain and/or can include any data and/or information regarding the daily schedule for each weekday or each weekend day for the user or the individual, travel routes traveled for each day and trip, and/or time(s) associated with each trip or travel segment of each trip.

The database 40H can also contain and/or can include any data and/or information regarding the name, address, telephone number(s), cellular telephone number(s), mobile or wireless telephone number(s), e-mail address(es), text messaging address(es), IP address(es), or any other contact information for or regarding any herein-described monitoring person of or for the user or the individual or for each or any parent, sibling, family member, relative, caregiver, nanny, au pair, healthcare provider(s), healthcare insurer(s) or healthcare payer(s), or any other person or entity of or for the user or the individual.

It is important to note, as used throughout the application, the term or phrase “text messaging number” includes any and all kinds of messaging numbers, including, but not limited to, a text messaging number, an SMS messaging number, and MMS messaging number, or any other number, address, or identifier, used or needed in order to send a text message or any other message to any user, individual, person, or entity who or which uses the apparatus 100 of the present invention.

The database 40H can also contain and/or include any software, software program(s), algorithm(s), or software applications (“apps”), such as those known by those skilled in the art at the time of the filing of this application, which can allow the user communication/personal monitoring device 40 to ascertain, determine, locate, and/or display, a location or position of any user communication/personal monitoring device 40 associated with any user or individual, including, but not limited to, any user or individual who is to be monitoring the user or individual who is using or who is associated with the user communication/personal monitoring device 40, or any other authorized or designated user or individual who might be able to provide assistance to the user or individual who is using or who is associated with the user communication/personal monitoring device 40.

The database 40H can also contain and/or can include navigation software for allowing the user communication/personal monitoring device 40 to calculate travel routes from one place or point to another, to detect departures from a travel route and to re-calculate another travel route, and/or for allowing the user communication/personal monitoring device 40 to calculate and/or to store travel routes and/or any data and/or information regarding same which are used by the user or individual as well as alternate travel routes for same. The database 40H can also contain and/or can include any data and/or information regarding any allowed travel routes for the user or individual as well as disallowed travel routes for the user of individual. The database 40H can also contain and/or include map data and/or map information including, but not limited to, digitized map data and/or information and/or data and/or information for updating any map data and/or information.

The database 40H can also contain and/or include data and/or information regarding travel records for the user or individual which can contain and/or include data and/or information regarding a date and time of travel and/or travel routes taken or traveled by, and/or any other data and/or information regarding, the user or individual for or during any period of time or during and/or for or relating to any schedule or routine.

The database 40H can also contain and/or include a pre-recorded audio message(s) and/or a pre-recorded audio and video message(s), which can be recorded by any person authorized to monitor the user or individual and which can provided via the user communication/personal monitoring device 40 at any time and/or for any reason. For example, an audio and/or an audio and video recording can be played via the user communication/personal monitoring device 40 in order to assist, calm, or comfort, a lost child, to help re-orient a child, to give or provide the child or any one with whom the child comes into contact with, instructions, contact information, emergency contact information, directions, or any other information, and/or can be played to clam, comfort, or assist, a lost, disoriented, or ill, adult or child of any age, or to give or provide the child or any one with whom the child comes into contact with, instructions, contact information, emergency contact information, directions, or any other information.

The database 40H can also contain and/or can include any data, information, software, software programs, algorithms, and/or software applications (or “apps”) which are needed or desired for allowing the user communication/personal monitoring device 40 to perform any and/or all of the functions and/or functionality described herein as being capable of being performed by same and/or by the apparatus 100 and method of the present invention.

The database 40H can also contain and/or can include any of the data and/or information described herein as being stored in any of the databases of the central processing computer/distributed ledger/Blockchain technology systems 10, provider communication device(s) 20, insurer/payer communication devices 30, intermediary communication devices 50, healthcare records computers 60, insurance exchange computers 70, social networking computers 80, and/or media computers 90, and/or the RFID tags/RFID reader system(s) 92, described herein.

The database 40H can contain and/or be linked to any of the data and/or information described herein as being stored in the database 110H of the central processing computer component 10A of the central processing computer/distributed ledger/Blockchain technology system 10.

The database 40H can also contain, for the individual or patient, the individual's personal healthcare record, and/or any and/or all of the electronic healthcare records, electronic healthcare files, electronic medical records, electronic dental records, electronic pharmacy records, and/or electronic behavioral health records, and any and/or all data and/or information contained therein, of and the individual or patient.

The user communication/personal monitoring device 40 also includes an output device(s) 401 for outputting any of the data, information, and/or reports, described herein as being generated by or via the user communication/personal monitoring device 40. In the preferred embodiment, the output device(s) 401 can be a display screen, a speaker, a printer, a display of any type or kind, an indicator light, a transmitter, a modem, and/or any other device which can be used to output data or information of any kind or type. In the preferred embodiment, the output device(s) 401 can also include a beacon or a homing beacon which can transmit or provide a signal, a distress signal, or any other indication, from the user communication/personal monitoring device 40 which can be utilized determine the position, location, and/or movement, of the user communication/personal monitoring device 40. In the preferred embodiment, the output device(s) 401 is/are also connected to, or linked with, the CPU 40A.

The user communication/personal monitoring device 40 also includes a video and/or audio recording device(s) 40J which can include a camera and/or a video recording device for recording pictures or video and/or video clips and/or a microphone or an audio recording device for recording audio or audio clips which can be recorded by, and/or transmitted live, by or from the user communication/personal monitoring device 40, or which can be recorded by, and stored at or in, the user communication/personal monitoring device 40 for transmission by or from the user communication/personal monitoring device 40 at a later time. The video and/or audio recording device(s) 40J can be utilized to facilitate video conferencing, video chatting, and/or audio conferencing, and/or video and audio conferencing, between users of the user communication/personal monitoring device 40 and any user of any other computer or communication device 10, 20, 30, 50, 60, 80, and/or 90, described herein. In the preferred embodiment, the video and/or audio recording device(s) 40J is/are also connected to, or linked with, the CPU 40A.

With reference to FIG. 5, the user communication/personal monitoring device 40 can also include a global positioning system (GPS) device 40K which can be utilized to determine or ascertain the location or position of the user communication/personal monitoring device 40 at any time and/or to track movement of the user communication/personal monitoring device 40. In a preferred embodiment, the global positioning system (GPS) device 40K can be connected to, or linked with, the CPU 40A.

The user communication/personal monitoring device 40 also includes device functional equipment systems or devices 40L, which can include any the necessary communications systems or devices which are typically found in cellular telephones or wireless telephones and/or which can allow the user communication/personal monitoring device 40 to function as a cellular telephone or a wireless telephone. In a preferred embodiment, the functional equipment systems or devices 40L can also include a global positioning system (GPS) device which can be utilized to determine or ascertain the location or position of the user communication/personal monitoring device 40 at any time and which can be also be used to track movement of the user communication/personal monitoring device 40.

In a preferred embodiment, the functional equipment systems or devices 40L can also include navigation equipment or devices which are typically found in navigation devices or equipment and which can be utilized to allow the user communication/personal monitoring device 40 to function and/or to operate as a GPS equipped navigation device. In a preferred embodiment, the user communication/personal monitoring device 40 can function as a stand alone navigation device, meaning that it can perform any and/or all needed and desired navigation tasks and functions without any interaction with an external computer or device, and/or without having to access a computer over any communication network in order to obtain navigation data, information, and/or instructions, for providing navigation data, information, and/or instructions, to a user of the user communication/personal monitoring device 40.

In another preferred embodiment, the functional equipment systems or devices 40L can also include a “kill” switch or associated hardware and/or software for disabling and/or deactivating the user communication/personal monitoring device 40 in instances when same might be lost or stolen, so as to prevent its use by another person and/or to prevent any access to any data and/or information stored therein, thereby rendering the user communication/personal monitoring device 40 useless to another person after being reported, or discovered as being, lost or stolen.

In another preferred embodiment, the user communication/personal monitoring device 40 can also operate in conjunction with an external computer or device in order to obtain navigation data, information, and/or instructions, so as to provide same to a user of the user communication/personal monitoring device 40. In another preferred embodiment, the user communication/personal monitoring device 40 can process navigation data, information, and/or instructions, on its own as well as receive at least some navigation data, information, and/or instructions, from an external computer or device, in order to provide navigation data, information, and/or instructions, to a user of the user communication/personal monitoring device 40. In a preferred embodiment, the functional equipment systems or devices 40L are also connected to, or linked with, the CPU 40A.

In a preferred embodiment, the functional equipment systems or devices 40L can also include any combination of hardware and/or software for disabling the on/off switch of the user communication/personal monitoring device 40, so that the user communication/personal monitoring device 40 cannot be shut-off, or so that no operation or function of the user communication/personal monitoring device 40 can be terminated, and/or so that a telephone call, a telephone communication link, or a communication line or link, cannot be turned off or terminated, by or at the user communication/personal monitoring device 40.

In this regard, in the case of an emergency, no telephone call and/or communication line or link between the user communication/personal monitoring device 40 and any other user communication/personal monitoring device 40, or any telephone or cellular telephone, and/or any of the herein-described and respective computers or communication devices 10A, 20, 30, 50, 60, 80, and/or 90, can be terminated at or by the user communication/personal monitoring device 40, so that a communication line, link, or channel can always be maintained with the user communication/personal monitoring device 40.

In this regard, for example, if a child is lost, a telephone call and/or communication line, link, or channel, with and between the child and his or her parent can be maintained without the risk of the call being terminated at or by the user communication/personal monitoring device 40. In this regard, the parent can continue to speak with and communication with the child, can obtain position or location information from or via the user communication/personal monitoring device 40, can track the user communication/personal monitoring device 40, and/or can obtain any other information from and/or via the user communication/personal monitoring device 40, without losing contact with the child and/or his or her user communication/personal monitoring device 40.

In a preferred embodiment, the functional equipment systems or devices 40L can also include any combination of hardware and/or software for allowing the user communication/personal monitoring device 40 and any components or devices therein or associated therewith to be remotely accessed, controlled, and/or monitored, by or using any other and authorized user communication/personal monitoring device 40 used by an authorized user or individual, and/or the user communication/personal monitoring device 40 can be remotely accessed, controlled, and/or monitored, by or using any authorized central processing computer component 10A, provider communication device 20, insurer/payer communication device 30, intermediary communication device 50, healthcare records computer 60, and/or insurance exchange computer 70.

In a preferred embodiment, the user communication/personal monitoring device 40 can also include an RFID tag 92A and an RFID reader 92B which can be attached to, connected to, located on, or located within or inside a housing of, the user communication/personal monitoring device 40. In a preferred embodiment, the RFID tag 92A and/or the RFID reader can be connected to, or linked with, the CPU 40A.

In a preferred embodiment, the user communication/personal monitoring device 40, and the apparatus 100 of the present invention can be utilized in connection with human beings as well as pets and/or other non-human animals.

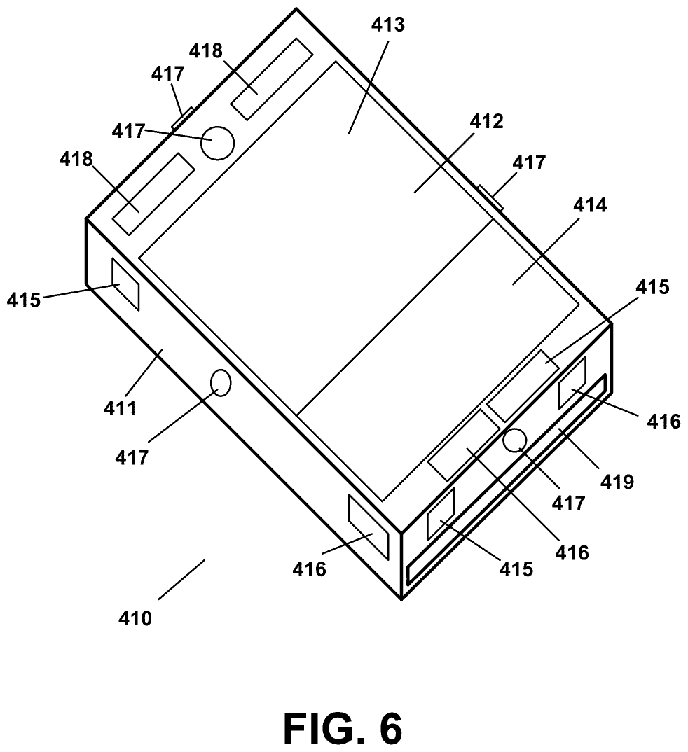

FIG. 6 illustrates another preferred embodiment of the user communication/personal monitoring device 40 of the apparatus of FIG. 5, which is denoted generally by the reference numeral 410, in a three-dimensional perspective view. In a preferred embodiment, the user communication/personal monitoring device 410 can be, or can be implemented in or with, a cellular telephone, a Smartphone, a smartphone, or a personal digital assistant, a digital watch, a smart watch, and/or any other suitable device which can be equipped with all of the necessary hardware and software needed to perform all of the functions and functionality described herein as being performed by the user communication/personal monitoring device 410 of the present invention. In another preferred embodiment, the user communication/personal monitoring device 410 can be designed to be of any size or shape, and/or the user communication/personal monitoring device 410 can be implemented using a watch, a wristwatch, a necklace, a bracelet, an ankle bracelet, an animal collar, a pet collar, a ring, or any other article of jewelry or article of clothing, or the user communication/personal monitoring device 410 can be secured to a belt, a necklace, a bracelet, an ankle bracelet, eyeglasses, a watch, a wristwatch, and/or a collar, and/or can be attached to, secured to, or placed inside or within, any article of clothing, a jacket, a coat, a shirt, a blouse, a dress, a skirt, a pair of pants, shoes, sneakers, boots, a hat, gloves, socks, stockings, a tie, a scarf, and/or an clothing or covering for any pet or animal, or any other wearable item or accessory. In this regard, and depending of the use, application, or deployment, of the user communication/personal monitoring device 410 in any given setting or situation, the user communication/personal monitoring device 410 can be designed and/or configured to be of any size, shape, type, or kind, of device.

With reference to FIG. 6, in a preferred embodiment, the user communication/personal monitoring device 410 can include a housing 411, a display screen 412 which can be a component of the display device 40E. In the preferred embodiment, the display screen 412 can be of the touch screen type or kind and can be utilized to view and to input data, information, messages, or instructions. In a preferred embodiment, the display screen 412 can include a display section 413 and a keyboard section 414. In a preferred embodiment, the keyboard section 414 can be called upon when needed and can also be dispensed with when not being used so as to facilitate the use of the entirety of the display screen 412 when desired.

In a preferred embodiment, the user communication/personal monitoring device 410 can also be equipped with a flashlight, a flashlight bulb, or with any suitable software application which can turn the display screen 412, or any portion of the display screen 412, into a flashlight. In this regard, the personal monitoring device 410 can be equipped with a flashlight or a flashlight functionality.

With reference once again to FIG. 6, the user communication/personal monitoring device 410 can also include one or more microphones 415, which can be a component of the video and/or audio recording device(s) 40J and/or the input device 40D and, which can be located on one or more, or on any or all surfaces of the user communication/personal monitoring device 410 in order to allow for any user or other individual to utilize the user communication/personal monitoring device 410 to communicate with others, to allow others to monitor audio and/or sounds at or in the vicinity of the user communication/personal monitoring device 410, and/or to allow one to use, control an operation of, to enter voice commands into, and/or to record audio information or an audio clip with and/or using, the user communication/personal monitoring device 410, and/or to allow one to simply utilize the user communication/personal monitoring device 410 to communicate with another individual or entity in a hand-free mode of operation. Any number of microphones 415 can be utilized in connection with the user communication/personal monitoring device 410.

With reference once again to FIG. 6, the user communication/personal monitoring device 410 can also include one or more speakers 416, which can be a component of the output device 401 and, which can be located on one or more, or on any or all, surfaces of the user communication/personal monitoring device 410 in order to provide audible data, information, instructions, or communications, to any user, or individual who may be using, or who may be in the vicinity of, the user communication/personal monitoring device 410.

With reference once again to FIG. 6, the user communication/personal monitoring device 410 can also include one or more cameras 417, which can be a component of the video and/or audio recording device(s) 40J and/or the input device 40D and, which can be located on one or more, or on any or all, surfaces of the user communication/personal monitoring device 410 in order to take or record a picture, a photograph, or an image, and/or to record video information or a video clip, with the user communication/personal monitoring device 410, to record a picture, a photograph, or an image, and/or to record video information or a video clip, of a user of the user communication/personal monitoring device 410 or any individual using the user communication/personal monitoring device 410 or in the vicinity of the user communication/personal monitoring device 410, to record a picture, a photograph, or an image, and/or to record video information or a video clip, of a vicinity in which the user communication/personal monitoring device 410 is located or of a surrounding of same, and/or to allow a user or any individual to engage in a video conference or video chat with another individual or other individuals using the user communication/personal monitoring device 410.

In a preferred embodiment, any one or more cameras 417 can be a wide angle lens camera or a camera having a wide angle lens for obtaining maximum viewing area. In a preferred embodiment, any one or more cameras 417 can also be a night vision camera, an infrared camera, or a camera equipped with, or utilized in connection with, night vision capability.

With reference once again to FIG. 6, the user communication/personal monitoring device 410 can include a plurality of indicator lights 418, one of which can be used to provide an indication that the user of the user communication/personal monitoring device 410 is located with a “safe” zone of travel and the other which can be used to provide an indication that the user of the user communication/personal monitoring device 410 is located outside of a “safe” zone of travel, is lost, is ill, is possibly the victim of foul play, or is otherwise in need of help or assistance. In a preferred embodiment, the indicator light 418 which is used to indicate the that user in within his or her “safe” zone can be a green light when illuminated, and the indicator light 418 which is used to indicate the that the user outside his or her “safe” zone of travel, is lost, is ill, is possibly the victim of foul play, or is otherwise in need of help or assistance, can be a red light when illuminated. In another preferred embodiment, the indicator lights 418 can be provided via the display screen or in or via a portion or section of the display screen 412.

In a preferred embodiment, any number of cameras 417, microphones 415, and/or speakers 416 can be utilized and positioned in any appropriate locations on and about the user communication/personal monitoring device 410 and/or the housing 411 of the same.

With reference once again to FIG. 6, the housing 411 is formed so as to include a hollow channel 419, as shown in FIG. 6, which runs the length of the housing 411. In a preferred embodiment, the hollow channel 419 can be utilized to attach the housing 411 of the user communication/personal monitoring device 410 to a necklace, belt, bracelet, ankle bracelet, ankle bracelet, collar, pet collar, animal collar, or other wearable device, so as to attach and/or to mount the user communication/personal monitoring device 410, or the housing 411, to the same.

In another preferred embodiment, the user communication/personal monitoring device 410 can also include any other suitable attachment device or element (not shown) which is attached or connected to, or linked with, the housing 411 and/or the user communication/personal monitoring device 410 and which can be used to secure, to mount, or to otherwise attach, the user communication/personal monitoring device 410 to, on, or in, a belt, an article of clothing, a watch, a wristwatch, a necklace, a bracelet, a collar, a pet collar, an animal collar, eyeglasses, an accessory of any type or kind, a jacket, a coat, a shirt, a blouse, a dress, a skirt, a pair of pants, shoes, sneakers, boots, a hat, gloves, socks, stockings, a tie, a scarf, or any other wearable item or accessory.

In a preferred embodiment, the user communication/personal monitoring device 410, its housing 411, and its various component parts described herein, can be constructed or rugged materials in order to protect the user communication/personal monitoring device 410 against impacts. In a preferred embodiment, the user communication/personal monitoring device 410, its housing 411, and its various component parts described herein, can also be sealed, in any appropriate manner, so as to provide for a user communication/personal monitoring device 410 which can be waterproof. In a preferred embodiment, the user communication/personal monitoring device 410 can also be designed and manufactured to as to include any suitable or buoyant material(s) which can allow the user communication/personal monitoring device 410 to float on water. In a preferred embodiment, the housing 411, or any portion or component of same, can also include made with or from, or can contain a phosphorescent material so that the housing 411, or any portion of the housing 411, of the user communication/personal monitoring device 410 can glow-in-the dark or otherwise exhibit glow-in-the-dark or luminescent properties.

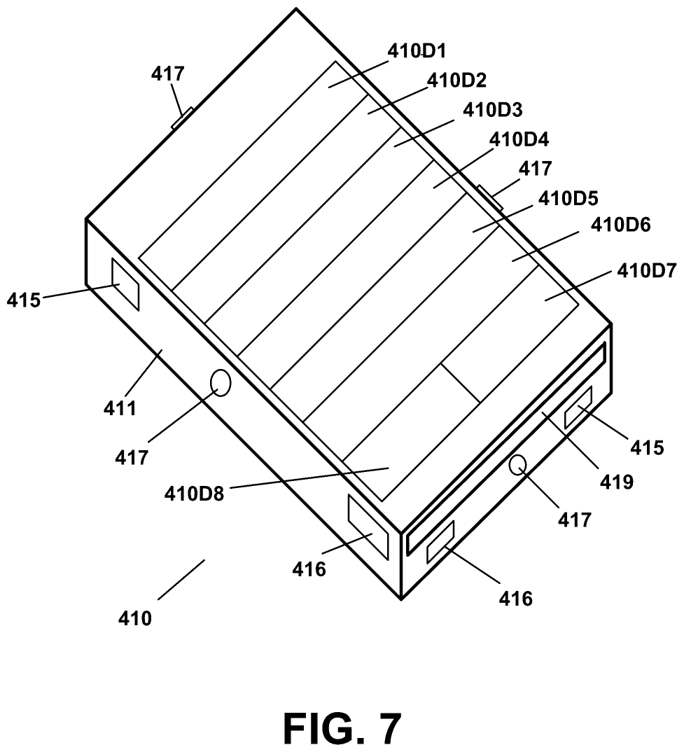

FIG. 7 illustrates an inverted view of the user communication/personal monitoring device 410 of FIG. 7 illustrating the underside of the same. With reference to FIG. 7, the user communication/personal monitoring device 410 also includes, on its underside, so as to be adjacent to a body part of the individual, person, pet, or other animal, in connection with which the user communication/personal monitoring device 410 is being utilized, an array of one or more biometric sensors or physiological sensors, leads, and/or other contact elements and/or conductive contact elements, which can include, but which is not limited to, a pulse rate monitor or sensor 410D1, a heart rate monitor or sensor 410D2, a blood sugar monitor or sensor 410D3, a blood pressure monitor or sensor 410D4, a blood alcohol monitor or sensor 410D5, a thermometer 410D6, a pacemaker or defibrillator 410D7, and/or any other biometric or physiological measurement monitor or sensor 410D8. In a preferred embodiment, the biometric or physiological measurement monitor or sensor 410D8 can include a blood oxygen sensor, a pulse oximeter sensor, or an electrocardiogram monitor or EKG sensor.

In a preferred embodiment, each of the respective intermediary computers 50, healthcare records computer 60, insurance exchange computers 70, social networking computers 80, and/or media computers 90, can be any suitable personal computer, a network computer or computer system, server computer, or any other computer or communication device, and can include a central processing unit or CPU, a random access memory device(s) (RAM), a read only memory device(s) (ROM), one or more input devices, a display device, a transmitter, a receiver(s), a database, an output device, a video and/or audio recording device(s), and/or any other suitable components and/or devices.

In a preferred embodiment, each of the respective intermediary computers 50, healthcare records computer 60, insurance exchange computers 70, social networking computers 80, and/or media computers 90, can include any and/or all of the components of the respective intermediary computers 50, healthcare records computer 60, insurance exchange computers 70, social networking computers 80, and/or media computers 90, which are described in each of U.S. patent application Ser. No. 16/202,448, U.S. patent application Ser. No. 14/638,934, U.S. Provisional Patent Application Ser. No. 61/971,225, U.S. patent application Ser. No. 17/224,838, U.S. patent application Ser. No. 18/233,321, U.S. patent application Ser. No. 18/225,734, U.S. patent application Ser. No. 18/223,700, U.S. Pat. Nos. 7,464,040, 7,490,048, and 10,571,284, the subject matter and teachings of all of which are herein incorporated by reference in their entirety as if to be fully restated herein.

In a preferred embodiment, each of the respective intermediary computers 50, healthcare records computer 60, insurance exchange computers 70, social networking computers 80, and/or media computers 90, can be specially programmed to, and can perform any and/or all of the functions, functionalities, and/or operations of the respective intermediary computers 50, healthcare records computer 60, insurance exchange computers 70, social networking computers 80, and/or media computers 90, which are described in each of U.S. patent application Ser. No. 16/202,448, U.S. patent application Ser. No. 14/638,934, U.S. Provisional Patent Application Ser. No. 61/971,225, U.S. patent application Ser. No. 17/224,838, U.S. patent application Ser. No. 18/233,321, U.S. patent application Ser. No. 18/225,734, U.S. patent application Ser. No. 18/223,700, U.S. Pat. Nos. 7,464,040, 7,490,048, and 10,571,284, the subject matter and teachings of all of which are herein incorporated by reference in their entirety as if to be fully restated herein.

The apparatus and method of the present invention can be utilized in numerous preferred embodiments in order to provide a vast array of healthcare and healthcare-related services for any one or more of the various parties described herein. While certain of the preferred embodiments may be described with regards to utilization by a particular party, it is important to note that any individual, patient, user, provider, insurer, payer, and/or intermediary or third party, may utilize the present invention in the same, similar, and/or analogous, manner. For example, a preferred embodiment for determining and/or ascertaining a medical diagnosis can be described as being utilized by a treating physician as well as can be utilized by a provider to verify and/or check a diagnosis as well as by a patient or other user or individual in order to perform a self-diagnosis or double check a doctor's diagnosis. In the same manner, any other preferred embodiment and/or other uses of the present invention can be utilized by any of the parties described herein.

The apparatus 100 of the present invention can also be utilized in order to allow an individual or a patient, or a caregiver, or one responsible for the care of an individual or patient, to own and or control his or her own personal healthcare record or electronic healthcare record(s), as well as to enter notes, comments, or messages, regarding or relating to the individual or patient into one or more of any of the individual's, patient's, or caregiver's, electronic healthcare record(s), electronic healthcare file(s), electronic medical record(s), electronic dental record(s), electronic pharmacy record(s), and/or electronic behavioral healthcare record(s), and/or personal healthcare record(s).

Any respective note, comment, or message, can be in text form, audio form, or video form, and/or any combination of the same, and can contain information regarding a symptom, an illness, an experience, a treatment, a diagnosis, a treatment plan, an activity, a problem, a concern, a thought or an idea, a question, a question for a healthcare provider, or any other information which the individual or patient, or one caring for the individual or patient, may deem important to be recorded or noted in the respective electronic healthcare record(s), electronic medical record(s), electronic dental record(s), electronic pharmacy record(s), and/or electronic behavioral healthcare record(s), and/or personal healthcare record(s), or which can be communicated to, or otherwise made available to, a provider or an insurer or payer.

A healthcare provider can access, obtain, and/or use, the information provided or contained in the note, comment, or message, or provided or contained in multiple notes, comments, or messages, for any suitable purpose, such as, but not limited to, for preparing for, or for use during, a remote or virtual office visit with, or a remote or distance examination of, an individual, a patient, or a caregiver for or an individual or a patient, during a video telephone call, a video chat session, or a videoconference, with the individual, the patient, or the caregiver for or the individual or the patient, or for use during a conversation, discussion, or consultation, during a video telephone call, a video chat session, or a videoconference, with the individual, the patient, or the caregiver for the individual or the patient, or for use during a conversation, discussion, or consultation, during a video telephone call, a video chat session, or a videoconference, with and between, the healthcare provider, and the individual, the patient, or the caregiver for or the individual or the patient, or another healthcare provider or any other provider, or an insurer or a payer, or an intermediary, or for use during reviewing, updating, modifying, or performing any other activity in connection with, an individual's or patient's healthcare records, files, or histories, for use while making a diagnosis, for use while formulating a treatment or a treatment plan, for use in reviewing or evaluating an individual's or patient's diagnosis or treatment, for use in treatment planning and/or the evaluating of same, for use in care management, for use in monitoring or evaluating a recovery, for use in providing continuing or on-going care or treatment, for use in connection with the providing of a remote healthcare services or telehealth services, and/or for any other suitable use or purpose.

Any notes, comments, or messages, can be provided by the individual, patient, or caregiver, or by any person caring for the individual or patient, while making an appointment, in advance of a video telephone call, a video chat session, or a videoconference, or a remote or virtual office visit with, or a remote or distance examination of, or in advance of a video telephone call, a video chat session, or a videoconference, or a remote or distance examination of, with the individual, the patient, or the caregiver for the individual or the patient, or a video telephone call, a video chat session, or a videoconference, with another provider, or a payer or insurer or any third party or intermediary, in connection with any telehealth related activity, or for the purpose of making and entering a note, comment, or message, into the individual's or patient's respective electronic healthcare record(s), electronic medical record(s), electronic dental record(s), electronic pharmacy record(s), and/or electronic behavioral healthcare record(s), and/or personal healthcare record(s).

In this regard, the present invention can allow an individual or patient, or a caregiver, or one responsible for caring for the individual or patient, to make and enter any notes, comments, or messages, into the individual's or patient's respective electronic healthcare record(s), electronic medical record(s), electronic dental record(s), electronic pharmacy record(s), and/or electronic behavioral healthcare record(s), and/or personal healthcare record(s), so as to facilitate accurate and complete healthcare information record keeping.

In a preferred embodiment, the apparatus 100 of the present invention can be utilized in order to provide for the interoperability of an individual's or patient's personal healthcare record(s) with any and/or all of the individual's or patient's other and respective electronic healthcare record(s), electronic medical record(s), electronic dental record(s), electronic pharmacy record(s), behavioral healthcare record(s), as well in order to provide for the interoperability of any and/or all of the individual's or patient's respective electronic healthcare record(s), electronic medical record(s), electronic dental record(s), electronic pharmacy record(s), and/or electronic behavioral healthcare record(s), and/or personal healthcare record(s), with and between each other. In this regard, the apparatus 100 of the present invention can provide a technical solution for the problems associated with any non-interoperability of and between the various and respective electronic healthcare record(s), electronic medical record(s), electronic dental record(s), electronic pharmacy record(s), and/or electronic behavioral healthcare record(s), and/or personal healthcare record(s), in the marketplace which do not provide for interoperability with other electronic healthcare records, electronic medical records, electronic dental records, electronic pharmacy records, and/or electronic behavioral healthcare records, and/or personal healthcare records.

In a preferred embodiment, the apparatus 100 of the present invention can also provide for the complete updating of any and/or all of an individual's or patient's respective electronic healthcare records, electronic medical records, electronic dental records, electronic pharmacy records, and/or electronic behavioral healthcare records, and/or personal healthcare records anytime, and in response to, any change, alteration, and/or addition of any new or additional information, to any one of the individual's or patient's respective electronic healthcare records, electronic medical records, electronic dental records, electronic pharmacy records, and/or electronic behavioral healthcare records, and/or personal healthcare records.

In a preferred embodiment, it is envisioned that an individual or patient can be assigned a personal healthcare record which can be stored on, or which can be accessed from and/or using, any and/or all of the individual's or patient's user communication/personal monitoring device(s) 40. In a preferred embodiment, the individual's or patient's can also be stored in the database 110H of the central processing computer component 10A, and/or can be stored in the respective databases 20H and 30H of the any and/or all of the provider communication device(s) 20 of or associated with any and/or all of the healthcare providers, wellness providers, and/or fitness providers of or for the individual or patient, and/or any and/or all of the insurer/payer communication devices 30 of or associated with any and/or all of the healthcare insurers or healthcare payers of or for the individual or patient.

It is envisioned that the individual's or patient's assigned personal healthcare record can follow the individual or patient for his or her lifetime and/or from the time of the assignment and/or creation of the same. In this regard, a comprehensive electronic healthcare record can be utilized in order to provide comprehensive information regarding the individual or patient to any of the authorized users, providers, insurers, payers, or other authorized individuals or entities which can access and utilize the same.

In a preferred embodiment, it is also envisioned that any and/or all of the various and respective electronic healthcare records, electronic medical records, electronic dental records, electronic pharmacy records, and/or electronic behavioral healthcare records, and/or personal healthcare records, of or for an individual or patient, and/or any data and/or information stored therein, can also be stored in the database 40H of each of the individual's or patient's user communication/personal monitoring device(s) 40, as well as can be stored in the database 110H of the central processing computer component 10A, and/or in the respective databases 20H and 30H of the any and/or all of the provider communication device(s) 20 of or associated with any and/or all of the healthcare providers, wellness providers, and/or fitness providers of or for the individual or patient, and/or any and/or all of the insurer/payer communication device(s) 30 of or associated with any and/or all of the healthcare insurers or healthcare payers of or for the individual or patient.

In this manner, the apparatus 100 of the present invention and the respective user communication/personal monitoring device(s) 40, the central processing computer component 10A, the provider communication device(s) 20, and/or the insurer/payer communication device(s) 30, can provide for the interoperability of and between any and/or all of the various and respective electronic healthcare records, electronic medical records, electronic dental records, electronic pharmacy records, and/or electronic behavioral healthcare records, and/or personal healthcare records, of or for an individual or patient.

In a preferred embodiment, any and/or all of the various and respective electronic healthcare records, electronic medical records, electronic dental records, electronic pharmacy records, and/or electronic behavioral healthcare records, and/or personal healthcare records, which can be utilized in connection with and/or in conjunction with the apparatus 100 of the present invention, can be any of the respective electronic healthcare records, electronic medical records, electronic dental records, electronic pharmacy records, and/or electronic behavioral healthcare records, and/or personal healthcare records, which are available in the marketplace as of the filing of this patent application.

In a preferred embodiment, any and/or all of the various and respective electronic healthcare records, electronic medical records, electronic dental records, electronic pharmacy records, and/or electronic behavioral healthcare records, and/or personal healthcare records, which can be utilized in connection with and/or in conjunction with the apparatus 100 of the present invention, can contain and/or can include any and/or all of the data, data fields, information, information fields, and/or data or information sections, which are currently found in, and/or currently included in, any and/or all of the electronic healthcare records, electronic medical records, electronic dental records, electronic pharmacy records, and/or electronic behavioral healthcare records, and/or personal healthcare records, which are available in the marketplace as of the filing of this patent application.

Any of the herein-described look-up tables, any master index tables, any data and/or information conversion information, and/or any other data and/or information, and/or software programs, software algorithms, software applications, which are needed or required for performing any healthcare records interoperability operations, functions, and/or processing routines, and/or for updating all of the various healthcare records of or associated with any of the individuals and patients who utilize the apparatus 100 of the present invention, can include any data and/or information regarding any and/or all of the data, data fields, information, information fields, and/or data or information sections, for and which are currently found in, and/or currently included in, any and/or all of the electronic healthcare records, electronic medical records, electronic dental records, electronic pharmacy records, and/or electronic behavioral healthcare records, and/or personal healthcare records, which are available in the marketplace as of the filing of this patent application.

Any of the herein-described look-up tables, any master index tables, any data and/or information conversion information, and/or any other data and/or information, and/or software programs, software algorithms, software applications, which are needed or required for performing any healthcare records interoperability operations, functions, and/or processing routines, and/or for updating all of the various healthcare records of or associated with any of the individuals and patients who utilize the apparatus 100 of the present invention, can also include any data and/or information correlating and/or cross-correlating, and/or cross-referencing, any and/or all of the data, data fields, information, information fields, and/or data or information sections, of, for, or regarding, any and/or all of the herein-described electronic healthcare records, electronic medical records, electronic dental records, electronic pharmacy records, and/or electronic behavioral healthcare records, and/or personal healthcare records, which are available in the marketplace as of the filing of this patent application, with all other healthcare records, electronic medical records, electronic dental records, electronic pharmacy records, and/or electronic behavioral healthcare records, and/or personal healthcare records, being utilized, and/or which can be utilized, in connection with, and/or in conjunction with, the apparatus 100.

In a preferred embodiment, each of the central processing computer component 10A, the provider communication devices 20, the insurer/payer communication devices 30, and the user communication/personal monitoring devices 40, can be equipped and programmed so as to utilize any and/or all of the data and/or information in the herein-described look-up tables, master index tables, data and/or information conversion information, and/or any other data and/or information, in order to perform and provide any and/or all of the healthcare records interoperability operations, functions, and/or processing routines, described herein, and/or for updating all of the various healthcare records, electronic healthcare records, electronic medical records, electronic dental records, electronic pharmacy records, and/or electronic behavioral healthcare records, and/or personal healthcare records, of or associated with any of the individuals and patients who utilize the apparatus 100 of the present invention as described herein.

In a preferred embodiment, the apparatus 100 of the present invention can be utilized in order to allow an individual or patient to enter information into, and/or to change, alter, or modify, data and/or information in, his or her personal healthcare record. In this embodiment, any and/or all of the individual's or patient's various healthcare records, electronic healthcare records, electronic medical records, electronic dental records, electronic pharmacy records, and/or electronic behavioral healthcare records, and/or personal healthcare records, can be updated with any and/or all of the herein-described data, information, messages, and/or reports.

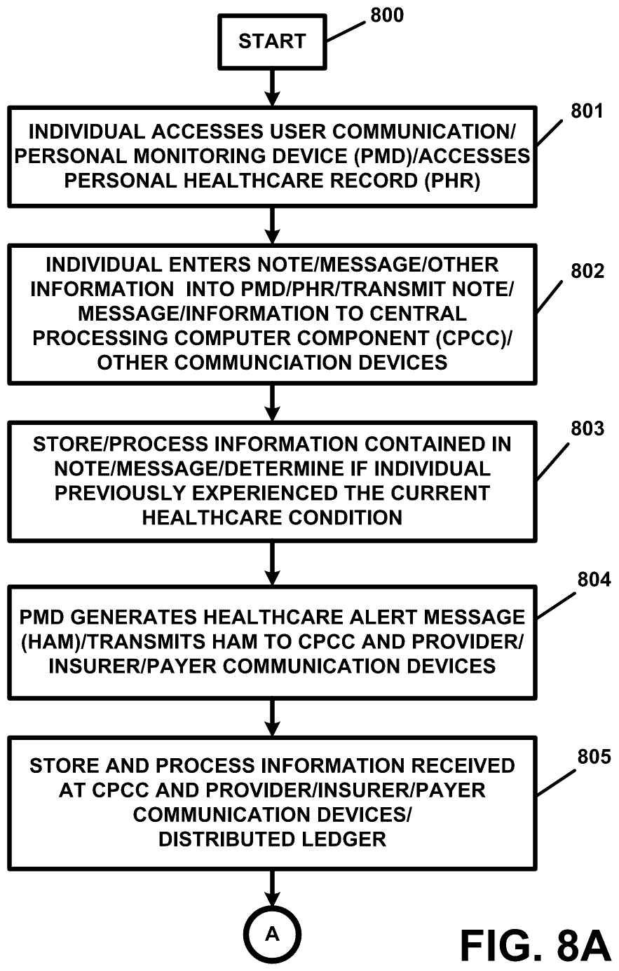

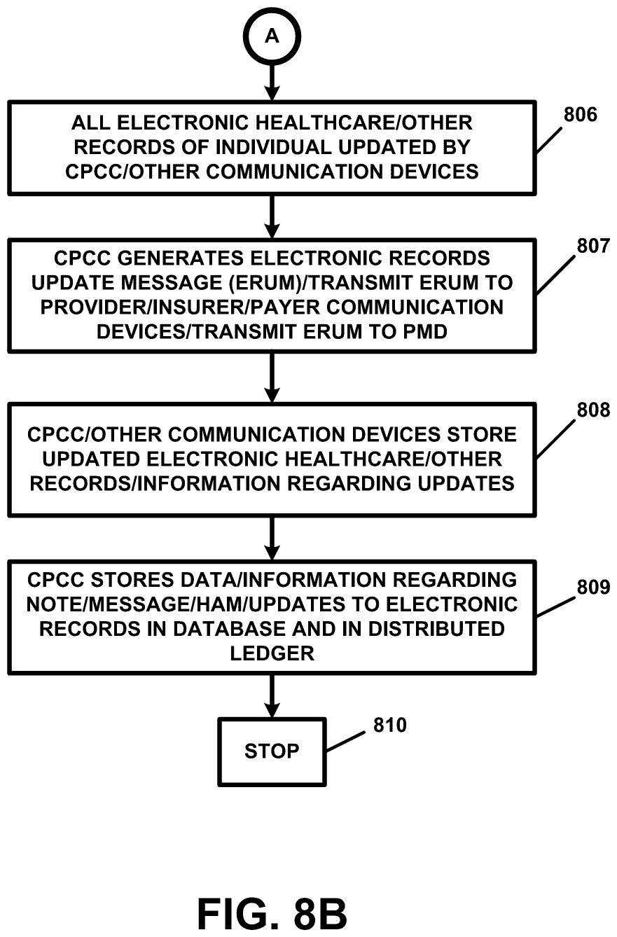

FIGS. 8A and 8B illustrate a preferred embodiment method of utilizing the apparatus 100 of the present invention, in flow diagram form. With reference to FIGS. 8A and 8B, the operation of the apparatus 100 will commence at step 800. At step 801, the individual or user can access his or her user communication/personal monitoring device 40 and can access his or her personal healthcare record. At step 802, the individual or patient can enter information into his or her personal healthcare record, and/or the individual or patient can change, alter, or modify, data and/or information in, which is already stored in his or her personal healthcare record.

At or during step 802, the individual or patient can enter a note, message, healthcare journal message, or healthcare diary message, which can be stored in his or her personal healthcare record. In a preferred embodiment, the note, message, healthcare journal message, or healthcare diary message, can be transmitted, from the user communication/personal monitoring device 40 to and stored at or in the central processing computer component 10A, can be transmitted from the user communication/personal monitoring device 40 to and stored at any provider communication device(s) 20 of any healthcare, wellness, or fitness, provider of or for the individual or patient, can be transmitted from the user communication/personal monitoring device 40 to and stored at any insurer/payer communication device(s) 30 of any healthcare insurer or healthcare payer of or for the individual or patient, and/or can be transmitted from the user communication/personal monitoring device 40 to and stored at any user communication/personal monitoring device 40 of or associated with any caregiver of or for the individual or patient.

In a preferred embodiment, the note, message, healthcare journal message, or healthcare diary message, can also be stored in the respective database 110H, 20H, 30H, and/or 40H, of the respective central processing computer component 10A, provider communication device(s) 20 of any healthcare, wellness, or fitness, provider of or for the individual or patient, insurer/payer communication device(s) 30 of any healthcare insurer or healthcare payer of or for the individual or patient, and/or user communication/personal monitoring device 40 of or associated with any caregiver of or for the individual or patient.

At step 802, the note, message, healthcare journal message, or healthcare diary message, can include text information, an audio message or voice message, a picture or pictures, a photograph or photographs, a video recording or video recordings, and/or video and/or audio clips. In a preferred embodiment, any audio message or voice message, any picture or pictures, any photograph or photographs, any video recording or video recordings, and/or any video and/or audio clips, can be taken, obtained, or recorded, by or with the video and/or audio recording devices 40J. The note, message, healthcare journal message, or healthcare diary message, can be created or generated to make a note of or regarding a healthcare condition experienced by or being experienced by the individual or patient, or can be created or generated to make a note of or regarding a state of healthcare, a state of illness, a state of on-going treatment, to make a note of or regarding a change regarding or involving a prescription drug usage, prescription, change, addition, or deletion, or to make a note of or regarding a change regarding or involving a treatment, or a treatment change, addition, or deletion, and/or to make a note of or regarding any other health information or healthcare information, or healthcare, wellness, or fitness, information regarding or relating to the individual or patient.

In a preferred embodiment, the note, message, healthcare journal message, or healthcare diary message, can also be created or generated to make a note of or regarding a measurement, or a series a series of measurements, made by any one or more of the herein-described input devices 40D, which, as noted herein, can include, but are not limited to, a pulse rate monitor or measurement device or equipment, a heart rate monitor of measurement device or equipment, a blood sugar monitor or measurement device or equipment, a glucose monitor or measurement device or equipment, a blood pressure monitor or measurement device or equipment, a blood alcohol monitor or measurement device or equipment, a pacemaker, a defibrillator, a thermometer for measuring an individual's body temperature, a pulse oximeter for measuring and/or monitoring oxygen saturation and/or pulse rate, an oximeter, a digital finger pulse oximeter, an electrocardiogram monitor or device, an EKG monitor or device, an electroencephalogram monitor or device, an EEG monitor or device, an electromyography monitor or device, an EMG monitor or device, a defibrillator, a pacemaker, or any other device, monitor, or measurement, device or equipment, and/or any electrical or bio-medical device or equipment, which can measure an individual's physical condition, health condition, health status, healthcare condition, or physiological condition or status, or any other biological or biometric data and/or information. In a preferred embodiment, any pulse rate monitor or measurement device or equipment, heart rate monitor of measurement device or equipment, blood sugar monitor or measurement device or equipment, glucose monitor or measurement device or equipment, blood pressure monitor or measurement device or equipment, blood alcohol monitor or measurement device or equipment, pacemaker, defibrillator, thermometer for measuring an individual's body temperature, pulse oximeter for measuring and/or monitoring oxygen saturation and/or pulse rate, oximeter, digital finger pulse oximeter, electrocardiogram monitor or device, EKG monitor or device, electroencephalogram monitor or device, EEG monitor or device, electromyography monitor or device, EMG monitor or device, defibrillator, pacemaker, or any other device, monitor, or measurement, device or equipment, and/or any electrical or bio-medical device or equipment, which can measure an individual's physical condition, health condition, health status, healthcare condition, or physiological condition or status, or any other biological or biometric data and/or information, can also be an input device 40D, or can be a component of any input device 40D, can be connected via a wired connection to the user communication/personal monitoring device 40, or can be connected via a wireless connection to the user communication/personal monitoring device 40D, and/or can be connected via a Bluetooth or other suitable or appropriate wireless technology.

The user input devices 40D can also be, or can include, any one or more of, and/or any combination of, a thermometer, a digital thermometer, a stethoscope, a heart rate monitor or measurement device, a pulse rate monitor or measurement device, a blood pressure monitor or measurement device, a blood oxygen or percentage oxygen measurement device, a blood pressure measurement device, a blood analysis device or machine, a respirator, a respiration monitoring or measurement device, a dialysis machine, a dialysis device, electrocardiograph (EKG) machine or device, electrocephalograph (EEG) machine or device, electromyograph (EMG) machine or device, a magnetic resonance imaging (MRI) machine or device, X-ray machine or device, medical imaging machine or device, thermal imaging machine or device, a heart sound monitor or measurement device, a lung sound monitoring or measurement device, respiration rate monitoring or measurement device, a laparascopic device, an arthroscopic device, a vascular testing device, a catheter device, a cardiac performance testing, monitoring, or measurement device, a pulmonary performance testing, monitoring, or measurement device, a vascular system performance monitoring or measurement device, a vascular system testing, monitoring, or measurement device, a metabolism monitoring or measurement device, a sonogram imaging device, a sonogram measurement device, a sonograph device, an optical response device, an optical response measurement device, an intravenous device, an arterial blood pressure measurement or monitoring device, a respiration rate measurement or monitoring device, an ultrasound imaging device, an ultrasound measurement device, a CAT SCAN device, an optical metabolism measurement or monitoring device, a radiotelemetric device, a doppler medical device, a mammogram device, a carbon dioxide detection or measurement device, a carbon monoxide detection or measurement device, a transvascular impedance measurement or monitoring device, an ultrasonic imaging device, a bone conduction device, a brain function scan analyzer device, external pulse cardiac monitoring or measurement device, a fetal heart rate measurement, monitoring, or probing, device, an endotracheal cardiac monitoring device, a finger tip blood pressure monitoring device, a psychological monitoring device, a surgical instrument, a vital signs measurement device, ear pressure regulating device, phonocardiograph device, acoustic aneurysm detector device, blood oxygen detection device, esophageal probing device, ultrasonic probing device, ausculoscope, vital signs monitoring system, heart activity monitoring device, pulmonary activity monitoring device, sphygmomanometer, esophageal stethoscope, venous pressure measuring device, differential doppler device, physiological data measuring device, body tissue movement device, breathalyzer device, camera probing device, microscopic camera probing device, and/or any other bio-metric or physiological data measuring device(s) and/or data acquisition device data acquisition device (hereinafter referred to generally or collectively as “healthcare equipment input device”, “healthcare measurement input device”, or “healthcare monitoring input device”).

Any data and/or information obtained, read by, or recorded by, any of the herein-described input devices 40 and healthcare equipment input devices, healthcare measurement input devices, or healthcare monitoring input devices, can also be contained or included in the note, message, healthcare journal message, or healthcare diary message, in any text, graphic, video, and/or audio, form, or combination of same.

The note, message, healthcare journal message, or healthcare diary message, can be created or generated for storage in the individual's or patient's personal healthcare record, and/or for transmission and communication to any healthcare, wellness, or fitness, providers of or for the individual or patient, for transmission and communication to any healthcare payer or healthcare insurer of or for the individual or patient, and/or for transmission and communication to any caregiver of or for the individual or patient. In a preferred embodiment, the note, message, healthcare journal message, or healthcare diary message, can be created or generated for note-keeping purposes, and/or to seek the advice and/or treatment, and/or any other services, from any healthcare, wellness, or fitness, provider.

At step 803, the CPU 40A and/or the user communication/personal monitoring device 40 will store the note, message, healthcare journal message, or healthcare diary message, in the user communication/personal monitoring device 40. At step 803, the CPU 40A and/or the user communication/personal monitoring device 40 will also store the data and/or information contained or included in the note, message, healthcare journal message, or healthcare diary message, in, and as a new entry into, the individual's or patient's personal healthcare record and in the database 40H. The new entry into the individual's or patient's personal healthcare record can include any and/or all text information, audio message or voice message, picture or pictures, photograph or photographs, video recording or video recordings, and/or video and/or audio clips, contained in the note, message, healthcare journal message, or healthcare diary message.

In a preferred embodiment, any and/or all of the herein-described electronic healthcare records, electronic medical records, electronic dental records, electronic pharmacy records, and/or electronic behavioral healthcare records, and/or personal healthcare records, can store any of the herein-described notes, messages, healthcare journal messages, or healthcare diary messages, and/or any and/or all text information, audio messages or voice messages, picture or pictures, photograph or photographs, video recording or video recordings, and/or video and/or audio clips, contained therein.

At step 803, the CPU 40A and/or the user communication/personal monitoring device 40 can process any and/or all of the data and/or information contained in the note, message, healthcare journal message, or healthcare diary message, and/or can process any and/or all of any data and/or information obtained, read, or recorded, by any of the herein-described input devices 40 and healthcare equipment input devices, healthcare measurement input devices, or healthcare monitoring input devices, using data and/or information already stored in the individual's or patient's personal healthcare record in order to determine whether or not the individual or patient has previously experienced the healthcare condition, or a similar healthcare condition, which is currently being experienced by the individual or patient.

In a preferred embodiment, the CPU 40A and/or the user communication/personal monitoring device 40 can perform the above-described processing function, operation, or routine, using an artificial intelligence (AI) software program, software algorithm, software application, or a machine language software program, software algorithm, software application, and/or a natural language software program, software algorithm, software application. In another preferred embodiment, the CPU 40A and/or the user communication/personal monitoring device 40 can perform the above-described processing function, operation, or routine, without using any artificial intelligence (AI) software program, software algorithm, software application, or machine language software program, software algorithm, software application, and/or natural language software program, software algorithm, software application.

In a preferred embodiment, if the CPU 40A and/or the user communication/personal monitoring device 40 determines that the individual or patient has previously experienced the healthcare condition, or a similar condition, which is currently being experienced by the individual or patient, the CPU 40A and/or the user communication/personal monitoring device 40 can generate a previous condition report containing information regarding when the individual or patient experienced the condition, the date of such, the symptoms of the condition(s) previously experienced, the healthcare provider(s), if any, from which he or she received care, any diagnosis which was made, any treatment which was administered, and/or any prescriptions for medication or drugs which may have been issued to the individual or patient, and/or any other information regarding the condition(s) which was previously experienced. In a preferred embodiment, if a previous condition report is generated, then the previous condition report will be included in the note, message, healthcare journal message, or healthcare diary message.

In a preferred embodiment, if a previous condition report is generated, then the previous condition report will be included in the note, message, healthcare journal message, or healthcare diary message. In a preferred embodiment, the user communication/personal monitoring device 40 will provide information regarding the previous condition report, the previous condition report, and/or any information regarding the same, via the display device 40E and/or via the output device 401 which can be a speaker.

At step 804, the CPU 40A and/or the user communication/personal monitoring device 40 can generate a healthcare alert message and can transmit the same to the central processing computer component 10A, and/or to any provider communication device(s) 20 of any healthcare, wellness, or fitness, provider of or for the individual or patient, and/or to any insurer/payer communication device(s) 30 of any healthcare insurer or healthcare payer of or for the individual or patient, and/or to any user communication/personal monitoring device 40 of or associated with any caregiver of or for the individual or patient. In a preferred embodiment, the healthcare alert message can include or contain any and/or all of the information contained in the note, message, healthcare journal message, or healthcare diary message, including any text information, audio message or voice message, picture or pictures, photograph or photographs, video recording or video recordings, and/or video and/or audio clips, contained therein, along with a copy of any and/or all data and/or information which is contained in the new entry made into the individual's or patient's personal healthcare record.

In a preferred embodiment, the healthcare alert message can include or contain any and/or all data and/or information contained in and/or regarding the new entry made into the individual's or patient's personal healthcare record. In a preferred embodiment, data and/or information contained in and/or regarding the new entry made in the individual's or patient's personal healthcare record can include any and/or all text information, audio message or voice messages, picture or pictures, photograph or photographs, video recording or video recordings, and/or video and/or audio clips, contained in the note, message, healthcare journal message, or healthcare diary message, as well as the note, message, healthcare journal message, or healthcare diary message itself.

At step 805, the healthcare alert message can be received by, and can be stored by, the central processing computer component 10A, and/or by any provider communication device(s) 20 of any healthcare, wellness, or fitness, provider of or for the individual or patient, and/or by any insurer/payer communication device(s) 30 of any healthcare insurer or healthcare payer of or for the individual or patient, and/or by any user communication/personal monitoring device 40 of or associated with any caregiver of or for the individual or patient. At step 805, information regarding the healthcare alert message and/or any data and/or information contained or included in the same, and/or any data and/or information relating to the same, can also be stored by the central processing computer component 10A in the database 10H and/or in the distributed ledger and Blockchain technology system component 10B of the central processing computer/distributed ledger/Blockchain technology system 10.

At step 805, any data and/or information contained in and/or regarding the new entry made into the individual's or patient's personal healthcare record can also be received by, and can be stored by, the central processing computer component 10A, and/or by any provider communication device(s) 20 of any healthcare, wellness, or fitness, provider of or for the individual or patient, and/or by any insurer/payer communication device(s) 30 of any healthcare insurer or healthcare payer of or for the individual or patient, and/or by any user communication/personal monitoring device 40 of or associated with any caregiver of or for the individual or patient. At step 805, information regarding, or information regarding information contained in, the new entry made into the individual's or patient's personal healthcare record, and/or any data and/or information contained or included in the same or relating to the same, can also be stored by the central processing computer component 10A in the database 110H and/or in the distributed ledger and Blockchain technology system component 10B of the central processing computer/distributed ledger/Blockchain technology system 10.

In a preferred embodiment, the new entry made in the individual's or patient's personal healthcare record, and/or any data and/or information contained therein or relating thereto, can include any and/or all text information, audio message or voice message, picture or pictures, photograph or photographs, video recording or video recordings, and/or video and/or audio clips, contained in the note, message, healthcare journal message, or healthcare diary message, as well as the note, message, healthcare journal message, or healthcare diary message, itself. The new entry made in the individual's or patient's personal healthcare record can also contain any data and/or information obtained, read by, or recorded by, any of the herein-described input devices 40 and healthcare equipment input devices, healthcare measurement input devices, or healthcare monitoring input devices, and can also contain information regarding and/or contained or included in the note, message, healthcare journal message, or healthcare diary message, in any text, graphic, video, and/or audio, form, or combination of same.

At step 806, the central processing computer component 10A will update any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, of or for the individual or patient, with and so as to include any and/or all of the data and/or information, contained in and/or regarding the note, the message, the healthcare journal message, or the healthcare diary message, and/or contained in or regarding the healthcare alert message, and/or contained in or regarding the new entry made into the individual's or patient's personal healthcare record.

In this regard, the central processing computer component 10A will update any, each and every, and/or all, of the various and respective provider's electronic healthcare record(s), electronic medical record(s), electronic dental record(s), electronic pharmacy record(s), and/or electronic behavioral healthcare record(s), of, for, or associated, the individual or patient, with and so as to include any and/or all of the data and/or information, contained in or regarding the note, the message, the healthcare journal message, or the healthcare diary message, and/or contained in or regarding the healthcare alert message, and/or contained in or regarding the new entry made into the individual's or patient's personal healthcare record.

In a preferred embodiment, the central processing computer component 10A can update any, each and every, and/or all, of the electronic healthcare record(s), electronic medical records(s), electronic dental record(s), electronic pharmacy record(s), and/or electronic behavioral healthcare record(s), of, for, or associated, with each healthcare provider or other authorized provider, of or for the individual or patient, by using the any of the herein-described look-up tables, master index tables, and/or data and/or information conversion information, in any appropriate and suitable manner, and by using and suitable and/or appropriate algorithms and/or software, and/or processing routines, in order to perform such updates.

As and for an example, in a preferred embodiment, at step 806, the central processing computer component 10A can process any of the data and/or information contained in the note, the message, the healthcare journal message, or the healthcare diary message, and/or contained in the healthcare alert message, and/or contained in the new entry made into the individual's or patient's personal healthcare record, so as to identify and/or sort the respective data or information into their respective data fields and information fields and thereafter compile the data and/or information into a master template. Thereafter, using information contained in the any of the herein-described look-up tables, master index tables, and/or data and/or information conversion information, the central processing computer component 10A can then insert a new entry into any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, of or for the individual or patient, as well as into the personal healthcare record of or for the individual or patient.

In this manner, each and every, and/or all, of the electronic healthcare records, electronic medical records, electronic dental records, electronic pharmacy records, and/or electronic behavioral healthcare records, and/or personal healthcare records, can be interoperable with each and every other electronic healthcare record(s), electronic medical record(s), electronic dental record(s), electronic pharmacy record(s), and/or electronic behavioral healthcare record(s), and/or personal healthcare record(s).

In the above manner, and/or in any other appropriate and/or suitable manner, at step 806, the central processing computer component 10A can update any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, of or for the individual or patient. It is important to note the central processing computer component 10A can update any, each and every, and/or all, of the electronic healthcare record(s), electronic medical record(s), electronic dental record(s), electronic pharmacy record(s), and/or electronic behavioral healthcare record(s), by and using any other suitable and/or appropriate method or methods, and/or software algorithms, software routines, and/or software programs.

At step 806, any of the herein-described provider communication devices 20, insurer/payer communication devices 30, and/or the individual's or patients user communication/personal monitoring device(s) 40, can also perform the very same operations as the central processing computer component 10A in order to update any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, with any of the herein-described data and/or information contained in the note, the message, the healthcare journal message, or the healthcare diary message, and/or contained in the healthcare alert message, and/or contained in the new entry made into the individual's or patient's personal healthcare record.

At step 807, the central processing computer component 10A will generate an electronic records update alert message and transmit the same, along with the update(s) to, and/or along with information contained in or regarding the update(s) to, and/or along with the updated and respective electronic healthcare record(s), electronic medical record(s), electronic dental record(s), electronic pharmacy record(s), and/or electronic behavioral healthcare record(s), and/or personal healthcare record, to each of the respective provider communications devices 20 of each of the healthcare providers, and/or any authorized wellness providers or fitness providers of or for the individual or patient, to each of the insurer/payer communication devices 30 of any insurers or payers of or for the individual or patient, and/or to the individual's or patients user communication/personal monitoring device(s) 40, and/or the user communication/personal monitoring device(s) 40 of or associated with any caregiver(s) of or for the individual or patient.

In a preferred embodiment, the electronic records update alert message will serve to provide the individual or patient, as well any of the individual's or patient's providers, insurers or payers, and/or any caregiver(s), with notice of the updates to individual's or patient's respective electronic healthcare record(s), electronic medical record(s), electronic dental record(s), electronic pharmacy record(s), and/or electronic behavioral healthcare record(s), and/or personal healthcare record.

Thereafter, at step 808, the central processing computer component 10A, and/or each of the respective provider communications devices 20, insurer/payer communication devices 30, and/or the individual's or patients user communication/personal monitoring device(s) 40, can store the updated electronic healthcare record(s), electronic medical record(s), electronic dental record(s), electronic pharmacy record(s), and/or electronic behavioral healthcare record(s), and/or personal healthcare record, which is stored in and by the same. In another preferred embodiment, the central processing computer component 10A, and/or each of the respective provider communications devices 20, insurer/payer communication devices 30, and/or the individual's or patients user communication/personal monitoring device(s) 40, can also simply store any new entry into the respective electronic healthcare record(s), electronic medical record(s), electronic dental record(s), electronic pharmacy record(s), and/or electronic behavioral healthcare record(s), and/or personal healthcare record, which is stored in and by the same.

At step 809, the central processing computer 10A can store any and/or all data and/or information regarding and/or contained in note, the message, the healthcare journal message, or the healthcare diary message, and/or contained in the healthcare alert message, and/or contained in the new entry made into the individual's or patient's personal healthcare record, as well as any updates and/or new entries into each of the respective electronic healthcare record(s), electronic medical record(s), electronic dental record(s), electronic pharmacy record(s), and/or electronic behavioral healthcare record(s), of and for the individual or patient, as well as any updates and/or new entries to the individual's or patient's personal healthcare record, in the database 110H and/or in the distributed ledger and Blockchain technology system component 10B of the central processing computer/distributed ledger/Blockchain technology system 10.

Thereafter, the operation of the apparatus 100 will cease at step 810.

In another preferred embodiment, each of the provider communications devices 20, the insurer/payer communication devices 30, and/or the individual's or patients user communication/personal monitoring device(s) 40, can also perform any and/or all of the herein-described steps, functions, functionalities, and/or operations, which are described as being performed by the central processing computer component 10A in the preferred embodiment of FIGS. 8A and 8B. In this regard, each of the provider communications devices 20, the insurer/payer communication devices 30, and/or the individual's or patients user communication/personal monitoring device(s) 40, can respectively process information regarding any update to any one electronic healthcare record, electronic medical record, electronic dental record, electronic pharmacy record, and/or electronic behavioral healthcare record, and/or personal healthcare record, and thereafter update all of the respective electronic healthcare record(s), electronic medical record(s), electronic dental record(s), electronic pharmacy record(s), and/or electronic behavioral healthcare record(s), and/or personal healthcare record(s), of, for, or associated with the individual or patient. In this regard, the each of the provider communications devices 20, the insurer/payer communication devices 30, and/or the individual's or patients user communication/personal monitoring device(s) 40, can perform any and/or all of the herein-described functions, functionalities, and/or operations, disclosed herein as being performed by the central processing computer component 10A.

In the above-described manner, the personal healthcare record, which is assigned to the individual or patient, can be the basis for, and can be the vehicle by which, an individual or patient can exercise control and dominion over his or her respective healthcare information, wellness information, and/or fitness information, and be continuously updated and/or alerted as to any changes in any data and/or information contained therein. In the above-described manner, the personal healthcare record, which is assigned to the individual or patient, can also be the basis for ensuring that each and every one of the respective electronic healthcare record(s), electronic medical record(s), electronic dental record(s), electronic pharmacy record(s), and/or electronic behavioral healthcare record(s), of and for the individual or patient, which are maintained and/or utilized by each respective healthcare provider, provider, or insurer or payer, of or associated with the individual or patient can be updated automatically and on a continuing basis.

In another preferred embodiment, the apparatus 100 of the present invention can also be utilized in order to allow a healthcare provider, or any other authorized healthcare, wellness, or fitness, provider, of or for an individual or patient to enter information into, and/or to change, alter, or modify, data and/or information in, the individual's or patient's personal healthcare record. In this embodiment, any and/or all of the individual's or patient's various healthcare records, electronic healthcare records, electronic medical records, electronic dental records, electronic pharmacy records, and/or electronic behavioral healthcare records, and/or personal healthcare records, can be updated with any and/or all of the herein-described data, information, messages, and/or reports, which can be entered by any healthcare provider, or by any other authorized healthcare, wellness, or fitness, provider, of or for an individual or patient, whenever the respective healthcare provider, or any other authorized healthcare, wellness, or fitness, provider, enters any data or information into their respective or various healthcare record(s), electronic healthcare record(s), electronic medical record(s), electronic dental record(s), electronic pharmacy record(s), and/or electronic behavioral healthcare record(s), of or for the individual or patient.

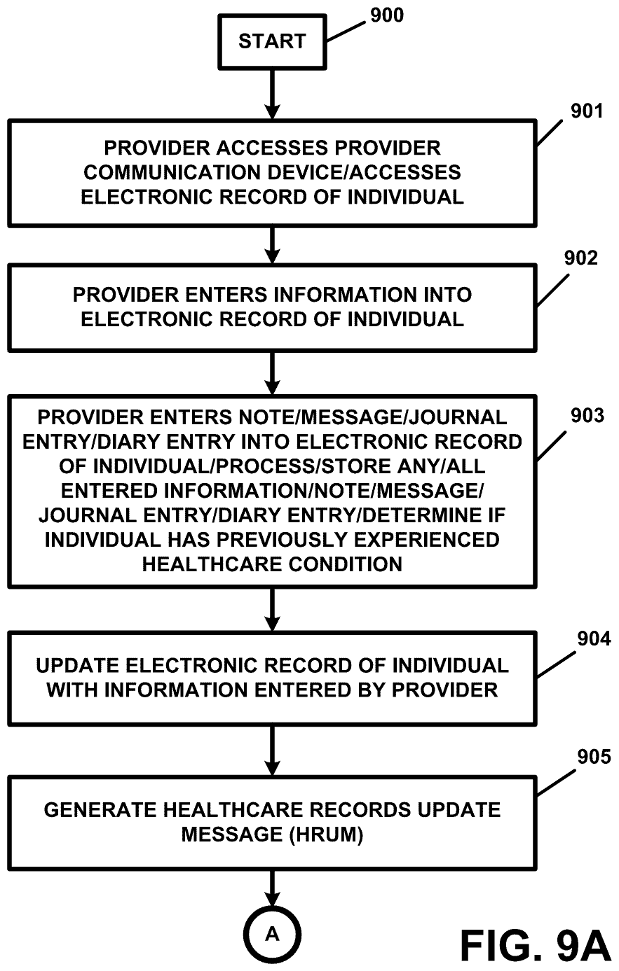

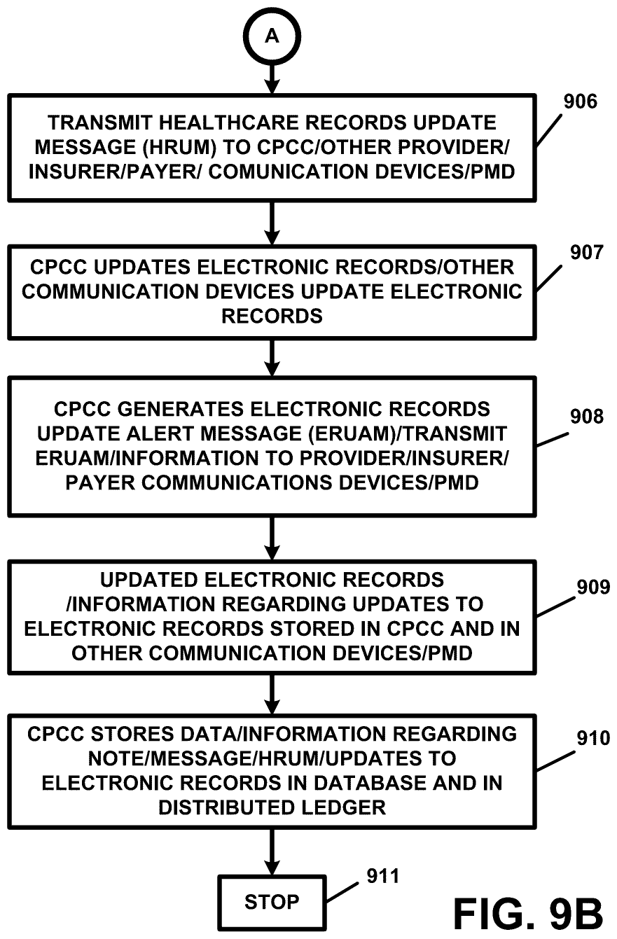

FIGS. 9A and 9B illustrate a preferred embodiment method of utilizing the apparatus 100 of the present invention, in flow diagram form. With reference to FIGS. 9A and 9B, the operation of the apparatus 100 will commence at step 900. At step 901, healthcare provider, or any other authorized healthcare, wellness, or fitness, provider (hereinafter referred to in this embodiment of FIGS. 9A and 9B as “the healthcare provider”) can access his, her, or its, provider communication device 20 and can access the respective healthcare record, electronic healthcare record, electronic medical record, electronic dental record, electronic pharmacy record, and/or electronic behavioral healthcare record, of or for the individual or patient. In a preferred embodiment, the healthcare provider can access the respective healthcare record, electronic healthcare record, electronic medical record, electronic dental record, electronic pharmacy record, and/or electronic behavioral healthcare record, of or for the individual or patient, using his, her, or its, provider communication device 20. In another preferred embodiment, the healthcare can access the respective healthcare record, electronic healthcare record, electronic medical record, electronic dental record, electronic pharmacy record, and/or electronic behavioral healthcare record, of or for the individual or patient, using any authorized provider communication device 20.

At step 902, the healthcare provider can enter information into the respective healthcare record, electronic healthcare record, electronic medical record, electronic dental record, electronic pharmacy record, and/or electronic behavioral healthcare record, of or for the individual or patient. At step 902, the healthcare provider, in a preferred embodiment, can enter information into the respective healthcare record, electronic healthcare record, electronic medical record, electronic dental record, electronic pharmacy record, and/or electronic behavioral healthcare record, of or for the individual or patient, at any time before, during, or after, an in-office visit with the individual or patient, or before, during, or after, a telehealth visit or a telehealth session with the individual or patient, or upon receiving test results, lab results, imaging results of any type or kind, treatment results, and/or diagnostic results, and/or upon receiving any information read, recorded, or obtained by, any of the herein-described user/patient monitoring systems 94, and/or any of the herein-described input devices 20D of the provider communication device 20, and/or any of the herein-described input devices 40D of the user communication/personal monitoring device 40, and/or upon any occurrence, event, or happening, which could or might dictate or suggest a need to, or a desire to, enter information into, or to change, alter, or modify, data and/or information in, the respective healthcare record, electronic healthcare record, electronic medical record, electronic dental record, electronic pharmacy record, and/or electronic behavioral healthcare record, of or for the individual or patient.

At step 903, the healthcare provider can also enter a note, message, healthcare journal entry, or healthcare diary entry, which can be stored in the provider's respective healthcare record, electronic healthcare record, electronic medical record, electronic dental record, electronic pharmacy record, and/or electronic behavioral healthcare record, of or for the individual or patient.

At step 903, the note, message, healthcare journal entry, or healthcare diary entry, entered by the healthcare provider, can include text information, an audio message or voice message, a picture or pictures, a photograph or photographs, a video recording or video recordings, and/or video and/or audio clips. In a preferred embodiment, any audio message or voice message, any picture or pictures, any photograph or photographs, any video recording or video recordings, and/or any video and/or audio clips, can be taken, obtained, or recorded, by or with the video and/or audio recording devices 20J. The note, message, healthcare journal entry, or healthcare diary entry, can be entered for any reason as determined by the healthcare provider.

In a preferred embodiment, the note, message, healthcare journal entry, or healthcare diary entry, can also be created or generated to make a note of or regarding a measurement, or a series a series of measurements, made by any one or more of the herein-described input devices 40D of the user communication/personal monitoring device 40, which can be remotely accessed and/or monitored by the healthcare provider's provider communication device 20, which measurement or series of measurements can be obtained by or from any of the herein-described or other healthcare measurement equipment or device(s) which can include, but which are not limited to, a pulse rate monitor or measurement device or equipment, a heart rate monitor of measurement device or equipment, a blood sugar monitor or measurement device or equipment, a glucose monitor or measurement device or equipment, a blood pressure monitor or measurement device or equipment, a blood alcohol monitor or measurement device or equipment, a pacemaker, a defibrillator, a thermometer for measuring an individual's body temperature, a pulse oximeter for measuring and/or monitoring oxygen saturation and/or pulse rate, an oximeter, a digital finger pulse oximeter, an electrocardiogram monitor or device, an EKG monitor or device, an electroencephalogram monitor or device, an EEG monitor or device, an electromyography monitor or device, an EMG monitor or device, a defibrillator, a pacemaker, or any other device, monitor, or measurement, device or equipment, and/or any electrical or bio-medical device or equipment, which can measure an individual's physical condition, health condition, health status, healthcare condition, or physiological condition or status, or any other biological or biometric data and/or information.

In a preferred embodiment, any pulse rate monitor or measurement device or equipment, heart rate monitor of measurement device or equipment, blood sugar monitor or measurement device or equipment, glucose monitor or measurement device or equipment, blood pressure monitor or measurement device or equipment, blood alcohol monitor or measurement device or equipment, pacemaker, defibrillator, thermometer for measuring an individual's body temperature, pulse oximeter for measuring and/or monitoring oxygen saturation and/or pulse rate, oximeter, digital finger pulse oximeter, electrocardiogram monitor or device, EKG monitor or device, electroencephalogram monitor or device, EEG monitor or device, electromyography monitor or device, EMG monitor or device, defibrillator, pacemaker, or any other device, monitor, or measurement, device or equipment, and/or any electrical or bio-medical device or equipment, which can measure an individual's physical condition, health condition, health status, healthcare condition, or physiological condition or status, or any other biological or biometric data and/or information, and/or which can be an input device 40D or 20D, and/or which can be a component of any input device 40D or 20D, can be connected via a wired connection to the user communication/personal monitoring device 40, or can be connected via a wireless connection to the user communication/personal monitoring device 40D, and/or can be connected via a Bluetooth or other suitable or appropriate wireless technology.

The user input devices 40D and 20D can also be, or can include, any one or more of, and/or any combination of, a thermometer, a digital thermometer, a stethoscope, a heart rate monitor or measurement device, a pulse rate monitor or measurement device, a blood pressure monitor or measurement device, a blood oxygen or percentage oxygen measurement device, a blood pressure measurement device, a blood analysis device or machine, a respirator, a respiration monitoring or measurement device, a dialysis machine, a dialysis device, electrocardiograph (EKG) machine or device, electrocephalograph (EEG) machine or device, electromyograph (EMG) machine or device, a magnetic resonance imaging (MRI) machine or device, X-ray machine or device, medical imaging machine or device, thermal imaging machine or device, a heart sound monitor or measurement device, a lung sound monitoring or measurement device, respiration rate monitoring or measurement device, a laparascopic device, an arthroscopic device, a vascular testing device, a catheter device, a cardiac performance testing, monitoring, or measurement device, a pulmonary performance testing, monitoring, or measurement device, a vascular system performance monitoring or measurement device, a vascular system testing, monitoring, or measurement device, a metabolism monitoring or measurement device, a sonogram imaging device, a sonogram measurement device, a sonograph device, an optical response device, an optical response measurement device, an intravenous device, an arterial blood pressure measurement or monitoring device, a respiration rate measurement or monitoring device, an ultrasound imaging device, an ultrasound measurement device, a CAT SCAN device, an optical metabolism measurement or monitoring device, a radiotelemetric device, a doppler medical device, a mammogram device, a carbon dioxide detection or measurement device, a carbon monoxide detection or measurement device, a transvascular impedance measurement or monitoring device, an ultrasonic imaging device, a bone conduction device, a brain function scan analyzer device, external pulse cardiac monitoring or measurement device, a fetal heart rate measurement, monitoring, or probing, device, an endotracheal cardiac monitoring device, a finger tip blood pressure monitoring device, a psychological monitoring device, a surgical instrument, a vital signs measurement device, ear pressure regulating device, phonocardiograph device, acoustic aneurysm detector device, blood oxygen detection device, esophageal probing device, ultrasonic probing device, ausculoscope, vital signs monitoring system, heart activity monitoring device, pulmonary activity monitoring device, sphygmomanometer, esophageal stethoscope, venous pressure measuring device, differential doppler device, physiological data measuring device, body tissue movement device, breathalyzer device, camera probing device, microscopic camera probing device, and/or any other bio-metric or physiological data measuring device(s) and/or data acquisition device data acquisition device (hereinafter referred to generally or collectively as “healthcare equipment input device”, “healthcare measurement input device”, or “healthcare monitoring input device”).

At step 903, the provider communication device 20 can process and store any and/or all information entered by the healthcare provider at step 902 into the respective healthcare record, electronic healthcare record, electronic medical record, electronic dental record, electronic pharmacy record, and/or electronic behavioral healthcare record, of or for the individual or patient. In a preferred embodiment, the note, message, healthcare journal entry, or healthcare diary entry, can also include any and/or all of the information which was entered by the healthcare provider into the respective healthcare record, electronic healthcare record, electronic medical record, electronic dental record, electronic pharmacy record, and/or electronic behavioral healthcare record, of or for the individual or patient at step 902.

At step 903, the CPU 20A and/or the provider communication device 20 can process any and/or all of the data and/or information entered into the respective healthcare record, electronic healthcare record, electronic medical record, electronic dental record, electronic pharmacy record, and/or electronic behavioral healthcare record, of or for the individual or patient, along with any information contained in the note, message, healthcare journal entry, or healthcare diary entry, and/or can process any and/or all of any data and/or information contained in any test results, lab results, imaging results of any type or kind, treatment results, and/or diagnostic results, and/or any information read, recorded, or obtained by, any of the herein-described user/patient monitoring systems 94 and/or any of the herein-described input devices 40D of the user communication/personal monitoring device 40, or the input devices 20D of the provider communication device 20, and/or any information regarding any occurrence, event, or happening.

At step 903, the CPU 20A and/or the provider communication device 20 can also process any and/or all of the herein-described data and/or information, which is entered into the respective healthcare record, electronic healthcare record, electronic medical record, electronic dental record, electronic pharmacy record, and/or electronic behavioral healthcare record, along with data and/or information already stored in the individual's or patient's personal healthcare record and/or the respective healthcare record, electronic healthcare record, electronic medical record, electronic dental record, electronic pharmacy record, and/or electronic behavioral healthcare record, of or for the individual or patient, and can determine whether or not the individual or patient has previously experienced any condition which is related or similar to any condition or state which is the subject of the healthcare provider's entry of information into the respective healthcare record, electronic healthcare record, electronic medical record, electronic dental record, electronic pharmacy record, and/or electronic behavioral healthcare record, of or for the individual or patient.

In a preferred embodiment, the CPU 20A and/or the provider communication device 20 can perform the above-described processing function, operation, or routine, using an artificial intelligence (AI) software program, software algorithm, software application, or a machine language software program, software algorithm, software application, and/or a natural language software program, software algorithm, software application. In another preferred embodiment, the CPU 20A and/or the provider communication device 20 can perform the above-described processing function, operation, or routine, without using any artificial intelligence (AI) software program, software algorithm, software application, or machine language software program, software algorithm, software application, and/or natural language software program, software algorithm, software application.

In a preferred embodiment, if the CPU 20A and/or the provider communication device 20 determines that the individual or patient has previously experienced a condition which is related or similar to the condition or state which is the subject of the healthcare provider's entry of information into the respective healthcare record, electronic healthcare record, electronic medical record, electronic dental record, electronic pharmacy record, and/or electronic behavioral healthcare record, of or for the individual or patient, the CPU 20A and/or the provider communication device 20 can generate a patient history report containing information regarding when the individual or patient experienced the condition, the date of such, the symptoms of the condition(s) previously experienced, the healthcare provider(s), if any, from which he or she received care, any diagnosis which was made, any treatment which was administered, and/or any prescriptions for medication or drugs which may have been issued to the individual or patient, and/or any other information regarding the condition(s) which was previously experienced.

In a preferred embodiment, if a patient history report is generated, then the patient history report will be included in the note, message, healthcare journal entry, or healthcare diary entry at step 903.

At step 904, the provider communication device 20 can process information regarding information which was entered by the healthcare provider into the respective healthcare record, electronic healthcare record, electronic medical record, electronic dental record, electronic pharmacy record, and/or electronic behavioral healthcare record, along with the note, message, healthcare journal entry, or healthcare diary entry, and/or any information described herein or otherwise, and can process information to effectuate, and store, an update to the respective healthcare record, electronic healthcare record, electronic medical record, electronic dental record, electronic pharmacy record, and/or electronic behavioral healthcare record, of or for the individual or patient.

At step 905, the provider communication device 20 can generate a healthcare records update message which can contain any and/or all of the information which was entered by the healthcare provider into the respective healthcare record, electronic healthcare record, electronic medical record, electronic dental record, electronic pharmacy record, and/or electronic behavioral healthcare record, along with the note, message, healthcare journal entry, or healthcare diary entry, and the patient history report and/or any information contained in the patient history report, and will effectuate, and store, an update to the respective healthcare record, electronic healthcare record, electronic medical record, electronic dental record, electronic pharmacy record, and/or electronic behavioral healthcare record, of or for the individual or patient which is stored in the provider's communication device 20.

At step 906, the provider communication 20 will transmit the healthcare records update message to the central processing computer component 10A, to any provider communication device(s) 20 of any other healthcare, wellness, or fitness, provider(s) of or for the individual or patient, to any insurer/payer communication device(s) 30 of any healthcare insurer or healthcare payer of or for the individual or patient, to the user communication/personal monitoring device 40 of or associated with the individual or patient, and/or to any user communication/personal monitoring device 40 of or associated with any caregiver of or for the individual or patient.

In a preferred embodiment, the healthcare records update message, along with any and/or all of the herein-described information contained therein, can be stored in the respective database 110H, 20H, 30H, and/or 40H, of the respective central processing computer component 10A, the respective provider communication of or associated with the healthcare provider who has entered information in the respective healthcare record, electronic healthcare record, electronic medical record, electronic dental record, electronic pharmacy record, and/or electronic behavioral healthcare record of the individual or patient, any respective provider communication device(s) 20 of any other healthcare, wellness, or fitness, provider(s) of or for the individual or patient, any respective insurer/payer communication device(s) 30 of any healthcare insurer or healthcare payer of or for the individual or patient, and/or the user communication/personal monitoring device 40 of or associated with the individual or patient, and/or any respective user communication/personal monitoring device 40 of or associated with caregiver of or for the individual or patient.

At step 907, the central processing computer component 10A will update any, each and every, and/or all of the, electronic healthcare record(s), electronic medical record(s), electronic dental record(s), electronic pharmacy record(s), and/or electronic behavioral healthcare record(s), and/or personal healthcare record(s) of, for, or associated, with each healthcare provider or other authorized provider, of or for the individual or patient. At step 907, the central processing computer component 10A will also update the personal healthcare record(s) of, for, or associated, the individual or patient.

At step 907, the central processing computer component 10A will update any, each and every, and/or all, of the herein-described electronic healthcare record(s), electronic medical record(s), electronic dental record(s), electronic pharmacy record(s), and/or electronic behavioral healthcare record(s), and/or personal healthcare record(s) of, for, or associated, with each healthcare provider or other authorized provider, and will update the personal healthcare record(s) of, for, or associated, the individual or patient, so as to include any and/or all of the data and/or information, contained in the healthcare records update message, the note, message, healthcare journal entry, or healthcare diary entry, the information which was entered by the healthcare provider into the respective healthcare record, electronic healthcare record, electronic medical record, electronic dental record, electronic pharmacy record, and/or electronic behavioral healthcare record, and/or any test results, lab results, imaging results of any type or kind, treatment results, and/or diagnostic results, and/or any information read, recorded, or obtained by, any of the herein-described user/patient monitoring systems 94, and/or any of the herein-described input devices 40D or 20D, and/or any patient history report.

At step 907, the central processing computer component 10A will update any, each and every, and/or all, of the various and respective provider's electronic healthcare record(s), electronic medical record(s), electronic dental record(s), electronic pharmacy record(s), and/or electronic behavioral healthcare record(s), and/or personal healthcare record(s) of, for, or associated with, each healthcare provider of the individual or patient, as well as update the personal healthcare record(s) of, for, or associated with, the individual or patient.

At step 907, the central processing computer component 10A will also update any, each and every, and/or all, of the various and respective provider's electronic healthcare record(s), electronic medical record(s), electronic dental record(s), electronic pharmacy record(s), and/or electronic behavioral healthcare record(s), and/or personal healthcare record(s), of, for, or associated with, each healthcare provider of the individual or patient, as well as update the personal healthcare record(s) of, for, or associated with, the individual or patient, so as to include any and/or all of the data and/or information contained in or regarding the healthcare records update message, the note, message, healthcare journal entry, or healthcare diary entry, the information which was entered by the healthcare provider into the respective healthcare record, electronic healthcare record, electronic medical record, electronic dental record, electronic pharmacy record, and/or electronic behavioral healthcare record, and/or any test results, lab results, imaging results of any type or kind, treatment results, and/or diagnostic results, and/or any information read, recorded, or obtained by, any of the herein-described user/patient monitoring systems 94, and/or any of the herein-described input devices 40D or 20D, and/or any patient history report.

In a preferred embodiment, at step 907, the central processing computer component 10A can also update any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, of or for the individual or patient, and can update the personal healthcare record of the individual or patient by using the any of the herein-described look-up tables, master index tables, and/or data and/or information conversion information, in any appropriate and suitable manner, and by using and suitable and/or appropriate algorithms and/or software, and/or processing routines, in order to perform such updates.

As and for an example, in a preferred embodiment, at step 907, the central processing computer component 10A can process any of the data and/or information contained in the healthcare records update message, the note, message, healthcare journal entry, or healthcare diary entry, the information which was entered by the healthcare provider into the respective healthcare record, electronic healthcare record, electronic medical record, electronic dental record, electronic pharmacy record, and/or electronic behavioral healthcare record, and/or any test results, lab results, imaging results of any type or kind, treatment results, and/or diagnostic results, and/or any information read, recorded, or obtained by, any of the herein-described user/patient monitoring systems 94, and/or any of the herein-described input devices 40D or 20D, and/or any patient history report, so as to identify and/or sort the respective data or information into their respective data fields and information fields and thereafter compile the data and/or information into a master template. Thereafter, using information contained in the any of the herein-described look-up tables, master index tables, and/or data and/or information conversion information, the central processing computer component 10A can then insert a new entry into any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, of or for the individual or patient, as well as into the personal healthcare record of the individual or patient.

In this manner, each and every, and/or all, of the electronic healthcare records, electronic medical records, electronic dental records, electronic pharmacy records, and/or electronic behavioral healthcare records, and/or personal healthcare records, can be interoperable with each other and with every other electronic healthcare record(s), electronic medical record(s), electronic dental record(s), electronic pharmacy record(s), and/or electronic behavioral healthcare record(s), and/or personal healthcare record(s).

In the above manner, and/or in any other appropriate and/or suitable manner, at step 907, the central processing computer component 10A can update any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, of or for the individual or patient, and can update the personal healthcare record of the individual or patient. It is important to note the central processing computer component 10A can also update any, each and every, and/or all, of the electronic healthcare record(s), electronic medical record(s), electronic dental record(s), electronic pharmacy record(s), and/or electronic behavioral healthcare record(s), and/or personal healthcare record(s), or and for any provider or individual or patient by and using any other method or methods, and/or software algorithms, software routines, and/or software programs.

At step 907, any of the herein-described provider communication devices 20, insurer/payer communication devices 30, and/or the individual's or patients user communication/personal monitoring device(s) 40, can perform the very same operations described herein as being performed by the central processing computer component 10A in order to update any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, of or for the individual or patient, and/or to update the personal healthcare record(s) of the individual or patient, with any of the herein-described data and/or information contained in or regarding the herein-described healthcare records update message, note, message, healthcare journal entry, or healthcare diary entry, information which was entered by the healthcare provider into the respective healthcare record, electronic healthcare record, electronic medical record, electronic dental record, electronic pharmacy record, and/or electronic behavioral healthcare record, and/or test results, lab results, imaging results of any type or kind, treatment results, and/or diagnostic results, and/or information read, recorded, or obtained by, any of the herein-described user/patient monitoring systems 94, and/or any of the herein-described input devices 40D or 20D, and/or any patient history report.

At step 908, the central processing computer component 10A will generate an electronic records update alert message and transmit the same, along with information contained in or regarding the update(s) to, and/or along with the updated and respective electronic healthcare record(s), electronic medical record(s), electronic dental record(s), electronic pharmacy record(s), and/or electronic behavioral healthcare record(s), and/or personal healthcare record, to each of the respective provider communications devices 20 of each of the healthcare providers, and/or any authorized wellness providers or fitness providers, of or for the individual or patient, to each of the insurer/payer communication devices 30 of any insurers or payers of or for the individual or patient, and/or to the individual's or patients user communication/personal monitoring device(s) 40, and/or the user communication/personal monitoring device(s) 40 of or associated with any caregiver(s) of or for the individual or patient.

In a preferred embodiment, the electronic records update alert message will serve to provide the individual or patient, as well any of the individual's or patient's providers, insurers or payers, and/or any caregiver(s), with notice of the updates to the individual's or patient's respective electronic healthcare record(s), electronic medical record(s), electronic dental record(s), electronic pharmacy record(s), and/or electronic behavioral healthcare record(s), and/or personal healthcare record(s).

Thereafter, at step 909, the central processing computer component 10A, and/or each of the respective provider communications devices 20, insurer/payer communication devices 30, and/or the individual's or patients user communication/personal monitoring device(s) 40, can store the updated and respective electronic healthcare record(s), electronic medical record(s), electronic dental record(s), electronic pharmacy record(s), and/or electronic behavioral healthcare record(s), and/or personal healthcare record(s), which is/are stored in and/or by the same.

In another preferred embodiment, the central processing computer component 10A, and/or each of the respective provider communications devices 20, insurer/payer communication devices 30, and/or the individual's or patients user communication/personal monitoring device(s) 40, can also store any new entry into the respective electronic healthcare record(s), electronic medical record(s), electronic dental record(s), electronic pharmacy record(s), and/or electronic behavioral healthcare record(s), ad/or personal healthcare record(s), which is/are stored in and by the same.

At step 910, the central processing computer 10A can store any and/or all data and/or information regarding and/or contained in the healthcare records update message, the note, message, healthcare journal entry, or healthcare diary entry, the information which was entered by the healthcare provider into the respective healthcare record, electronic healthcare record, electronic medical record, electronic dental record, electronic pharmacy record, and/or electronic behavioral healthcare record, and/or any test results, lab results, imaging results of any type or kind, treatment results, and/or diagnostic results, and/or any information read, recorded, or obtained by, any of the herein-described user/patient monitoring systems 94, and/or any of the herein-described input devices 40D or 20D, and/or any patient history report, and/or contained in the new entry made into the individual's or patient's personal healthcare record, as well as any updates and/or new entries into each of the respective electronic healthcare record(s), electronic medical record(s), electronic dental record(s), electronic pharmacy record(s), and/or electronic behavioral healthcare record(s), of and for the individual or patient, as well as any updates and/or new entries to the individual's or patient's personal healthcare record, in the database 110H and/or in the distributed ledger and Blockchain technology system component 10B of the central processing computer/distributed ledger/Blockchain technology system 10.

Thereafter, the operation of the apparatus 100 will cease at step 911.

In a preferred embodiment, the apparatus 100 of the present invention can be utilized in order to allow an individual or patient to self report a healthcare condition or a healthcare emergency via the user communication/personal monitoring device 40, and/or to allow the user communication/personal monitoring device 40 to automatically report a healthcare condition or a healthcare emergency regarding the individual or patient.

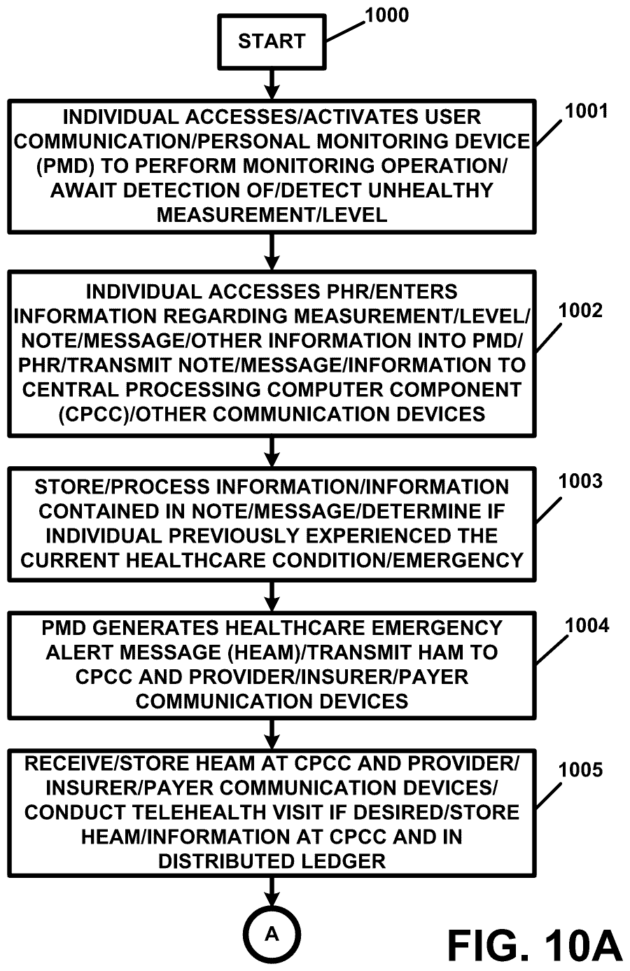

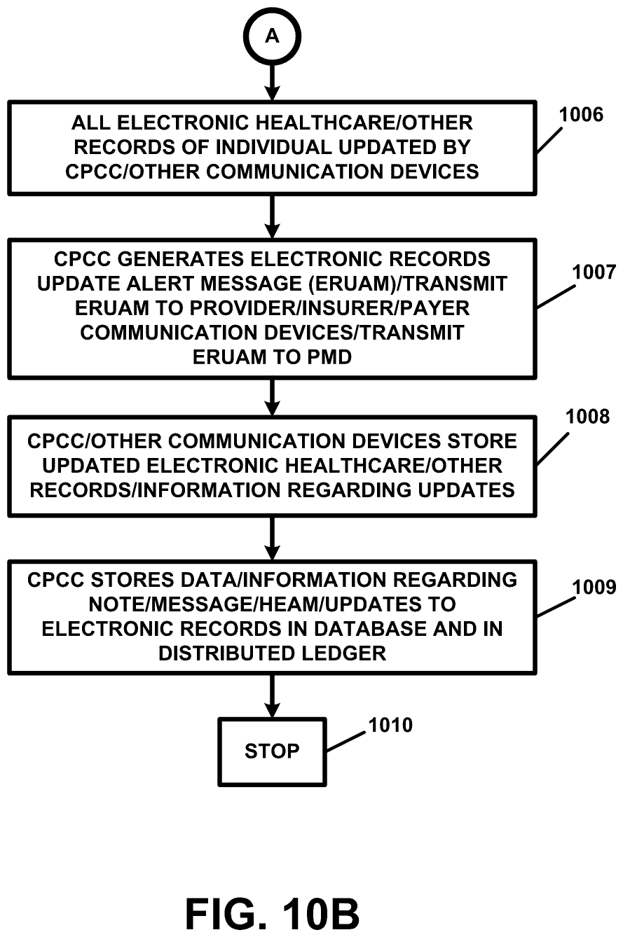

FIGS. 10A and 10B illustrate a preferred embodiment method of utilizing the apparatus 100 of the present invention, in flow diagram form. With reference to FIGS. 10A and 10B, the operation of the apparatus 100 will commence at step 1000. At step 1001, the individual or user can activate the user communication/personal monitoring device 40 to measure and/or to monitor one or more of his or her heart rate, pulse rate, blood pressure, body temperature, blood glucose level, blood sugar level, blood oxygen level, oxygen saturation level, and/or any other biometric measurement or physiological measurement which can be made by, with, or using, any of the herein-described input devices 40D, and/or any of the herein-described user/patient monitoring systems 94 (hereinafter referred to “healthcare information measurement”). In a preferred embodiment, at step 1001, the user communication/personal monitoring device 40 can continuously measure and monitor any one or more of his or her heart rate, pulse rate, blood pressure, body temperature, blood glucose level, blood sugar level, blood oxygen level, oxygen saturation level, and/or any other biometric measurement or physiological measurement which can be made by, with, or using, any of the herein-described input devices 40D, and/or any of the herein-described user/patient monitoring systems 94.

In a preferred embodiment, it is envisioned that the user communication/personal monitoring device 40 to detect and to report on any healthcare information measurement(s) which are deemed to be indicative of the individual or patient being ill, or experiencing a healthcare condition, or experiencing a healthcare emergency. For example, a very high blood pressure or a very low blood pressure for the individual or patient, a very high body temperature for the individual or patient, a very low or faint pulse rate for the individual or patient, a very low blood oxygen level for the individual or patient, a very high blood sugar level for the individual or patient, and/or any other healthcare information measurement which could be deemed dangerous for the individual or patient. In a preferred embodiment, the user communication/personal monitoring device 40 can be programmed to detect any unhealthy, abnormal, or dangerous, measurement or measurement or level or levels for or regarding any specific type or kind of healthcare information measurement.

In a preferred embodiment, if the user communication/personal monitoring device 40 detects an unhealthy, abnormal, or dangerous, measurement or measurement or level or levels for or regarding any specific type or kind of healthcare information measurement, the operation of the apparatus 100 will proceed to step 1002. In a preferred embodiment, the user communication/personal monitoring device 40 can be programmed to, and can automatically detect, an unhealthy, abnormal, or dangerous, measurement or measurement or level or levels for or regarding any specific type or kind of healthcare information measurement, and can alert the individual or patient via the display device 40E visually and/or audibly via the output device 401 which can be a speaker or an indicator light. In another preferred embodiment, the individual or patient can be provided with information regarding the unhealthy, abnormal, or dangerous, measurement or measurement or level or levels for or regarding any specific type or kind of healthcare information measurement via and by taking the measurement or measurements or by monitoring the display device 40E or the output device 1101.

At step 1002, the individual or patient can then access his or her personal healthcare record and can then enter information regarding his or her unhealthy, abnormal, or dangerous, measurement or measurement or level or levels for or regarding any specific type or kind of healthcare information measurement, and/or can enter information regarding his or her healthcare condition or any healthcare emergency, so as to enter a note, message, healthcare journal message, or healthcare diary message, which can be stored in his or her personal healthcare record. In another preferred embodiment, the user communication/personal monitoring device 40 can be programmed to automatically enter information regarding the individual's or patient's unhealthy, abnormal, or dangerous, measurement or measurement or level or levels for or regarding any specific type or kind of healthcare information measurement, for and on behalf of the individual or patient, and/or can enter information regarding the individual's or patient's healthcare condition or any healthcare emergency, so as to enter the note, message, healthcare journal message, or healthcare diary message, which can be stored in the individual's or patient's personal healthcare record, for and/or on behalf of the individual or patient.

In a preferred embodiment, the note, message, healthcare journal message, or healthcare diary message, can be transmitted, from the user communication/personal monitoring device 40 to and stored at or in the central processing computer component 10A, can be transmitted from the user communication/personal monitoring device 40 to and stored at any provider communication device(s) 20 of any healthcare, wellness, or fitness, provider of or for the individual or patient, can be transmitted from the user communication/personal monitoring device 40 to and stored at any insurer/payer communication device(s) 30 of any healthcare insurer or healthcare payer of or for the individual or patient, and/or can be transmitted from the user communication/personal monitoring device 40 to and stored at any user communication/personal monitoring device 40 of or associated with any caregiver of or for the individual or patient.

In a preferred embodiment, the note, message, healthcare journal message, or healthcare diary message, can also be stored in the respective database 110H, 20H, 30H, and/or 40H, of the respective central processing computer component 10A, provider communication device(s) 20 of any healthcare, wellness, or fitness, provider of or for the individual or patient, insurer/payer communication device(s) 30 of any healthcare insurer or healthcare payer of or for the individual or patient, and/or user communication/personal monitoring device 40 of or associated with any caregiver of or for the individual or patient.

At step 1002, the note, message, healthcare journal message, or healthcare diary message, can include information regarding any of the unhealthy, abnormal, or dangerous, measurement or measurement or level or levels for or regarding any specific type or kind of healthcare information measurement, text information, an audio message or voice message, a picture or pictures, a photograph or photographs, a video recording or video recordings, and/or video and/or audio clips, and/or any combination of the same. In a preferred embodiment, any audio message or voice message, any picture or pictures, any photograph or photographs, any video recording or video recordings, and/or any video and/or audio clips, can be taken, obtained, or recorded, by or with the video and/or audio recording devices 40J. In a preferred embodiment, the note, message, healthcare journal message, or healthcare diary message, can be created or generated to make a note of or regarding a healthcare condition or healthcare emergency experienced by or being experienced by the individual or patient.

Any data and/or information obtained, read by, or recorded by, any of the herein-described input devices 40 and healthcare equipment input devices, healthcare measurement input devices, or healthcare monitoring input devices, can also be contained or included in the note, message, healthcare journal message, or healthcare diary message, in any text, graphic, video, and/or audio, form, or combination of same.

The note, message, healthcare journal message, or healthcare diary message, can be created or generated for storage in the individual's or patient's personal healthcare record, and/or for transmission and communication to any healthcare, wellness, or fitness, providers of or for the individual or patient, for transmission and communication to any healthcare payer or healthcare insurer of or for the individual or patient, and/or for transmission and communication to any caregiver of or for the individual or patient.

At step 1003, the CPU 40A and/or the user communication/personal monitoring device 40 will store the note, message, healthcare journal message, or healthcare diary message, in the user communication/personal monitoring device 40. At step 1003, the CPU 40A and/or the user communication/personal monitoring device 40 will also store the data and/or information contained or included in the note, message, healthcare journal message, or healthcare diary message, in, and as a new entry into, the individual's or patient's personal healthcare record and in the database 40H. The new entry into the individual's or patient's personal healthcare record can include any and/or all text information, audio message or voice message, picture or pictures, photograph or photographs, video recording or video recordings, and/or video and/or audio clips, contained in the note, message, healthcare journal message, or healthcare diary message.

In a preferred embodiment, any and/or all of the herein-described electronic healthcare records, electronic medical records, electronic dental records, electronic pharmacy records, and/or electronic behavioral healthcare records, and/or personal healthcare records, can store any of the herein-described notes, messages, healthcare journal messages, or healthcare diary messages, and/or any and/or all text information, audio messages or voice messages, picture or pictures, photograph or photographs, video recording or video recordings, and/or video and/or audio clips, contained therein.

At step 1003, the CPU 40A and/or the user communication/personal monitoring device 40 can process any and/or all of the data and/or information contained in the note, message, healthcare journal message, or healthcare diary message, and/or can process any and/or all of any data and/or information obtained, read, or recorded, by any of the herein-described input devices 40 and healthcare equipment input devices, healthcare measurement input devices, or healthcare monitoring input devices, using data and/or information already stored in the individual's or patient's personal healthcare record in order to determine whether or not the individual or patient has previously experienced the healthcare condition or healthcare emergency, or a similar healthcare condition, which is currently being experienced by the individual or patient.

In a preferred embodiment, the CPU 40A and/or the user communication/personal monitoring device 40 can perform the above-described processing function, operation, or routine, using an artificial intelligence (AI) software program, software algorithm, software application, or a machine language software program, software algorithm, software application, and/or a natural language software program, software algorithm, software application. In another preferred embodiment, the CPU 40A and/or the user communication/personal monitoring device 40 can perform the above-described processing function, operation, or routine, without using any artificial intelligence (AI) software program, software algorithm, software application, or machine language software program, software algorithm, software application, and/or natural language software program, software algorithm, software application.

In a preferred embodiment, if the CPU 40A and/or the user communication/personal monitoring device 40 determines that the individual or patient has previously experienced the healthcare condition, or a similar condition, which is currently being experienced by the individual or patient, the CPU 40A and/or the user communication/personal monitoring device 40 can generate a previous condition report containing information regarding when the individual or patient experienced the condition, the date of such, the symptoms of the condition(s) previously experienced, the healthcare provider(s), if any, from which he or she received care, any diagnosis which was made, any treatment which was administered, and/or any prescriptions for medication or drugs which may have been issued to the individual or patient, and/or any other information regarding the condition(s) which was previously experienced.

In a preferred embodiment, if a previous condition report is generated, then the previous condition report will be included in the note, message, healthcare journal message, or healthcare diary message. In a preferred embodiment, the user communication/personal monitoring device 40 will provide information regarding the previous condition report, the previous condition report, and/or any information regarding the same, via the display device 40E and/or via the output device 401 which can be a speaker.

At step 1004, the CPU 40A and/or the user communication/personal monitoring device 40 can generate a healthcare emergency alert message and can transmit the same to the central processing computer component 10A, and/or to any provider communication device(s) 20 of any healthcare, wellness, or fitness, provider of or for the individual or patient, and/or to any insurer/payer communication device(s) 30 of any healthcare insurer or healthcare payer of or for the individual or patient, and/or to any user communication/personal monitoring device 40 of or associated with any caregiver of or for the individual or patient. In a preferred embodiment, the healthcare alert message can include or contain any and/or all of the information contained in the note, message, healthcare journal message, or healthcare diary message, including any text information, audio message or voice message, picture or pictures, photograph or photographs, video recording or video recordings, and/or video and/or audio clips, and/or any of the other herein-described information, as well as any information contained therein, along with a copy of any and/or all data and/or information which is contained in the new entry made into the individual's or patient's personal healthcare record.

In a preferred embodiment, the healthcare emergency alert message can include or contain any and/or all data and/or information contained in and/or regarding the new entry made into the individual's or patient's personal healthcare record. In a preferred embodiment, data and/or information contained in and/or regarding the new entry made in the individual's or patient's personal healthcare record can include any and/or all text information, audio message or voice messages, picture or pictures, photograph or photographs, video recording or video recordings, and/or video and/or audio clips, contained in the note, message, healthcare journal message, or healthcare diary message, as well as the note, message, healthcare journal message, or healthcare diary message itself, and/or any of the other herein-described information, as well as any information contained therein.

In a preferred embodiment, the healthcare emergency alert message can also contain a request for a telehealth visit or a telehealth session and/or a request for an emergency telehealth visit or an emergency telehealth session.

At step 1005, the healthcare emergency alert message can be received by, and can be stored by, the central processing computer component 10A, and/or by any provider communication device(s) 20 of any healthcare, wellness, or fitness, provider of or for the individual or patient, and/or by any insurer/payer communication device(s) 30 of any healthcare insurer or healthcare payer of or for the individual or patient, and/or by any user communication/personal monitoring device 40 of or associated with any caregiver of or for the individual or patient. At step 1005, if the healthcare emergency alert message contained a request for a telehealth visit or a telehealth session or a request for an emergency telehealth visit or an emergency telehealth session, then the a telehealth visit or telehealth session can be commenced between the user communication/personal monitoring device 40 and a provider communication device 20 or the responding healthcare provider. In a preferred embodiment, in and during the telehealth visit or telehealth session, the healthcare provider can remotely examine the individual or patient and can enter information into the respective electronic healthcare record(s), electronic medical record(s), electronic dental record(s), electronic pharmacy record(s), and/or electronic behavioral healthcare record(s), and/or personal healthcare record(s), of or for the individual or patient. In such a preferred embodiment, any data and/or information which is entered into the respective electronic healthcare record(s), electronic medical record(s), electronic dental record(s), electronic pharmacy record(s), and/or electronic behavioral healthcare record(s), and/or personal healthcare record(s), of or for the individual or patient can thereafter be processed and stored using a method which is the same, similar to, and/or analogous to, the method for utilizing the apparatus 100 in the preferred embodiment of FIGS. 9A and 9B.

At step 1005, information regarding the healthcare alert message and/or any data and/or information contained or included in the same, and/or any data and/or information relating to the same, can also be stored by the central processing computer component 10A in the database 110H and/or in the distributed ledger and Blockchain technology system component 10B of the central processing computer/distributed ledger/Blockchain technology system 10. Thereafter, the operation of the apparatus 100 will proceed to step 1006.

At step 1006, the central processing computer component 10A will update any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, of or for the individual or patient, with and so as to include any and/or all of the data and/or information, contained in and/or regarding the note, the message, the healthcare journal message, or the healthcare diary message, and/or contained in or regarding the healthcare emergency alert message, and/or contained in or regarding any new entry or new entries made into the individual's or patient's personal healthcare record.

In this regard, the central processing computer component 10A will update any, each and every, and/or all, of the various and respective provider's electronic healthcare record(s), electronic medical record(s), electronic dental record(s), electronic pharmacy record(s), and/or electronic behavioral healthcare record(s), of, for, or associated, the individual or patient, with and so as to include any and/or all of the data and/or information, contained in or regarding the note, the message, the healthcare journal message, or the healthcare diary message, and/or contained in or regarding the healthcare emergency alert message, and/or contained in or regarding the new entry made into the individual's or patient's personal healthcare record.

In a preferred embodiment, the central processing computer component 10A can update any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, of or for the individual or patient, by using the any of the herein-described look-up tables, master index tables, and/or data and/or information conversion information, in any appropriate and suitable manner, and by using and suitable and/or appropriate algorithms and/or software, and/or processing routines, in order to perform such updates.

As and for an example, in a preferred embodiment, at step 1006, the central processing computer component 10A can process any of the data and/or information contained in the note, the message, the healthcare journal message, or the healthcare diary message, and/or contained in the healthcare emergency alert message, and/or contained in the new entry made into the individual's or patient's personal healthcare record, so as to identify and/or sort the respective data or information into their respective data fields and information fields and thereafter compile the data and/or information into a master template. Thereafter, using information contained in the any of the herein-described look-up tables, master index tables, and/or data and/or information conversion information, the central processing computer component 10A can then insert a new entry into any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, of or for the individual or patient, as well as into the personal healthcare record of or for the individual or patient.

In this manner, each and every, and/or all, of the electronic healthcare records, electronic medical records, electronic dental records, electronic pharmacy records, and/or electronic behavioral healthcare records, and/or personal healthcare records, can be interoperable with each and every other electronic healthcare record(s), electronic medical record(s), electronic dental record(s), electronic pharmacy record(s), and/or electronic behavioral healthcare record(s), and/or personal healthcare record(s).

In the above manner, and/or in any other appropriate and/or suitable manner, at step 1006, the central processing computer component 10A can update any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, of or for the individual or patient. It is important to note the central processing computer component 10A can update any, each and every, and/or all, of the electronic healthcare record(s), electronic medical record(s), electronic dental record(s), electronic pharmacy record(s), and/or electronic behavioral healthcare record(s), by and using any other suitable and/or appropriate method or methods, and/or software algorithms, software routines, and/or software programs.

At step 1006, any of the herein-described provider communication devices 20, insurer/payer communication devices 30, and/or the individual's or patients user communication/personal monitoring device(s) 40, can also perform the very same operations as the central processing computer component 10A in order to update any, each and every, and/or all, of the electronic healthcare record(s), electronic medical record(s), electronic dental record(s), electronic pharmacy record(s), and/or electronic behavioral healthcare record(s), of, for, or associated, with each healthcare provider or other authorized provider, with any of the herein-described data and/or information contained in the note, the message, the healthcare journal message, or the healthcare diary message, and/or contained in the healthcare alert message, and/or contained in the new entry made into the individual's or patient's personal healthcare record.

At step 1007, the central processing computer component 10A will generate an electronic records update alert message and transmit the same, along with the update(s) to, and/or along with information contained in or regarding the update(s) to, and/or along with the updated and respective electronic healthcare record(s), electronic medical record(s), electronic dental record(s), electronic pharmacy record(s), and/or electronic behavioral healthcare record(s), and/or personal healthcare record, to each of the respective provider communications devices 20 of each of the healthcare providers, and/or any authorized wellness providers or fitness providers of or for the individual or patient, to each of the insurer/payer communication devices 30 of any insurers or payers of or for the individual or patient, and/or to the individual's or patients user communication/personal monitoring device(s) 40, and/or the user communication/personal monitoring device(s) 40 of or associated with any caregiver(s) of or for the individual or patient.

In a preferred embodiment, the electronic records update alert message will serve to provide the individual or patient, as well any of the individual's or patient's providers, insurers or payers, and/or any caregiver(s), with notice of the updates to individual's or patient's respective electronic healthcare record(s), electronic medical record(s), electronic dental record(s), electronic pharmacy record(s), and/or electronic behavioral healthcare record(s), and/or personal healthcare record.

Thereafter, at step 1008, the central processing computer component 10A, and/or each of the respective provider communications devices 20, insurer/payer communication devices 30, and/or the individual's or patients user communication/personal monitoring device(s) 40, can store the updated electronic healthcare record(s), electronic medical record(s), electronic dental record(s), electronic pharmacy record(s), and/or electronic behavioral healthcare record(s), and/or personal healthcare record, which is stored in and by the same. In another preferred embodiment, the central processing computer component 10A, and/or each of the respective provider communications devices 20, insurer/payer communication devices 30, and/or the individual's or patients user communication/personal monitoring device(s) 40, can also simply store any new entry into the respective electronic healthcare record(s), electronic medical record(s), electronic dental record(s), electronic pharmacy record(s), and/or electronic behavioral healthcare record(s), and/or personal healthcare record, which is stored in and by the same.

At step 1009, the central processing computer 10A can store any and/or all data and/or information regarding and/or contained in the note, the message, the healthcare journal message, or the healthcare diary message, and/or contained in the healthcare emergency alert message, and/or contained in the new entry made into the individual's or patient's personal healthcare record, as well as any updates and/or new entries into each of the respective electronic healthcare record(s), electronic medical record(s), electronic dental record(s), electronic pharmacy record(s), and/or electronic behavioral healthcare record(s), of and for the individual or patient, as well as any updates and/or new entries to the individual's or patient's personal healthcare record, in the database 110H and/or into the distributed ledger and Blockchain technology system component 10B of the central processing computer/distributed ledger/Blockchain technology system 10.

Thereafter, the operation of the apparatus 100 will cease at step 1010.

In any and/or all of the preferred embodiments, each of the central processing computer component(s) 10A, each of the provider communication devices 20, each of the insurer/payer communication devices 30, and each of the user communication/personal monitoring devices 40, can perform any and/or all of the interoperability functions and/or processing operations or routines for and/or involving any and/or all of the herein-described various electronic healthcare records, electronic medical records, electronic dental records, electronic pharmacy records, and/or electronic behavioral healthcare records, and/or personal healthcare records, in any appropriate manner.

In a preferred embodiment, copies of any and/or all updated and/or current and various electronic healthcare records, electronic medical records, electronic dental records, electronic pharmacy records, and/or electronic behavioral healthcare records, and/or personal healthcare records, for an individual or patient can be stored at or in each of the central processing computer component(s) 10A, each of the provider communication devices 20, each of the insurer/payer communication devices 30, and each of the user communication/personal monitoring devices 40.

In this regard, the various electronic healthcare records, electronic medical records, electronic dental records, electronic pharmacy records, and/or electronic behavioral healthcare records, and/or personal healthcare records, can be updated on any one of the central processing computer component(s) 10A, the provider communication devices 20, the insurer/payer communication devices 30, and/or the user communication/personal monitoring devices 40, and the updates electronic healthcare records, electronic medical records, electronic dental records, electronic pharmacy records, and/or electronic behavioral healthcare records, and/or personal healthcare records, can be transmitted to and stored at each of the each of the central processing computer component(s) 10A, each of the provider communication devices 20, each of the insurer/payer communication devices 30, and each of the user communication/personal monitoring devices 40.

In another preferred, copies of any and/or all updated and/or current and various electronic healthcare records, electronic medical records, electronic dental records, electronic pharmacy records, and/or electronic behavioral healthcare records, and/or personal healthcare records, for an individual or patient can be stored at or in each of the central processing computer component(s) 10A, and each of the provider communication devices 20, each of the insurer/payer communication devices 30, and each of the user communication/personal monitoring devices 40 can request access to the same, can receive and update the same, and can transmit any and/or all of the updated electronic healthcare records, electronic medical records, electronic dental records, electronic pharmacy records, and/or electronic behavioral healthcare records, and/or personal healthcare records, back to the central processing computer component(s) 10A for storage in the database 110H of the same.

In another preferred, copies of any and/or all updated and/or current and various electronic healthcare records, electronic medical records, electronic dental records, electronic pharmacy records, and/or electronic behavioral healthcare records, and/or personal healthcare records, for an individual or patient can be stored at or in each of the central processing computer component(s) 10A, and each of the provider communication devices 20, each of the insurer/payer communication devices 30, and each of the user communication/personal monitoring devices 40 can request access to the same, and can update the same via the central processing computer 10A.

In a preferred embodiment, any and/or all of the herein-described interoperability functions and/or processing operations or routines can be performed by the respective central processing computer component(s) 10A, provider communication devices 20, insurer/payer communication devices 30, and user communication/personal monitoring devices 40, automatically and/or under user control. In another preferred embodiment, any of the central processing computer component(s) 10A, the provider communication devices 20, the insurer/payer communication devices 30, and/or the user communication/personal monitoring devices 40, can perform any of the herein-described interoperability functions and/or processing operations or routines with or using artificial intelligence (AI) software programs, software algorithms, software applications, machine language software programs, software algorithms, software applications, and/or natural language software programs, software algorithms, software applications.

In any and/or all of the preferred embodiments, as well as any other embodiments, the apparatus 100 and methods of the present invention can provide, and/or provides, for the interoperability of the various electronic healthcare records, electronic medical records, electronic dental records, electronic pharmacy records, and/or electronic behavioral healthcare records, and/or personal healthcare records, of and for individuals or patients, with each other, thereby facilitating the storage and maintaining of complete and up-to-date electronic healthcare records, electronic medical records, electronic dental records, electronic pharmacy records, and/or electronic behavioral healthcare records, and/or personal healthcare records, for individuals or patients.

In any and/or all of the preferred embodiments, as well as any other embodiments, the apparatus 100 and methods of the present invention can provide, and/or provides, electronic healthcare records, electronic medical records, electronic dental records, electronic pharmacy records, and/or electronic behavioral healthcare records, and/or personal healthcare records, of and for individuals or patients, which are interoperable with other electronic healthcare records, electronic medical records, electronic dental records, electronic pharmacy records, and/or electronic behavioral healthcare records, and/or personal healthcare records, which are offered by various and different suppliers or providers of the same.

In any and/or all of the preferred embodiments, as well as any other embodiments, the apparatus 100 and methods of the present invention can provide, and/or provides, a personal healthcare record which facilitates data and/or information entry by an individual or patient and provides for the sharing of such entered data and/or information with all of the electronic healthcare records, electronic medical records, electronic dental records, electronic pharmacy records, and/or electronic behavioral healthcare records, of or used by each of, or all of, the various healthcare providers and/or healthcare insurers or payers of or for the individual or patient.

The apparatus 100 of the present invention, the methods for utilizing the same, and the various components of the apparatus, including, but not limited to, the computers and/or communications devices 10A, 20, 30, 40, and/50, described herein, serve to provide significant technological solutions in and to, and represent and evidence significant and unconventional improvements to technology in, the fields of telehealth platforms and electronic healthcare records products and/or systems.

In another preferred embodiment, as well as any and/or all of the preferred embodiments and/or any other embodiments described herein or otherwise, the apparatus 100 and methods of the present invention can also be utilized for, and in connection with or in conjunction with, pets and/or other non-human animals, and can be used with, and in connection with or in conjunction with, electronic veterinary records and/or electronic veterinary records systems. In another preferred embodiment, the user communication/personal monitoring device 40 can be adapted for use with, and in connection with or in conjunction with, any pet or any other non-human animal, and/or can adapted to be attached to, and/or worn by, the respective pet or other non-human animal, and/or any device or garment worn by the respective pet or other non-human animal.

While the present invention has been described and illustrated in various preferred and alternate embodiments, such descriptions are merely illustrative of the present invention and are not to be construed to be limitations thereof. In this regard, the present invention encompasses all modifications, variations and/or alternate embodiments, with the scope of the present invention being limited only by the claims which follow.

What is claimed is:

1. An apparatus, comprising:
a central processing computer;
a distributed ledger and blockchain technology system;
a personal monitoring device, wherein the personal monitoring device is associated with an individual, and further wherein the personal monitoring device further comprises:
a first input device;
a first processor;
a first database, wherein the first database stores a personal healthcare record of or for the individual, and further wherein the first database stores information for updating the personal healthcare record, and further wherein the first database stores information for updating one of more of an electronic healthcare record, an electronic medical record, an electronic dental record, an electronic pharmacy record, or an electronic behavioral healthcare record, of or for the individual;
a first transmitter;
a first receiver; and
a first output device; and
a provider communication device, wherein the provider communication device is associated with a healthcare provider of or for the individual, and further wherein the provider communication device further comprises:
a second input device;
a second processor;
a second database, wherein the second database stores an electronic healthcare record, an electronic medical record, an electronic dental record, an electronic pharmacy record, or an electronic behavioral healthcare record, of or for the individual, and further wherein the second database stores information for updating the electronic healthcare record, the electronic medical record, the electronic dental record, the electronic pharmacy record, or the electronic behavioral healthcare record, and for updating the personal healthcare record, and further wherein the second database stores information for updating a plurality of one of more electronic healthcare records, electronic medical records, electronic dental records, electronic pharmacy records, electronic behavioral healthcare records, or personal healthcare records, of or for the individual;
a second transmitter;
a second receiver; and
a second output device,
wherein the first input device inputs a note or a message containing healthcare information regarding a healthcare condition of the individual, wherein the note or the message contains text information, audio information, and a picture, photograph, or video information, and further wherein first database stores the note or the message, and further wherein the processor processes the healthcare information and determines whether or not the individual has previously experienced the healthcare condition, and further wherein the first processor or the personal monitoring device generates a healthcare alert message, and further wherein the first transmitter transmits the healthcare alert message to the central processing computer and to the provider communication device,
wherein the central processing computer updates the plurality of one of more of the electronic healthcare records, the electronic medical records, the electronic dental records, the electronic pharmacy records, or the electronic behavioral healthcare records, and the personal healthcare record, of or for the individual, and further wherein the central processing computer stores information regarding the note, the message, the healthcare alert message, and information regarding any updates to the plurality of any one of more of the electronic healthcare records, the electronic medical records, the electronic dental records, the electronic pharmacy records, or the electronic behavioral healthcare records, and the personal healthcare record, of or for the individual, in a database and in the distributed ledger and blockchain technology system.

2. The apparatus of claim 1, wherein the central processing computer further comprises:
a third input device;
a third processor;
a third database, wherein the third database stores any number of electronic healthcare records, electronic medical records, electronic dental records, electronic pharmacy records, electronic behavioral healthcare records, of or for the individual, and further wherein the third database stores the personal healthcare record of or for the individual, and further wherein the third database stores information for updating any or all of the electronic healthcare records, the electronic medical records, the electronic dental records, the electronic pharmacy records, or the electronic behavioral healthcare records, and for updating the personal healthcare record;
a third transmitter;
a third receiver; and
a third output device.

3. The apparatus of claim 1, wherein the first input device further comprises:
a camera;
an audio recording device; and
a video recording device.

4. The apparatus of claim 1, wherein the first output device is a display screen or a speaker.

5. The apparatus of claim 1, wherein the first processor or the personal monitoring device uses an artificial intelligence (AI) software program, algorithm, or application, a machine language software program, algorithm, or application, or a natural language software program, algorithms, or application, to determines whether or not the individual has previously experienced the healthcare condition.

6. The apparatus of claim 1, wherein the personal monitoring device updates the plurality of one of more of the electronic healthcare records, the electronic medical records, the electronic dental records, the electronic pharmacy records, or the electronic behavioral healthcare records, and the personal healthcare record, of or for the individual.

7. The apparatus of claim 1, wherein the provider communication device updates the plurality of one of more of the electronic healthcare records, the electronic medical records, the electronic dental records, the electronic pharmacy records, or the electronic behavioral healthcare records, and the personal healthcare record, of or for the individual.

8. The apparatus of claim 1, wherein the central processing computer uses information contained in a look-up table, a master index table, or data or information conversion information, to update the plurality of one of more of the electronic healthcare records, the electronic medical records, the electronic dental records, the electronic pharmacy records, or the electronic behavioral healthcare records, and the personal healthcare record, of or for the individual.

9. The apparatus of claim 1, wherein the personal monitoring device uses information contained in a look-up table, a master index table, or data or information conversion information, to update the plurality of one of more of the electronic healthcare records, the electronic medical records, the electronic dental records, the electronic pharmacy records, or the electronic behavioral healthcare records, and the personal healthcare record, of or for the individual.

10. The apparatus of claim 1, wherein the provider communication device uses information contained in a look-up table, a master index table, or data or information conversion information, to update the plurality of one of more of the electronic healthcare records, the electronic medical records, the electronic dental records, the electronic pharmacy records, or the electronic behavioral healthcare records, and the personal healthcare record, of or for the individual.

11. The apparatus of claim 1, wherein the second input device enters second healthcare information regarding the healthcare condition or regarding a second healthcare condition regarding the individual into the provider communication device, and further wherein the second healthcare information is entered into the electronic healthcare record, the electronic medical record, the electronic dental record, the electronic pharmacy record, or the electronic behavioral healthcare record, of or for the individual.

12. The apparatus of claim 11, wherein the second input device enters a second note or a second message by the healthcare provider into the provider communication device.

13. The apparatus of claim 12, wherein the second processor or the provider communication device determines whether or not the individual has previously experienced the healthcare condition or the second healthcare condition.

14. The apparatus of claim 13, wherein the second processor or the provider communication device uses an artificial intelligence (AI) software program, algorithm, or application, a machine language software program, algorithm, or application, or a natural language software program, algorithms, or application, to determine whether or not the individual has previously experienced the healthcare condition or the second healthcare condition.

15. The apparatus of claim 1, wherein first input device further comprises:
a device for measuring and for monitoring heart rate, pulse rate, blood pressure, body temperature, blood glucose level, blood sugar level, blood oxygen level, or oxygen saturation level.

16. The apparatus of claim 15, wherein the personal monitoring device is programmed to detect a second healthcare condition, wherein the second healthcare condition is a detection of an unhealthy, abnormal, or dangerous, heart rate, pulse rate, blood pressure, body temperature, blood glucose level, blood sugar level, blood oxygen level, or oxygen saturation level, and further wherein the personal monitoring device detect the unhealthy, abnormal, or dangerous, heart rate, pulse rate, blood pressure, body temperature, blood glucose level, blood sugar level, blood oxygen level, or oxygen saturation level, and further wherein the personal monitoring device provides a visual indication or an audible indication regarding the unhealthy, abnormal, or dangerous, heart rate, pulse rate, blood pressure, body temperature, blood glucose level, blood sugar level, blood oxygen level, or oxygen saturation level, via a display, via a speaker, or via an indicator light.

17. The apparatus of claim 16, wherein the personal monitoring device stores a second note or a second message regarding the second healthcare condition or the unhealthy, abnormal, or dangerous, heart rate, pulse rate, blood pressure, body temperature, blood glucose level, blood sugar level, blood oxygen level, or oxygen saturation level.

18. The apparatus of claim 17, wherein the personal monitoring device determines whether or not the individual has previously experienced the second healthcare condition or has previously experienced the unhealthy, abnormal, or dangerous, heart rate, pulse rate, blood pressure, body temperature, blood glucose level, blood sugar level, blood oxygen level, or oxygen saturation level.

19. The apparatus of claim 16, wherein the first processor or the personal monitoring device generates a healthcare emergency alert message, and further wherein the first transmitter transmits the healthcare emergency alert message to the provider communication device.

20. The apparatus of claim 19, wherein the first transmitter transmits the healthcare emergency alert message to the central processing computer.
